SEQUENCE LISTING (113 sequences)

SEQ ID NO: 1 - DNA, 23 bp, Artificial Sequence
  gRNA sequence
gagtcgcgcgctaggggccgggg

SEQ ID NO: 2 - DNA, 23 bp, Artificial Sequence
  gRNA sequence
ccggggccggggccggggcgtgg

SEQ ID NO: 3 - DNA, 23 bp, Artificial Sequence
  gRNA sequence
cccggccccggccccggccccgg

SEQ ID NO: 4 - DNA, 23 bp, Artificial Sequence
  gRNA sequence
ggacgtacctaaggcacaagtac

SEQ ID NO: 5 - DNA, 23 bp, Artificial Sequence
  gRNA sequence
ttggagataatacagcaggtggg

SEQ ID NO: 6 - DNA, 69 bp, Artificial Sequence
  HDR template
ggtgtgaaggactgcatggattccatgttcatgagtttggagataatacagcaggtggctgttgtccct

SEQ ID NO: 7 - DNA, 69 bp, Artificial Sequence
  HDR tempalte
ccacacttcctgacgtacctaaggtacaagtactcaaacctctattatgtcgtccaccgacaacaggga

SEQ ID NO: 8 - DNA, 23 bp, Artificial Sequence
  gRNA sequence
ggtatgtcagtcatagttaagac

SEQ ID NO: 9 - DNA, 23 bp, Artificial Sequence
  gRNA sequence
aaagatagtcatcttggggctgg

SEQ ID NO: 10 - DNA, 108 bp, Artificial Sequence
  HDR template
gaaggtcttcattacacctgcagctctcattttccatacattaaagatagtcatcttggggctcgtcctgccgctgcttgtcatggtcatctgctactcgggaatcct

SEQ ID NO: 11 - DNA, 75 bp, Artificial Sequence
  HDR template
cttccagaagtaatgtggacgtcgagagtaaaaggtatgtaatttctatcagtagaaccccgaccaggacgagga

SEQ ID NO: 12 - DNA, 23 bp, Artificial Sequence
  gRNA sequence
tgatcttactgattctgaaatgg

SEQ ID NO: 13 - DNA, 23 bp, Artificial Sequence
  gRNA sequence
cattatttttcattcatagttgg

SEQ ID NO: 14 - DNA, 23 bp, Artificial Sequence
  gRNA sequence
tggccgattcagaattttgttgg

SEQ ID NO: 15 - DNA, 23 bp, Artificial Sequence
  gRNA sequence
aaattggaagagtttgtttaagg

SEQ ID NO: 16 - DNA, 23 bp, Artificial Sequence
  gRNA sequence
cctgtggggcaaagtgaacgtgg

SEQ ID NO: 17 - DNA, 23 bp, Artificial Sequence
  gRNA sequence
ttgacagcagtcttctccacagg

SEQ ID NO: 18 - DNA, 23 bp, Artificial Sequence
  gRNA sequence
aacgctacacgtttcgtgtttgg

SEQ ID NO: 19 - DNA, 23 bp, Artificial Sequence
  gRNA sequence
ctgcccatccacactaggcaagg

SEQ ID NO: 20 - DNA, 23 bp, Artificial Sequence
  gRNA sequence
cagcaaccagacggacaagctgg

SEQ ID NO: 21 - DNA, 23 bp, Artificial Sequence
  gRNA sequence
cacgaagctctccgatgtgttgg

SEQ ID NO: 22 - DNA, 23 bp, Artificial Sequence
  gRNA sequenc
tatctcatacctatccctattgg

SEQ ID NO: 23 - DNA, 23 bp, Artificial Sequence
  gRNA sequence
acaactggggccaggtgcggtgg

SEQ ID NO: 24 - DNA, 23 bp, Artificial Sequence
  gRNA sequence
caatttggtcagctgggtcttgg

SEQ ID NO: 25 - DNA, 23 bp, Artificial Sequence
  gRNA sequence
tatacacagcattctctgaaagg

SEQ ID NO: 26 - DNA, 23 bp, Artificial Sequence
  gRNA sequence
ttaaacattaacggaacccccgg

SEQ ID NO: 27 - DNA, 23 bp, Artificial Sequence
  gRNA sequence
tggcgtgatctgcgcgccccagg

SEQ ID NO: 28 - DNA, 23 bp, Artificial Sequence
  gRNA sequence
ctctctgaggtaacaaattgggg

SEQ ID NO: 29 - DNA, 23 bp, Artificial Sequence
  gRNA sequence
tctcagagaggagtaaacactgg

SEQ ID NO: 30 - DNA, 23 bp, Artificial Sequence
  gRNA sequence
tcgaggagatctcgaatagaagg

SEQ ID NO: 31 - DNA, 23 bp, Artificial Sequence
  gRNA sequence
cgcctctgctctgtatcgggagg

SEQ ID NO: 32 - DNA, 23 bp, Artificial Sequence
  gRNA sequence
caacgatctgaccgccacccggg

SEQ ID NO: 33 - DNA, 23 bp, Artificial Sequence
  gRNA sequence
gccgcgcaggggccctagattgg

SEQ ID NO: 34 - DNA, 23 bp, Artificial Sequence
  gRNA sequence
cagaattgatactgactgtatgg

SEQ ID NO: 35 - DNA, 23 bp, Artificial Sequence
  gRNA sequence
aaagatagtcatcttggggctgg

SEQ ID NO: 36 - DNA, 23 bp, Artificial Sequence
  gRNA sequence
cccctatctttattgtgacgagg

SEQ ID NO: 37 - DNA, 23 bp, Artificial Sequence
  gRNA sequence
aaggaagctctattagatacagg

SEQ ID NO: 38 - DNA, 23 bp, Artificial Sequence
  gRNA sequence
cccaagacacaccatcgatctgg

SEQ ID NO: 39 - DNA, 23 bp, Artificial Sequence
  gRNA sequence
caataactttgcaacagtgaagg

SEQ ID NO: 40 - DNA, 23 bp, Artificial Sequence
  gRNA sequence
agatgccagcagatcagctcagg

SEQ ID NO: 41 - DNA, 23 bp, Artificial Sequence
  gRNA sequence
ctcagctttttctcactctatgg

SEQ ID NO: 42 - DNA, 23 bp, Artificial Sequence
  gRNA sequence
caacgtgctggtctgagtgctgg

SEQ ID NO: 43 - DNA, 23 bp, Artificial Sequence
  gRNA sequence
agctgtgggaggaagataagagg

SEQ ID NO: 44 - DNA, 23 bp, Artificial Sequence
  gRNA sequence
agcctccaccagctcctcctcgg

SEQ ID NO: 45 - DNA, 23 bp, Artificial Sequence
  gRNA sequence
atgcaggcatcctcagctacagg

SEQ ID NO: 46 - DNA, 23 bp, Artificial Sequence
  gRNA sequence
gctaggccacgccgaggtcctgg

SEQ ID NO: 47 - DNA, 23 bp, Artificial Sequence
  gRNA sequence
agttatggcgacgaaggccgtgg

SEQ ID NO: 48 - DNA, 23 bp, Artificial Sequence
  gRNA sequence
catgaacacggaatccatgcagg

SEQ ID NO: 49 - DNA, 23 bp, Artificial Sequence
  gRNA sequence
ttggagataatacagcaggtggg

SEQ ID NO: 50 - DNA, 23 bp, Artificial Sequence
  gRNA sequence
gagtcgcgcgctaggggccgggg

SEQ ID NO: 51 - DNA, 23 bp, Artificial Sequence
  gRNA sequence
ccggggccggggccggggcgtgg

SEQ ID NO: 52 - DNA, 23 bp, Artificial Sequence
  gRNA sequence
cccggccccggccccggccccgg

SEQ ID NO: 53 - DNA, 23 bp, Artificial Sequence
  gRNA sequence
tgccgcacgccccctggcagcgg

SEQ ID NO: 54 - DNA, 23 bp, Artificial Sequence
  gRNA sequence
ggcggcggaggcggcggcggcgg

SEQ ID NO: 55 - DNA, 23 bp, Artificial Sequence
  gRNA sequence
ccaggcccaggcccaggcccagg

SEQ ID NO: 56 - DNA, 23 bp, Artificial Sequence
  gRNA sequence
cctgcgcctgccctgggaacggg

SEQ ID NO: 57 - DNA, 23 bp, Artificial Sequence
  gRNA sequence
cctgggcctgggcctgggcctgg

SEQ ID NO: 58 - DNA, 23 bp, Artificial Sequence
  gRNA sequence
ccatgtgtactacatccacacgg

SEQ ID NO: 59 - DNA, 23 bp, Artificial Sequence
  gRNA sequence
aaactcactggagtttgacgtgg

SEQ ID NO: 60 - DNA, 23 bp, Artificial Sequence
  gRNA sequence
agcggtaactaagattagtatgg

SEQ ID NO: 61 - DNA, 23 bp, Artificial Sequence
  gRNA sequence
ttccaactgttcatcggctgagg

SEQ ID NO: 62 - DNA, 23 bp, Artificial Sequence
  gRNA sequence
gtccgtgctgtccgcctcggagg

SEQ ID NO: 63 - DNA, 23 bp, Artificial Sequence
  gRNA sequence
tggggccgcagggcgtggatggg

SEQ ID NO: 64 - DNA, 23 bp, Artificial Sequence
  gRNA sequence
cataaattttacatttgagttgg

SEQ ID NO: 65 - DNA, 23 bp, Artificial Sequence
  gRNA sequence
gatgatataacaaatcaatatgg

SEQ ID NO: 66 - DNA, 23 bp, Artificial Sequence
  gRNA sequence
gagattgattttctaataaacgg

SEQ ID NO: 67 - DNA, 23 bp, Artificial Sequence
  gRNA sequence
ccgccgatatattacagaacagg

SEQ ID NO: 68 - DNA, 23 bp, Artificial Sequence
  gRNA sequence
accccgcctgtaacacgagcagg

SEQ ID NO: 69 - DNA, 23 bp, Artificial Sequence
  gRNA sequence
taccacagagtctagactcgtgg

SEQ ID NO: 70 - DNA, 23 bp, Artificial Sequence
  gRNA sequence
gtttatgttgaatagcattgagg

SEQ ID NO: 71 - DNA, 23 bp, Artificial Sequence
  gRNA sequence
aaccgagtccgatgaaaaaaagg

SEQ ID NO: 72 - DNA, 23 bp, Artificial Sequence
  gRNA sequence
gcttcgggcgcttcttgcagagg

SEQ ID NO: 73 - DNA, 23 bp, Artificial Sequence
  gRNA sequence
ctgggggcagccgatacccgggg

SEQ ID NO: 74 - DNA, 23 bp, Artificial Sequence
  gRNA sequence
ccaggatccaactgcctatgtgg

SEQ ID NO: 75 - DNA, 23 bp, Artificial Sequence
  gRNA sequence
tgtggccatgtggagtgacgtgg

SEQ ID NO: 76 - DNA, 23 bp, Artificial Sequence
  gRNA sequence
catctaattcaacaagaattggg

SEQ ID NO: 77 - DNA, 23 bp, Artificial Sequence
  gRNA sequence
gggcaaaaattctctgtcagtgg

SEQ ID NO: 78 - DNA, 23 bp, Artificial Sequence
  gRNA sequence
ccctcctattaccgaggctgtgg

SEQ ID NO: 79 - DNA, 23 bp, Artificial Sequence
  gRNA sequence
ttgatactcctgggacaaatggg

SEQ ID NO: 80 - DNA, 23 bp, Artificial Sequence
  gRNA sequence
ggtatgattagaatcaatggcga

SEQ ID NO: 81 - DNA, 23 bp, Artificial Sequence
  gRNA sequence
ttccaactgttcatcggctgagg

SEQ ID NO: 82 - DNA, 84 bp, Artificial Sequence
  gRNA sequence
atatcttcctaattactatgcttatcttggttaccgctaacaacctattccaactgttcatcggatgggagggcgtaggatcat

SEQ ID NO: 83 - DNA, 84 bp, Artificial Sequence
  HDR template
tatagaaggattaatgatacgaatagaaccaatggcgattgttggataaggttgacaagtagcctaccctcccgcatcctagta

SEQ ID NO: 84 - DNA, 23 bp, Artificial Sequence
  gRNA sequence
ggtcctggagccgcaccggatcg

SEQ ID NO: 85 - DNA, 23 bp, Artificial Sequence
  gRNA sequence
agttatggcgacgaaggccgtgg

SEQ ID NO: 86 - DNA, 23 bp, Artificial Sequence
  gRNA sequence
ggaggctccgcctgtcgtgcctg

SEQ ID NO: 87 - DNA, 23 bp, Artificial Sequence
  gRNA sequence
tggggccgcagggcgtggatggg

SEQ ID NO: 88 - DNA, 23 bp, Artificial Sequence
  gRNA sequence
ggttgagtttacattttaaatac

SEQ ID NO: 89 - DNA, 23 bp, Artificial Sequence
  gRNA sequence
gatgatataacaaatcaatatgg

SEQ ID NO: 90 - DNA, 23 bp, Artificial Sequence
  gRNA sequence
ggcaaataatcttttagttagag

SEQ ID NO: 91 - DNA, 23 bp, Artificial Sequence
  gRNA sequence
ccgccgatatattacagaacagg

SEQ ID NO: 92 - DNA, 23 bp, Artificial Sequence
  gRNA sequence
ggacgagcacaatgtccgcccca

SEQ ID NO: 93 - DNA, 23 bp, Artificial Sequence
  gRNA sequence
taccacagagtctagactcgtgg

SEQ ID NO: 94 - DNA, 23 bp, Artificial Sequence
  gRNA sequence
ggagttacgataagttgtatttg

SEQ ID NO: 95 - DNA, 23 bp, Artificial Sequence
  gRNA sequence
aaccgagtccgatgaaaaaaagg

SEQ ID NO: 96 - DNA, 23 bp, Artificial Sequence
  gRNA sequence
ggagacgttcttcgcgggcttcg

SEQ ID NO: 97 - DNA, 23 bp, Artificial Sequence
  gRNA sequence
ctgggggcagccgatacccgggg

SEQ ID NO: 98 - DNA, 23 bp, Artificial Sequence
  gRNA sequence
gggttaagaacaacttaatctac

SEQ ID NO: 99 - DNA, 23 bp, Artificial Sequence
  gRNA sequence
gggcaaaaattctctgtcagtgg

SEQ ID NO: 100 - DNA, 23 bp, Artificial Sequence
  gRNA sequence
ggtgtcggagccattatcctccc

SEQ ID NO: 101 - DNA, 23 bp, Artificial Sequence
  gRNA sequence
ttgatactcctgggacaaatggg

SEQ ID NO: 102 - DNA, 23 bp, Artificial Sequence
  gRNA sequence
ggtctagctaccacacagaaccc

SEQ ID NO: 103 - DNA, 23 bp, Artificial Sequence
  gRNA sequence
caataactttgcaacagtgaagg

SEQ ID NO: 104 - DNA, 39 bp, Artificial Sequence
  Primer
gggtctagcaagagcaggtgtgggtttaggaggtgtgtg

SEQ ID NO: 105 - DNA, 39 bp, Artificial Sequence
  Primer
gccccgaccacgccccggccccggccccggcccctagcg

SEQ ID NO: 106 - DNA, 7669 bp, Artificial Sequence
  Plasmid sequence
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtgtgcaactcctaatctcaggtgatctacccaccttggcctcccaaattgctgggattacaggcgtgaaccactgctcccttccctgtccttcctgcaggaagcttccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacaagcttcctaggtatcccggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgactcactgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccggctggggaggggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttggtacctttaagaccaatgacttacaaggcagctgtagatcttagccactttttaaaagaaaaggggggacaccggtaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagacaccttggatccgagtcgcgcgctaggggccggttttagagctagaaatagcaagttaaaataaggctagtccgttatcaacttgaaaaagtggcaccgagtcggtgctttttttgaattcgagggcctatttcccatgattccttcatatttgcatatacgatacaaggctgttagagagataattggaattaatttgactgtaaacacaaagatattagtacaaaatacgtgacgtagaaagtaataatttcttgtgtagtttgcagttttaaaattatgttttaaaatggactatcatatgcttaccgtaacttgaaagtatttcgatttcttggctttatatatcttgtggaaaggacgaaacaccgccggggccggggccggggcggttttagagctagaaatagcaagttaaaataaggctagtccgttatcaacttgaaaaagtggcaccgagtcggtgctttttttgaattcgaacgctgacgtcatcaacccgctccaaggaatcgcgggcccagtgtcactaggcgggaacacccagcgcacgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagacaccttggatcccccggccccggccccggcccgttttagagctagaaatagcaagttaaaataaggctagtccgttatcaacttgaaaaagtggcaccgagtcggtgctttttttgaattcgtagtagttcatgtcatcttattattcagtatttataacttgcaaagaaatgaatatcagagagtgagaggggcgcgcc

SEQ ID NO: 107 - DNA, 7345 bp, Artificial Sequence
  Plasmid sequence
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtgtgcaactcctaatctcaggtgatctacccaccttggcctcccaaattgctgggattacaggcgtgaaccactgctcccttccctgtccttcctgcaggaagcttccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacaagcttcctaggtatcccggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgactcactgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccggctggggaggggctagcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttactagttttaagaccaatgacttacaaggcagctgtagatcttagccactttttaaaagaaaaggggggacaccggtaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagacaccttggatccggcattcgtggatggcgatcgttttagagctagaaatagcaagttaaaataaggctagtccgttatcaacttgaaaaagtggcaccgagtcggtgctttttttgaattcgagggcctatttcccatgattccttcatatttgcatatacgatacaaggctgttagagagataattggaattaatttgactgtaaacacaaagatattagtacaaaatacgtgacgtagaaagtaataatttcttgtgtagtttgcagttttaaaattatgttttaaaatggactatcatatgcttaccgtaacttgaaagtatttcgatttcttggctttatatatcttgtggaaaggacgaaacaccgaccaattcagggaccagcgcgttttagagctagaaatagcaagttaaaataaggctagtccgttatcaacttgaaaaagtggcaccgagtcggtgctttttttgaattcgtagtagttcatgtcatcttattattcagtatttataacttgcaaagaaatgaatatctcagagagtgagagggcggccgc

SEQ ID NO: 108 - DNA, 6747 bp, Artificial Sequence
  Plasmid sequence
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtgtgcaactcctaatctcaggtgatctacccaccttggcctcccaaattgctgggattacaggcgtgaaccactgctcccttccctgtccttcctgcaggaagcttccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacaagcttcctaggtatcccggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgactcactgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccggctggggagggacgcgtgccaccatgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtcgaccccggtgcagccaatggccctgattgtgctggggggcgtcgccggcctcctgcttttcattgggctaggcatcttcttctgtgtcaggtgccggcactccggactcagatctaaatgaactagtgaagttcctattctctagaaagtataggaacttcacttcattttccgtcttcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaccccaaaggtgaccgtccgttttagagctagaaatagcaagttaaaataaggctagtccgttatcaacttgaaaaagtggcaccgagtcggtgcttttttgctccgcggcacgagaactcaaagccccggggcctgggtcccacgcggggtcccttacccagggtgccccgggcgctcatttgcatgtcccacccaacaggtaaacctgacaggtcatcgcggccaggtacgacctggcggtcagagcaccaaacatacgagccttgtgatgagttccgttgcatgaaattctcccaaaggctccaagatggacaggaaagggcgcggttcggtcaccgtaagtagaataggtgaaagactcccgtgccttataaggcctgtgggtgacttcttctcaccgaagtgtgatcacttgggtgggttttagagctagaaatagcaagttaaaataaggctagtccgttatcaacttgaaaaagtggcaccgagtcggtgcttttttgaagacacaccgctctcaatattggccattagccatattattcattggttatatagcataaatcaatattggctattggccattgcatacgttgtatctatatcataatatgtacatttatattggctcatgtccaatatgaccgccatgttggcattgattattggtcgactttaagaccaatgacttacaaggcagctgtagatcttagccactttttaaaagaaaaggggggacaccggtaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagacaccttggatccagatgactatctttaatgtcgttttagagctagaaatagcaagttaaaataaggctagtccgttatcaacttgaaaaagtggcaccgagtcggtgctttttttgaattcgagggcctatttcccatgattccttcatatttgcatatacgatacaaggctgttagagagataattggaattaatttgactgtaaacacaaagatattagtacaaaatacgtgacgtagaaagtaataatttcttgtgtagtttgcagttttaaaattatgttttaaaatggactatcatatgcttaccgtaacttgaaagtatttcgatttcttggctttatatatcttgtggaaaggacgaaacaccgggtcctgccgctgcttgtcagttttagagctagaaatagcaagttaaaataaggctagtccgttatcaacttgaaaaagtggcaccgagtcggtgctttttttgaattcgtagtagttcatgtcatcttattattcagtatttataacttgcaaagaaatgaatatctcagagagtgagagggcggccgc

SEQ ID NO: 109 - RNA, 4900 nt, Artificial Sequence
  Cas9n mRNA
auggcucccaagaagaagcgaaaggugggcauccacggcgugcccgcugccgacaaaaaguauaguaucggacuggcuauuggcacuaacagcgugggaugggccgucaucaccgacgaguacaaagugccaagcaagaaguucaagguccugggaaacaccgauagacacaguaucaagaaaaaucugauuggagcccugcuguucgacucaggggagacagcugaagcaacuaggcugaaaagaacagcuaggagacgguauacucgccgaaagaaucggaucugcuaccuccaggagauuuucuccaacgaaauggccaagguggacgauaguuucuuucaucgccuggaggaaucauuccuggucgaggaagauaagaaacacgagaggcaucccaucuuuggcaacauuguggacgaggucgcuuaucacgaaaaguacccuacaaucuaucaucugcggaagaaacugguggacagcacugauaaggcagaccugcgccugaucuaucuggcccuggcucacaugauuaaguucagggggcauuuucugaucgagggcgaucugaacccagacaauuccgauguggacaagcuguucauccagcugguccagacauacaaucagcuguuugaggaaaaccccauuaaugcaucugggguggacgcaaaagccauccugagugccagacugucuaagaguaggagacuggagaaccugaucgcucagcugccaggcgaaaagaaaaacggccuguuuggaaaucugauugcacugucacugggacugacccccaacuucaagagcaauuuugaucuggccgaggacgcuaagcuccagcugagcaaggacaccuacgacgaugaccuggauaaccugcuggcucagaucggcgaucaguacgcagaccuguuccuggccgcuaagaaucugucugacgccauccugcugagugauauucugagagugaacaccgagauuacaaaagccccccugucagcuagcaugaucaagagauaugacgagcaccaucaggaucugacccugcugaaggcucuggugcggcagcagcugccugagaaguacaaagaaaucuucuuugaucagagcaagaauggguacgccggcuauauugacggcggagcuucccaggaggaguucuacaaguuuaucaaaccuauucuggagaagauggacggcacugaggaacugcuggugaaacugaaucgggaagaccugcugcggaagcagcgcaccuucgauaacggcagcaucccucaccagauucaucugggagagcugcacgcaauccugcggcgccaggaagacuucuacccauuucugaaggauaaccgggagaagaucgaaaaaauucugacuuuccgcauccccuacuauguggggccucuggcaagaggcaauucccgguuugccuggaugacccgcaagucugaggaaacaaucacucccuggaacuucgaggaaguggucgauaagggcgcuuccgcacagucuuucauugagaggaugacaaauuuugacaagaaccugccaaaugaaaaagugcugcccaagcacagccugcuguacgaguauuucaccgucuauaacgaacugacaaaggugaaauacgucacugagggcaugagaaagccugccuuccuguccggagaacagaagaaagcuaucguggaccugcuguuuaaaaccaaucggaaggugacagucaagcagcugaaagaggacuacuucaagaaaauugaauguuucgauucuguggagaucaguggggucgaagacagguuuaacgccucucugggcaccuaccacgaucugcugaagaucauuaaggauaaagacuuccuggacaacgaggaaaaugaggacauccuggaggacauugugcugacccugacacuguuugaggaucgggaaaugaucgaggaacgccugaagaccuacgcccaucuguucgaugacaaagugaugaaacagcugaagcgaaggagauacacuggguggggccgacugagcaggaagcugaucaauggcauucgcgacaaacagaguggaaagacaauccuggacuuucugaagucagauggcuucgcuaacaggaauuuuaugcagcugauucacgaugacucucugacuuucaaagaggacauccagaaggcacagguguccggacagggggacucucugcacgagcauaucgcaaaccuggccgggagcccugccaucaagaaaggcauccuccagaccgugaaggugguggacgagcuggugaaagucaugggaagacauaagccagaaaacaucgugauugagauggccagggagaaucagaccacacagaaagggcagaagaacucucgggagcgcaugaaacgcaucgaggaaggaauuaaggaacuggggagucagauccugaaagagcaccccguggaaaacacacagcuccagaaugagaagcuguaucuguacuaccuccagaauggccgcgauauguacguggaccaggagcuggauauuaaccgacugucagauuaugacguggaucauaucgucccacagucauuccugaaagaugacagcauugacaauaaggugcugacccgcagcgacaaaaaccgaggaaagagugauaauguccccucagaggaaguggucaagaaaaugaagaacuacuggaggcagcugcugaaugccaaacugaucacccagcgaaaguuugauaaccugacaaaagcugagagggggggccuguccgaacuggacaaagcaggcuucaucaagcgacagcugguggagacaaggcagaucacaaagcacgucgcucagauccuggacagcaggaugaacaccaaguacgaugagaaugacaaacugauccgggaagugaaggucauuacacugaagucaaaacuggugagcgacuuuaggaaagauuuccaguucuacaaggucagagagaucaacaacuaccaccaugcucaugacgcauaccugaacgcaguggucgggacugcccugauuaagaaauacccuaaacuggagucugaguucguguacggcgacuauaagguguacgaugucagaaaaaugaucgccaagagcgagcaggaaauuggcaaagccaccgcuaaguauuucuuuuacuccaacaucaugaauuucuuuaagacugagaucacccuggcaaauggcgaaauccgaaagaggccacugauugagacuaacggagagacaggggaaaucgugugggacaaaggaagagauuuugcuaccgugcggaagguccugaguaugccccaagugaauauugucaagaaaacagaggugcagacuggaggguucaguaaggaaucaauucugccuaaacgcaacagcgauaagcugaucgcccgaaagaaagacugggaccccaagaaguauggcggauucgacuccccaaccguggcuuacucuguccugguggucgcaaagguggagaagggaaaaagcaagaaacugaaauccgucaaggaacugcuggggaucacaauuauggagaggagcagcuucgaaaagaauccuaucgauuuucuggaggccaaaggguauaaggaagugaagaaagaccugaucaucaagcugccaaaguacucucuguuugagcuggaaaacggcagaaagcggaugcuggcaagugccggcgagcugcaaaaaggaaaugaacuggcccugcccucaaaguacgugaacuuccuguaucuggcuagccacuacgagaagcugaaaggcuccccugaggauaacgaacagaaacagcuguuuguggagcagcacaagcauuaucuggacgagaucauugaacagauuagcgaguucuccaaacgcgugauccuggcugacgcaaaucuggauaagguccugucugcauacaacaaacacagggacaagccaaucagagagcaggccgaaaauaucauucaucuguucacucugaccaaccugggagcccccgcagccuucaaguauuuugacacuaccaucgaucgcaaacgauacacaagcacuaaggaggugcuggaugcuacccugauccaccagagcauuacugggcuguacgagacaaggaucgaccugucccagcuggggggagacaaacgcccagccgccaccaagaaagcaggacaggcaaagaagaagaaguaggcggccgcgaauaugaucagguaacuuucgaccgacuuuccccaagagaaaauuccuagaaauugaacaaaaauguuuccacuggcuuuugccuguaagaaaaaaaauguacccgagcacauagagcuuuuuaauagcacuaaccaaugccuuuuuagauguauuuuugauguauauaucuauuauucaaaaaaucauguuuauuuugaguccuaggacuuaaaauuagucuuuuguaauaucaagcaggacccuaagaugaagcugagcuuuugaugccaggugcaaucuacuggaaauguagcacuuacguaaaacauuuguuucccccacaguuuuaauaagaacagaucaggaauucuaaauaaauuucccaguuaaagauuauugugacuucacuguauauaaacauauuuuuauacuuuauugaaaggggacaccucuauauucuuccaucaucacuguaaagacaaauaaaugauuauauucacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 110 - DNA, 20 bp, Artificial Sequence
  primer
atactgtggccacaagcatg

SEQ ID NO: 111 - DNA, 20 bp, Artificial Sequence
  primer
gttccccatcctaaatgtgg

SEQ ID NO: 112 - DNA, 20 bp, Artificial Sequence
  gRNA sequence
ggcattcgtggatggcgatc

SEQ ID NO: 113 - DNA, 20 bp, Artificial Sequence
  gRNA sequence
accaattcagggaccagcgc

(19) United States
(12) Patent Application Publication
(10) Pub. No.: US 2019/0330603 A1
(43) Pub. Date: Oct. 31, 2019

(54) CRISPR-CAS SYSTEM, MATERIALS AND METHODS

(71) Applicant: GENESIS TECHNOLOGIES LIMITED, Warrens, St. Michael (BB)

(72) Inventors: Jan-Eric AHLFORS, Laval (CA); Sarathi MANI, Laval (CA)

(21) Appl. No.: 16/310,515

(22) PCT Filed: Jun. 16, 2017
(86) PCT No.: PCT/IB2017/053599

§ 371 Date: Dec. 17, 2018

Related U.S. Application Data
(60) Provisional application No. 62/351,398, filed on Jun. 17, 2016.

(51) Int. Cl. C12N 9/22; C12N 15/11; C12N 15/85; C12N 15/10; A61P 35/00; A61P 35/02

(52) U.S. Cl. CPC C12N 9/22; C12N 15/11; C12N 15/85; C12N 2310/20; A61P 35/00; A61P 35/02; C12N 15/102

(57) ABSTRACT

Methods and systems for targeted genomic modification within a target genome region (TGR) in a mammalian cell. In particular, there is provided a CRISPR/Cas9 system comprising: one or more guide RNA (gRNA) comprising a CRISPR RNA (crRNA) and a trans-activating crRNA (tracrRNA) linked together, the gRNA binding with sequence specificity to a target DNA sequence in the TGR that is adjacent to a PAM sequence; a Cas9n protein; and an optional repair template for homology-directed repair (HDR). The mammalian cell may be contacted with the CRISPR/Cas9 system such that the TGR is modified, forming a modified-TGR, the one or more gRNA and/or the optional repair template selected such that the modified-TGR cannot be further modified by the CRISPR/Cas9 system. A third gRNA may be selected such that the CRISPR/Cas9 system can only bind and/or modify the third target DNA sequence if the TGR comprises a disease-causing modification.

### CROSS-REFERENCE TO RELATED APPLICATIONS

The present application claims priority to U.S. Provisional Patent Application No. 62/351,398 filed on Jun. 17, 2016, the entirety of which is incorporated herein by reference.

### FIELD

The present disclosure relates to compositions, methods and systems for targeted genomic modification and targeted regulation of gene expression in mammalian cells, including in human cells. In particular, type II CRISPR-Cas systems of Cas9 enzymes, guide RNAs and associated specific PAMs are described.

### BACKGROUND

The clustered regularly interspaced short palindromic repeats/CRISPR associated system (CRISPR/Cas) is a microbial adaptive immune system that evolved within the bacterial and archeal organisms as a defense against invading genetic materials such as viruses and plasmids. The CRISPR system has enormous potential for adaptation for genome editing in humans, animals and other organisms.

The CRISPR system uses RNA-guided nucleases to cleave foreign genetic elements. The CRISPR/Cas system is generally classified into three major divisions known as Type-I, Type-II and Type-III as well as several subdivisions based on the Cas genes (Chylinski, K. et al., Nucleic Acids Research 42(10):6091-105, 2014). The CRISPR nuclease system only requires 3 components, which include the Cas9 protein (a nuclease), CRISPR RNA (crRNA) and trans-activating crRNA (tracrRNA) for genome editing (see FIG. 1A). The crRNA and the tracrRNA can be attached together in a guide RNA (gRNA) (see FIG. 1B). As initially demonstrated in the original type II system of Streptococcus pyogenes, tracrRNA binds to the invariable repeats of precursor CRISPR RNA (pre-crRNA) forming a dual-RNA that is essential for both RNA co-maturation by RNase III in the presence of Cas9 and invading DNA cleavage by Cas9. Cas9 is guided by the duplex formed between mature activating tracrRNA and targeting crRNA and introduces site-specific double-stranded DNA (dsDNA) breaks in the invading cognate DNA.

DNA cleavage specificity is determined by two parameters: the variable, spacer-derived sequence of crRNA targeting the protospacer sequence (a protospacer is defined as the sequence on the DNA target that is complementary to the spacer of crRNA) and a short sequence, the Protospacer Adjacent Motif (PAM), located immediately downstream of the protospacer on the non-target DNA strand. It is known that a gene-specific 20-nucleotide guide sequence inserted in the gRNA can be sufficient to direct the Cas9 protein to that specific gene's target sequence in the genome to execute the gene-editing process (FIG. 1B). The 20-nucleotide guide sequence together with the PAM sequence is essential for Cas9 protein to recognize the correct gene target location in the host's genome.

Further, successful binding of Cas9 protein to a target DNA sequence requires a sequence that is complementary to the gRNA sequence and must be immediately followed by a Protospacer Adjacent Motif (PAM) sequence at the 3′ end of the 20 bp target sequence (see FIG. 2A). The 20-nucleotide guide sequence together with the PAM sequence is essential for Cas9 protein to recognize the correct gene target location in the host's genome. However, the PAM sequence is not required in the gRNA sequence. The absence of the PAM sequence in the target DNA sequence (also referred to as a target genomic DNA or gDNA, when the target DNA sequence is present in the genome of a cell) can prevent the binding of Cas9 protein to the target DNA sequence (see FIG. 2B). By delivering the Cas9 plasmid or protein with appropriate gRNAs into a cell, Cas9 can be utilized to cleave the target DNA (or gDNA) towards almost any desired location (FIG. 3A). The PAM sequence for each Cas9 protein varies based on the bacterial species from which the Cas9 protein is derived. For example, the Cas9 from Streptococcus pyogenes (SpCas9) requires a 5′-NGG PAM sequence.

With the guidance of gRNA, Cas9 protein promotes genome editing at a pre-defined target sequence by inducing a double stranded break (DSB) in the DNA. Following the DSB induced by Cas9 protein, the target sequence can be repaired by the cellular repair machinery via either non-homologous end-joining (NHEJ) or homology-directed repair (HDR). In the absence of a donor template, NHEJ activates to re-ligate the DSBs, resulting in a genetic scar in the form of insertion/deletion (indel) mutations at the target sequence. This resulting NHEJ scar within the target sequence can cause gene knockouts, as the indels occurring within a coding exon can lead to frameshift mutations and premature stop codons.

In the presence of a donor template, HDR is an alternative major DNA repair pathway. HDR can allow precise genetic modifications (mutations or corrections) in the target sequence. However, HDR typically occurs less frequently and is substantially more variable in frequency than NHEJ. The repair template can either be in the form of double-stranded DNA (a PCR product or a linearized plasmid), a targeting construct with homologous arms flanking both sides of an insertion/correction sequence, or a single-stranded DNA oligonucleotide (ssODN). A ssODN provides a simple and effective method for making small gene edits (<50 bp) within the genome, such as the introduction of single-nucleotide mutations for probing causal genetic variations. Unlike NHEJ, HDR is generally active only in dividing cells, and its efficiency can vary extensively depending on the cell type, as well as the genomic locus and the repair template.

A potential limitation of CRISPR nuclease systems is that they can cause off-target mutagenesis. To minimize off-target activity, several mutant forms of Cas9 protein have been developed to maximize introduction of DSBs at the desired target site. For example, a double nicking strategy can be used. The Cas9 protein (e.g., SpCas9) has two functional domains (RuvC and HNH), each cutting a different DNA strand. When both these domains are active, the Cas9 protein causes DSBs in the target DNA. Mutated versions of Cas9 that contain a single active catalytic domain, either RuvC or HNH, are known as nickases. For example, the RuvC domain can be inactivated by a D10A mutation and the HNH domain can be inactivated by an H840A mutation. Cas9 nickase (Cas9n) cuts only one strand of the target genomic DNA rather than both strands as with the wild type Cas9. The single-strand break or nick can be repaired without any indels at the target sequence using high-fidelity base excision repair (BER) pathways rather than NHEJ. While Cas9 protein induces DSB at the target sequence using a single gRNA, either RuvC⁻ or HNH⁻ mutant Cas9n requires a paired gRNA appropriately spaced and oriented to simultaneously introduce the single-stranded nicks on both strands of the target sequence (see FIGS. 1-3). Using Cas9n with a paired gRNA can reduce off-target effects by ˜50-1500 fold when compared to Cas9 nuclease activity with a single gRNA. Similar to Cas9 nuclease activity, DSB from Cas9n double nicks can be repaired by the cellular repair machinery, either through NHEJ or HDR, in the absence or presence of donor template, respectively. The single-stranded nicks are repaired by high-fidelity BER pathways without indel formation, as DSBs would only occur if both gRNAs were able to locate target sequences within a defined space. Thus, this strategy effectively doubles the number of bases that need to be specifically recognized at the target site and significantly increases the specificity of genome editing by several folds. Mutation on both RuvC⁻ or HNH⁻ catalytic domains results in double mutated Cas9 (dCas9) proteins, which cannot act on the target sequence to make DSB or nick. However, dCas9 retains the ability to bind to the target DNA sequence as directed by gRNA sequence. By their retention of binding ability to the target DNA sequence, dCas9 protein can be used for robust transcription activation or repression of downstream-targeted genes. Unlike the genome modifications induced by Cas9 and Cas9n, the dCas9 mediated transcription activation or repression of the target genes are reversible and does not induce permanent modifications at the target gDNA sequence. Thus by selecting an appropriate Cas9 nucleases (Cas9, Cas9n, and dCas9), gRNA(s) and donor template (in the case of HDR), the CRISPR/Cas system can be a remarkable and flexible tool for genome manipulation.

However, available CRISPR systems have several limitations such as low efficiency and low specificity, which leads to a low success rate and undesirable off-target mutagenesis. There is a need for improved CRISPR/Cas systems for genome editing in cells.

### SUMMARY

It is an object of the present invention to ameliorate at least some of the deficiencies present in the prior art. Embodiments of the present technology have been developed based on the inventors' appreciation that there is a need for improved CRISPR/Cas systems for targeted genomic modification and targeted regulation of gene expression in mammalian cells.

The present disclosure relates broadly to CRISPR/Cas systems having greater efficiency and/or specificity than other known CRISPR/Cas systems, and to uses thereof for targeted genomic modification within a target genome region (TGR) in a mammalian cell. Therapeutic uses of the methods and systems described herein are also provided.

In particular, there are provided modified CRISPR/Cas systems having in some embodiments one or more of the following advantages: higher efficiency of genomic modification; ability to more efficiently and/or safely transfect a CRISPR/Cas system multiple times; reduced off-site or non-specific modification (i.e., higher specificity of genomic modification); a higher efficiency of homology-directed repair (HDR); improved stability of a single-stranded DNA oligonucleotide (ssODN) HDR repair template; and/or other advantages as will become apparent herein.

In general, HDR occurs at a much lower frequency and is therefore less efficient than NHEJ in CRISPR/Cas9 gene editing systems. This low efficiency of HDR presents a major constraint in the execution of precise genetic modifications by the CRISPR/Cas9 system. In some embodiments, we provide herein HDR repair templates having an improved design that can improve stability of the ssODN HDR donor template and/or allow genetic modifications to be made more efficiently at a target site of interest. Modifications in an ssODN HDR repair template include, without limitation: adding a 4-nucleotide repeat (such as the CGCG repeat of phosphorothioate) to improve the stability of the ssODN HDR template; incorporating a peptide nucleic acid at the ssODN end (5′ or 3′) to increase efficiency of the HDR pathway; linking a Cyanine dye; and/or linking a quantum dot at the 5′ end of a ssODN to allow monitoring of its cellular uptake and/or distribution in a cell during genomic modification. Thus, stability, efficiency, and/or traceability of the repair template, e.g., a ssODN, may be improved.

Further, successful genome modification by Cas9 nuclease requires a PAM sequence at the 3′ end of the target genomic DNA, e.g., at the 3′ end of the 20-nucleotide target sequence to which the guide RNA binds. However, if an HDR template has an intact PAM sequence or retains an intact PAM sequence in the donor template after Cas9 modification has occurred, then Cas9 may repeatedly act on the target sequence, potentially leading to an increased chance of mutations and/or DNA degradation, even after the desired modification has been introduced. To avoid these unwanted activities by the CRISPR/Cas9 system, we provide herein, in some embodiments, modified CRISPR/Cas9 systems and repair templates in which the PAM sequence is mutated in the HDR repair template. For example, in the case of the SpCas9 enzyme, the PAM sequence “NGG” in the HDR template can be mutated to NGT, NGC or NGA. Such mutation will prevent binding by the Cas9 enzyme and thus “mask” the PAM sequence. It is noted that, where the HDR template sequence falls within a protein coding region (for example, in an exon or a promoter region), then care is taken to introduce a silent mutation in the PAM sequence to avoid introducing amino acid changes into the coding region. “Masking” a PAM sequence in this way means that an already-modified sequence will not be cut again by Cas9. Multiple transfections of the CRISPR/Cas9 system to increase efficiency are thus possible, as already-modified sequences will be unaffected.

In some embodiments, alternatively or additionally, further modification of an already-modified sequence is prevented by selecting one or more gRNA such that a mutation is introduced in a PAM sequence and/or a target DNA sequence, the introduced mutation preventing further modification by the CRISPR/Cas9 system. In such embodiments the “masking” mutation is introduced by one or more gRNA. The HDR repair template may or may not also introduce a “masking” mutation into the target genome region (TGR).

In a first broad aspect, there is provided a method for targeted genomic modification within a target genome region (TGR) in a mammalian cell, the method comprising providing a CRISPR/Cas9 system and contacting the mammalian cell with the CRISPR/Cas9 system, wherein the CRISPR/Cas9 system comprises: i) a first guide RNA (gRNA) comprising a first CRISPR RNA (crRNA) and a first trans-activating crRNA (tracrRNA) linked together, the first gRNA being capable of binding with sequence specificity to a first target DNA sequence on one strand of the DNA double helix in the TGR, the first target DNA sequence to which the first gRNA binds being adjacent to a first PAM sequence; ii) a second gRNA comprising a second CRISPR RNA (crRNA) and a second trans-activating crRNA (tracrRNA) linked together, the second gRNA being capable of binding with sequence specificity to a second target DNA sequence on the other strand of the DNA double helix in the TGR, the second target DNA sequence to which the second gRNA binds being adjacent to a second PAM sequence, wherein the first and the second target DNA sequence are located within about 100 to about 1000 nucleotides of each other, or within about 100 nucleotides of each other, and are on opposite strands of the DNA double helix; and iii) a Cas9n protein. In some embodiments, the first and the second target DNA sequence are located within about 100 nucleotides of each other. In some embodiments, the first and the second target DNA sequence are located within about 1000 nucleotides of each other. In some embodiments, the first and the second target DNA sequence are located more than 100 nucleotides from each other. The mammalian cell is contacted with the CRISPR/Cas9 system under conditions (sufficient time, etc.) such that the TGR is modified, forming a modified-TGR, the first and/or the second gRNA having been selected in some embodiments such that one or more of the first PAM sequence, the second PAM sequence, the first target DNA sequence and the second target DNA sequence are modified within the modified-TGR so as to prevent further modification of the modified-TGR by the CRISPR/Cas9 system.

In some embodiments, the CRISPR/Cas9 system further comprises: iv) a third gRNA comprising a third CRISPR RNA (crRNA) and a third trans-activating crRNA (tracrRNA) linked together, the third gRNA being capable of binding with sequence specificity to a third DNA sequence on one strand of the DNA double helix in the TGR, the third target DNA sequence to which the third gRNA binds being adjacent to a third PAM sequence; wherein the third target DNA sequence is located either within 100 nucleotides of the first target DNA sequence on the opposite strand of the DNA double helix or within 100 nucleotides of the second target DNA sequence on the opposite strand of the DNA double helix; and wherein the third gRNA is selected such that the CRISPR/Cas9 system can only bind and/or modify the third target DNA sequence if the target genome region comprises a disease-causing modification or a sequence for which modification is desired. By “wherein the third target DNA sequence is located either within 100 nucleotides of the first target DNA sequence on the opposite strand of the DNA double helix or within 100 nucleotides of the second target DNA sequence on the opposite strand of the DNA double helix” is meant that in some embodiments the separation between the first and the second or third target is about 100 nucleotides or is less than 100 nucleotides from each other, however it should be understood that in some embodiments greater separations of more than 100 nucleotides between target DNA sequences are possible.

In general, gRNAs and/or target DNA sequences can be appropriately spaced and oriented so that Cas9 will simultaneously introduce single-stranded nicks on both strands of the target sequence. If the single-stranded nicks on both strands of the target sequence are located sufficiently close together, then the cellular repair machinery will repair the nicks as a double stranded break (DSB) and introduce an indel mutation into the targeted DNA sequence. For example, in this case repair of the nicks via NHEJ will introduce an indel mutation into the targeted DNA sequence. In some embodiments therefore, where repair of DSB is desired, the gRNAs and/or target DNA sequences are selected so that the single-stranded nicks on both strands of the target sequence are located sufficiently close together to induce DSB repair. In some such embodiments, where DSB repair is desired, a first target DNA sequence on one strand of the DNA double helix and a second target DNA sequence on the opposite strand of the DNA double helix are separated by about 100 nucleotides or less than 100 nucleotides from each other. In some such embodiments, the first target DNA sequence on one strand of the DNA double helix and the second target DNA sequence on the opposite strand of the DNA double helix are separated by less than 100 nucleotides, by less than 50 nucleotides, by less than about 20 nucleotides, or by less than about 10 nucleotides. In alternate embodiments, where DSB repair is not desired and it is desired instead to have the cellular machinery simply repair the nicks made by Cas9, without introducing an indel mutation, then the first target DNA sequence on one strand of the DNA double helix and the second target DNA sequence on the opposite strand of the DNA double helix are located sufficiently far apart or separated by a sufficient number of nucleotides so that DSB repair does not occur; for example, they may be separated by more than 100 nucleotides from each other, to ensure no DSB repair and no introduction of an indel mutation.

In some embodiments, target DNA sequences on opposite strands of the DNA double helix are selected to be located within a certain distance of each other sufficient to induce double-stranded break (DSB) repair, e.g., to induce an indel mutation in the DNA. In alternate embodiments, target DNA sequences on opposite strands of the double helix are selected to be located within a certain distance of each other sufficient to not induce double-stranded break (DSB) repair, e.g., so that no genomic modification occurs; for example, nicks may be repaired without modifying the starting TGR or target DNA sequence.

In some embodiments, only one gRNA (e.g., only the first gRNA, the second gRNA, or the third gRNA) can bind to its target DNA sequence in a “normal” or non-disease causing TGR; in this case, the CRISPR/Cas9 system will only create a single nick in one strand of the double helix in the TGR. This nick will simply be repaired and will not modify the TGR or target DNA sequence. In some embodiments therefore, the CRISPR/Cas9 system can only bind and/or modify the TGR (e.g., the third target DNA sequence) in the mammalian cell of a patient suffering from a disease or in a mammalian cell where the TGR includes a disease-causing mutation.

In some embodiments, the third target DNA sequence is only adjacent to the third PAM sequence if the target genome region comprises a disease-causing mutation or a sequence for which modification is desired or is in the mammalian cell of a patient suffering from a disease. In some embodiments, one or more of the third PAM sequence and the third target DNA sequence are modified by one or more nucleotide change within the modified-TGR so as to prevent further modification by the CRISPR/Cas9 system, e.g., so that binding by the third gRNA and/or the Cas9n protein is prevented.

In some embodiments, the disease-causing mutation is a repeat expansion, e.g., a trinucleotide expansion, a hexanucleotide expansion, and the like. In some embodiments, the repeat expansion is at least about 30 bp long. For example, the repeat expansion may encompass 5 or more hexanucleotide repeats, 10 or more trinucleotide repeats, etc. In an embodiment, the repeat expansion encompasses more than 3 hexanucleotide repeats, more than 4 hexanucleotide repeats, or more than 5 hexanucleotide repeats. In some embodiments, the disease is a repeat expansion disorder, e.g., a trinucleotide repeat disorder such as without limitation Fragile X Syndrome, Huntington's disease, spinocerebellar ataxia, myotonic dystrophy, myoclonic epilepsy, and/or Friedreich's ataxia; a hexanucleotide repeat disorder, such as without limitation amyotrophic lateral sclerosis (ALS) and frontotemporal dementia; and the like.

In some embodiments, the disease-causing mutation is an amyotrophic lateral sclerosis (ALS)-causing mutation and/or the disease is ALS. In some embodiments, the disease-causing causing mutation is a Fragile X Syndrome-causing mutation and/or the disease is Fragile X Syndrome.

In some embodiments, where there are multiple gRNAs, the multiple gRNAs may be the same or different. For example, the first gRNA and the second gRNA may be the same or different from each other, and each may be the same or different from the third gRNA. Many such permutations are possible. Similarly, the first, second, and third PAM sequences may be the same or different.

In some embodiments, the CRISPR/Cas9 system may further comprise a repair template for homology-directed repair (HDR). The repair template may or may not comprise one or more nucleotide change in one or more of the first PAM sequence, the second PAM sequence, and the (optional) third PAM sequence, and/or one or more nucleotide change in one or more of the first target DNA sequence, the second target DNA sequence, and the (optional) third target DNA sequence. In some embodiments, the repair template is a single-stranded DNA oligonucleotide (ssODN). In some embodiments, the repair template further comprises a DNA sequence to be inserted or modified in the target genome region. In some embodiments, the repair template is capped at the 5′ end, the 3′ end, or both. The cap may comprise, for example, 4 nucleotides or a peptide linked to the repair template. The repair template may further comprise a tag at the 5′ end, the 3′ end, or both, e.g., a detectable moiety such as without limitation a fluorophore, a cyanine dye, or a quantum dot.

In some embodiments, the CRISPR/Cas9 system further comprises: v) a fourth gRNA comprising a fourth CRISPR RNA (crRNA) and a fourth trans-activating crRNA (tracrRNA) linked together, the fourth gRNA being capable of binding with sequence specificity to a fourth DNA sequence on one strand of the DNA double helix in the TGR, the fourth target DNA sequence to which the fourth gRNA binds being adjacent to a fourth PAM sequence; wherein the fourth target DNA sequence is located either within 100 nucleotides of the first target DNA sequence on the opposite strand of the DNA double helix or within 100 nucleotides of the second target DNA sequence on the opposite strand of the DNA double helix; and wherein the fourth gRNA is selected such that the CRISPR/Cas9 system can only bind and/or modify the fourth target DNA sequence if the target genome region comprises a disease-causing modification or a sequence for which modification is desired. By “wherein the fourth target DNA sequence is located either within 100 nucleotides of the first target DNA sequence on the opposite strand of the DNA double helix or within 100 nucleotides of the second target DNA sequence on the opposite strand of the DNA double helix” is meant that in some embodiments the separation between the first and the second, third or fourth target is about 100 nucleotides or is less than 100 nucleotides from each other, however it should be understood that in some embodiments greater separations of more than 100 nucleotides between target DNA sequences are possible.

In some embodiments, only one gRNA (e.g., only the first gRNA, the second gRNA, the third gRNA, or the fourth gRNA) can bind to its target DNA sequence in a “normal” or non-disease causing TGR; in this case, the CRISPR/Cas9 system will only create a single nick in one strand of the double helix in the TGR. This nick will simply be repaired and will not modify the TGR or target DNA sequence. In some embodiments therefore, the CRISPR/Cas9 system can only bind and/or modify the TGR (e.g., the third or fourth target DNA sequence) in the mammalian cell of a patient suffering from a disease or in a mammalian cell where the TGR includes a disease-causing mutation.

In some embodiments, the third target DNA sequence or the fourth target DNA sequence is only adjacent to the third PAM sequence or the fourth PAM sequence respectively if the target genome region comprises a disease-causing mutation or a sequence for which modification is desired or is in the mammalian cell of a patient suffering from a disease. In some embodiments, one or more of the third PAM sequence and the third target DNA sequence are modified by one or more nucleotide change within the modified-TGR so as to prevent further modification by the CRISPR/Cas9 system, e.g., so that binding by the third gRNA and/or the Cas9n protein is prevented. In some embodiments, one or more of the fourth PAM sequence and the fourth target DNA sequence are modified by one or more nucleotide change within the modified-TGR so as to prevent further modification by the CRISPR/Cas9 system, e.g., so that binding by the fourth gRNA and/or the Cas9n protein is prevented. In some embodiments, one or more of the third PAM sequence, the fourth PAM sequence, the third target DNA sequence, and the fourth target DNA sequence are modified by one or more nucleotide change within the modified-TGR so as to prevent further modification by the CRISPR/Cas9 system, e.g., so that binding by the third gRNA, the fourth gRNA and/or the Cas9n protein is prevented.

In a second broad aspect, there is provided a method for targeted genomic modification within a target genome region (TGR) in a mammalian cell, the method comprising providing a CRISPR/Cas9 system and contacting the mammalian cell with the CRISPR/Cas9 system, wherein the CRISPR/Cas9 system comprises: i) one or more guide RNA (gRNA) comprising a CRISPR RNA (crRNA) and a trans-activating crRNA (tracrRNA) linked together, the one or more gRNA being capable of binding with sequence specificity to a first target DNA sequence and a second target DNA sequence in the TGR, the first target DNA sequence to which the one or more gRNA binds being adjacent to a first PAM sequence, and the second target DNA sequence being adjacent to a second PAM sequence, wherein the first and the second target DNA sequence are located within 100 nucleotides of each other and are on opposite strands of the DNA double helix; ii) a Cas9n protein; and iii) a repair template for homology-directed repair (HDR), wherein the repair template comprises one or more nucleotide change in one or more of the first PAM sequence and the second PAM sequence. By “wherein the first and the second target DNA sequence are located within 100 nucleotides of each other” is meant that in some embodiments the separation between the first and the second target is about 100 nucleotides or is less than 100 nucleotides from each other, however in some embodiments greater separations of more than 100 nucleotides between target DNA sequences are possible. The mammalian cell is contacted with the CRISPR/Cas9 system under conditions such that the TGR is modified, forming a modified-TGR, the repair template having been selected such that one or more of the first PAM sequence, the second PAM sequence, the first target DNA sequence and the second target DNA sequence are modified within the modified-TGR so as to prevent further modification of the modified-TGR by the CRISPR/Cas9 system. In some embodiments, the repair template comprises a single nucleotide change in one or more of the first PAM sequence, the second PAM sequence, the first target DNA sequence, and the second target DNA sequence.

As used herein, a “target DNA sequence” is also referred to as a “target genomic DNA (gDNA) region”. These terms are used interchangeably when the target DNA sequence is in the genome of a mammalian cell.

In some embodiments, one or more of the PAM sequences and the target DNA sequences no longer exists in the genome of the mammalian cell, or exists only on one strand of the DNA double helix within the modified-TGR, such that the CRISPR/Cas9 system can no longer bind to and/or modify the modified-TGR. In an embodiment, one or more of the first gRNA, the second gRNA, and the Cas9n protein can't bind to at least one strand of the DNA double helix in the modified-TGR. In another embodiment, one or more of the first gRNA, the second gRNA, and the Cas9n protein can't bind to either strand of the DNA double helix in the modified-TGR.

Any combination of the PAM sequences and the target DNA sequences may be modified in the modified-TGR. For example, in some embodiments, only one of the first PAM sequence, the second PAM sequence, the first target DNA sequence, and the second target DNA sequence is modified in the modified-TGR. In other embodiments, two, three, or all of the first PAM sequence, the second PAM sequence, the first target DNA sequence, and the second target DNA sequence are modified in the modified-TGR. The number and location of such modifications is not particularly limited, as long as the CRISPR/Cas9 system can no longer bind to and/or modify the modified-TGR. In this way efficiency can be increased. Further, in some cases, multiple introductions of the CRISPR/Cas9 system into a cell are also made possible, as a modified-TGR in a cell will not be further cut or modified by Cas9.

A PAM sequence (e.g., a first PAM sequence, a second PAM sequence, etc.) may be any PAM sequence appropriate for the particular Cas9 protein being used. PAM sequences associated with the various Cas9 proteins as indicated are shown in Table 1. In an embodiment, the PAM sequence is selected from NGG, NNGRRT, NNGRRN, NNNNGATT, NNAGAAW, NAAAAC, NGG, NAG, NGCG, NGAG, NGAN, NGNG, and NTT, where R is A or G, W is A or T, and N is A, C, G, or T. In an embodiment, the PAM sequence is 3′-GGA-5′ and the modified-TGR comprises a single nucleotide change in the PAM sequence that changes the PAM sequence to 3′-TGA-5′. In an embodiment where a repair template is included, the repair template comprises a single nucleotide change in the PAM sequence that changes the PAM sequence to 3′-TGA-5′.

TABLE 1
Examples of Cas9 proteins and associated
PAM sequences.

| | Bacterial Species/ | PAM | |
|---|---|---|---|
| | Variant of Cas9 | Sequence** | Reference |
| 1. | Streptococcus | NGG | 1, 2 |
| | pyogenes(Sp)^{*} | | |
| 2. | Staphylococcus | NNGRRT or | 3 |
| | aureus(Sa)^{*} | NNGRRN | |
| 3. | Neisseria | NNNNGATT | 4 |
| | meningitides(Nm)^{*} | | |
| 4. | Streptococcus | NNAGAAW | 5 |
| | thermophilus(St)^{*} | | |
| 5. | Treponema denticola(Td)^{*} | NAAAAC | 6 |
| 6. | SpCas9 D1135E variant^{*} | NGG (reduced | 7 |
| | | NAG binding) | |
| 7. | SpCas9 VRER variant^{*} | NGCG | 7 |
| 8. | SpCas9 EQR variant^{*} | NGAG | 7 |
| 9. | SpCas9 VQR variant^{*} | NGAN or NGNG | 7 |
| 10. | Francisella | NTT | 8 |
| | novicida(Fn)^{#} | | |
| 11. | Neisseria cinerea(Nc)^{*} | NNNNGTA | 9 |
| 12. | Campylobacter | NNNNACAY | 9 |
| | jejuni(Cj)^{*} | | |
| 13. | More Cas9's from | Not yet | 10 |
| | various species | characterized | |

*Cas9 protein
^{#}Cpf1 protein
**R: A or G; W: A or T; N: A, C, G, or T
1 Mojica, F.J. et al., Microbiol. 155: 733-40, 2009.
2 Jinek, M.I. et al., Science 337(6096): 816-21, 2012.
3 Ran, F.A. et a., Nature 520(7546): 186-91, 2015).
4 Hou, Z. et al., Proc. Natl. Acad. Sci. USA 110(39): 15644-9, 2013.
5 Deveau, H. et al., J. Bacteriol. 190(4): 1390-400, 2008.
6 Cong, L. et al., Science 339: 819-23, 2013.
7 Kleinstiver, B.P. et al., Nature 523(7561): 481-5, 2015.
8 Zetsche, B. et al., Cell 163(3): 759-71, 2015.
9 Chen, F. et al., Nat Commun. 8: 14958, 2017.
10 Louwen, R. et al., Microbiol Mol Biol Rev. 78(1): 74-88, 2014.

In some embodiments, one or more nucleotide or base change is made in a PAM sequence in the modified-TGR in order to “mask” the PAM sequence so that binding by the Cas9 enzyme is prevented. In some embodiments, the mutation(s) introduced in the PAM sequence prevents binding of the Cas9n protein. In some embodiments, the mutation(s) introduced in the PAM sequence is silent, i.e., does not change the amino acid encoded by the sequence, if the PAM sequence is included in a protein coding region. In some embodiments, masking the PAM sequence by mutating it so that the Cas9 protein can no longer bind prevents modified target DNA sequences from being cut a second time by the Cas9 protein. In this way efficiency can be increased. Further, in some cases, multiple introductions of the CRISPR/Cas9 system into a cell are also made possible, as a modified target DNA sequence will not be further cut or modified by Cas9.

In some embodiments, a PAM sequence is partially or fully located in an intron in the TGR.

In some embodiments, a repair template is a single-stranded DNA oligonucleotide (ssODN). A repair template, e.g., ssODN, typically further comprises a DNA sequence to be inserted or modified in the target DNA sequence. In some embodiments, a repair template, e.g., ssODN, further comprises a cap at the 5′ end, the 3′ end, or both. A cap may be, without limitation, 4 nucleotides (such as a CGCG repeat), a peptide, or a detectable tag (such as a fluorophore or a dye, e.g., a Cyanine dye, or a quantum dot). One or more caps may be present on a repair template. In some embodiments, one or more cap may serve to increase stability of the repair template, increase efficiency of the HDR pathway, and/or allow monitoring of cellular uptake and/or distribution of the repair template in a cell.

In some embodiments the components of the CRISPR/Cas9 system are provided directly as nucleic acid (e.g., RNA, ssODN) and protein. In other embodiments the components are provided in the form of a DNA, such as a vector, that encodes the component, for expression in the mammalian cell. Each component may be encoded by the same DNA or by a different DNA. Further, some components may be provided directly (e.g., as an RNA or protein, etc.) while other components are provided in the form of a DNA. Many such permutations are possible and are not meant to be limited.

In one embodiment, one or more gRNA is provided by an episome (e.g., through an episomal vector) that encodes it. The mammalian cell may be contacted with the episome encoding the gRNA first, prior to contacting the mammalian cell with the Cas9n protein and/or the optional repair template. Without wishing to be bound by theory, it is believed that, in some embodiments, priming a cell with gRNA (by e.g. an episomal vector) in this way may increase efficiency of genomic modification by allowing higher and/or longer expression of the gRNA in the mammalian cell (due to replication of the episome in the cell as an example). It should be understood that the number of episomal vectors is not particularly limited. Multiple gRNAs may be provided on one or on multiple episomes, for example.

In one embodiment, the Cas9n protein is provided directly as an isolated protein. In another embodiment, the Cas9n protein is provided in the form of a nucleic acid, e.g., a DNA plasmid, encoding the Cas9n protein. In another embodiment, the nucleic acid encoding the Cas9n protein is an RNA.

In some embodiments, the CRISPR/Cas9 system is introduced into the mammalian cell via transfection. Other methods for introducing nucleic acids and proteins into cells are known and may be used. The method of introducing the CRISPR/Cas9 system into the cell is not meant to be particularly limited.

In some embodiments, the CRISPR/Cas9 system is introduced, e.g., transfected, into the mammalian cell more than once. Multiple transfections may be performed, as desired, to increase efficiency of genomic modification. For example, multiple transfections may represent two, three, five, ten, or more than ten transfections into the cell, either in vitro or in vivo or both. In some embodiments, the CRISPR/Cas9 system is introduced into the mammalian cell by a) first transfecting an epiosomal vector encoding the gRNA into the mammalian cell; and b) then transfecting the Cas9n protein or an RNA encoding the Cas9n protein and the repair template (if present) into the mammalian cell, the repair template (if present) being for example a ssODN, as described herein.

For illustrative purposes the Cas9n protein is used in the embodiments described herein. However, the Cas protein is not meant to be particularly limited. It should be expressly understood that any suitable Cas protein may be used in methods and systems provided herein. Further, the term “Cas9” protein is used herein to refer generally to different forms of the protein, such as without limitation Cas9 (wild-type), Cas9n, dCas9, and other appropriate modified versions of Cas9, unless specified otherwise.

The mammalian cell to be genomically-modified is also not particularly limited. Any suitable cell in which a genomic modification is desired may be used in methods provided herein. For example, a mammalian cell may be an embryonic stem cell, a pluripotent stem cell, an induced pluripotent stem cell, a multipotent stem cell, a directly reprogrammed multipotent stem cell, a precursor cell, a progenitor cell, or a somatic cell. A mammalian cell may be a neuronal cell (including neurons, astrocytes and oligodendrocytes), a neural progenitor cell, a neural precursor cell, a neural stem cell, as well as other somatic, precursor, progenitor and stem cells of ectodermal, endodermal and mesodermal lineage such as cells of cardiac lineage, blood lineage (except mature red blood cells that have no nucleus), muscle lineage, adipocyte (fat) lineage, epithelial lineage, endothelial lineage, epidermal lineage, pulmonary lineage, hepatic lineage, pancreatic lineage, as well as kidney and other organ and system lineages, as well as tumour cells and other abnormal cells, and the like. In an embodiment, the mammalian cell is a human cell.

In a third aspect, there is provided a method for treating or preventing ALS in a patient in need thereof, comprising use of the CRISPR/Cas9 systems provided herein to repair mutations in the SOD1 gene in the patient (e.g., to correct an H46R mutation, or to delete repeats). In some embodiments, methods provided herein may be used to repair mutations in the SOD1 gene in ALS patients. For example, the target gDNA region may include or be adjacent to an H46R mutation in the SOD1 gene. In an embodiment, the TGR comprises all or a portion of the DNA sequence set forth in region 31655770-31670821 of NCBI Reference Sequence NC_000021.9; the repair template is a ssODN having the sequence set forth in SEQ ID NO: 6 or 7; and/or one or more gRNA comprises the sequence set forth in SEQ ID NO: 4 or 5.

In some embodiments, one or more gRNA is selected such that the target gDNA region to which it binds is adjacent to the required PAM sequence only in the chromosome of an ALS patient carrying an ALS DNA mutation and not in a normal chromosome (that does not have the mutation, or has already been repaired by an earlier intervention). This ensures that genomic modification will only occur in the ALS patient on genomic DNA regions in need of repair.

In a fourth aspect, there is provided a method for treating or preventing HIV infection in a patient in need thereof, comprising use of the CRISPR/Cas9 systems provided herein to introduce mutations (e.g., deletions) in the CCR5 gene in the patient. In some embodiments, methods provided herein may be used to modify the CCR5 gene, e.g., the target gDNA region is in the CCR5 gene, includes the CCR5 gene, or is adjacent to the CCR5 gene. For example, the TGR may comprise all or a portion of the DNA sequence set forth in region 46372903-46373961 of NCBI Reference Sequence NC_000003.12; the gRNA may comprise the sequence set forth in SEQ ID NO: 8 or 9; and/or the repair template may be a ssODN having the sequence set forth in SEQ ID NO: 10 or 11.

In a fifth aspect, there is provided a method for treating cancer in a patient in need thereof, comprising use of the CRISPR/Cas9 systems provided herein to introduce mutations (e.g., deletions or insertions) into a cancer-causing gene (e.g., an oncogene) in the patient. In some embodiments, methods provided herein may be used to modify a cancer-causing gene (i.e., the TGR is in the cancer causing gene) to correct the gene so it is no longer cancer-causing. For example, the TGR may comprise all or a portion of the DNA sequence set forth in region 21967752-21995301 of NCBI Reference Sequence NC-000009. In other embodiments, methods provided herein may be used to modify one or more gene in a cancer cell, such as one or more gene that results in death of the cell, termination or reduction in proliferation and/or growth of the cell, and/or confers dependence on the presence or introduction (or the lack of presence) of a substance for continued survival of the cell.

In a sixth aspect, there is provided a method for treating or preventing ALS or Frontotemporal Dementia resulting from a mutated C9ORF72 gene in a patient in need thereof, comprising use of the CRISPR/Cas9 systems provided herein. In an embodiment, deletions are introduced in the mutated C9ORF72 gene in the patient. In some embodiments, such deletions are introduced without the use a repair template, e.g., without the use of a ssODN. In some embodiments, methods provided herein may be used to modify or correct a mutated C9ORF72 gene (i.e., the TGR is in the mutated C9ORF72 gene). In an embodiment, the TGR comprises all or a portion of the DNA sequence set forth in region 27546546-27573866 of NCBI Reference Sequence NC_000009.12; and/or one or more gRNA having the sequence set forth in any one of SEQ ID NO: 1, 2, and 3 is used. In a particular embodiment, three gRNAs having the sequences of SEQ ID NOs: 1, 2, and 3 are used.

In a seventh aspect, there is provided a method for treating or preventing a mitochondrial disease in a patient in need thereof, comprising carrying out targeted genomic modification within a target mitochondrial DNA region in a mammalian cell of the patient using methods provided herein. In an embodiment, the ssODN is conjugated with MSP or TPP and the target mitochondrial DNA region comprises the nt.A12770G mutation.

In an eighth aspect, there is provided a method for treating or preventing cystic fibrosis in a patient in need thereof, comprising carrying out targeted genomic modification within a target genome region (TGR) in a mammalian cell of the patient using methods provided herein. In an embodiment, the TGR comprises the W1282X mutation.

In a ninth aspect, there is provided a method for inactivation of a transgene in a genetically-modified organism (GMO), comprising carrying out targeted genomic modification within a target genome region (TGR) in a cell of the GMO using methods provided herein. The GMO may be a plant or an animal such as, without limitation, an α-interferon transgene-expressing genetically-engineered plant (such as a rice crop), a GFP-expressing transgenic fish, and the like.

In a tenth aspect, there is provide a method of treating a disease listed in Table 5 or Table 6 in a patient in need thereof, comprising carrying out targeted genomic modification within a target genome region (TGR) in a mammalian cell of the patient using methods provided herein.

In another aspect, there is provided a method for targeted genomic modification within a target genome region (TGR) in a mammalian cell, the method comprising: a) providing a CRISPR/Cas9 system comprising: i) a first guide RNA (gRNA) comprising a first CRISPR RNA (crRNA) and a first trans-activating crRNA (tracrRNA) linked together, the first gRNA being capable of binding with sequence specificity to a first target DNA sequence on one strand of the DNA double helix in the TGR, the first target DNA sequence to which the first gRNA binds being adjacent to a first PAM sequence; ii) a second gRNA comprising a second CRISPR RNA (crRNA) and a second trans-activating crRNA (tracrRNA) linked together, the second gRNA being capable of binding with sequence specificity to a second target DNA sequence, the second target DNA sequence to which the second gRNA binds being adjacent to a second PAM sequence, wherein the second target DNA sequence is on the same strand of the DNA double helix as the first target DNA sequence; iii) a third gRNA comprising a third CRISPR RNA (crRNA) and a third trans-activating crRNA (tracrRNA) linked together, the third gRNA being capable of binding with sequence specificity to a third DNA sequence on one strand of the DNA double helix in the TGR, the third target DNA sequence to which the third gRNA binds being adjacent to a third PAM sequence, wherein the third target DNA sequence is on the opposite strand of the DNA double helix from the first and the second target DNA sequences; wherein the first target DNA sequence and the second target DNA sequence are overlapping, such that the first gRNA and the second gRNA compete for binding to their respective target DNA sequences; and wherein at least one of the second gRNA and the third gRNA is selected such that the CRISPR/Cas9 system can only bind and/or modify the second target DNA sequence and/or the third target DNA sequence respectively if the target genome region comprises a disease-causing modification or a sequence for which modification is desired; and iv) a Cas9n protein; and b) contacting the mammalian cell with the CRISPR/Cas9 system such that the TGR is modified, forming a modified-TGR. In some embodiments, the CRISPR/Cas9 system can only bind and/or modify the second and/or the third target DNA sequence in the mammalian cell of a patient suffering from a disease. In some embodiments, this method is particularly useful for treating a disease-causing mutation which is a repeat expansion and/or for treating a repeat expansion disorder.

In another aspect, there is provided a method for treating or preventing a repeat expansion disorder, comprising carrying out targeted genomic modification within a target genome region (TGR) in a mammalian cell of the patient using methods provided herein. In an embodiment, the target genome region comprises a disease-causing mutation which is a repeat expansion, e.g., a trinucleotide expansion, a hexanucleotide expansion, and the like, and triple gRNA guided excision as described herein is used to remove extra repeats from the TGR. In other embodiments, the target genome region comprises a disease-causing mutation which is a repeat expansion, e.g., a trinucleotide expansion, a hexanucleotide expansion, and the like, and triple gRNA guided excision (e.g., using a gRNA1, a gRNA2 and a gRNA3 and as described herein, for example in FIG. 6) is used to remove extra repeats from the TGR.

In some embodiments, the repeat expansion is at least about 30 bp long. In some embodiments, the repeat expansion encompasses 5 or more hexanucleotide repeats, 10 or more trinucleotide repeats, more than 3 hexanucleotide repeats, more than 4 hexanucleotide repeats, or more than 5 hexanucleotide repeats. Any repeat expansion disorder may be treated using methods provided herein such as, without limitation, Fragile X Syndrome, Huntington's disease, spinocerebellar ataxia, myotonic dystrophy, myoclonic epilepsy, Friedreich's ataxia, amyotrophic lateral sclerosis (ALS) and/or frontotemporal dementia. In an embodiment, the disease-causing mutation is an amyotrophic lateral sclerosis (ALS)-causing mutation and/or the disease is ALS. In an embodiment, the disease-causing causing mutation is a Fragile X Syndrome-causing mutation and/or the disease is Fragile X Syndrome.

In another aspect, there is provided a method for targeted genomic modification within a target genome region (TGR) in a mammalian cell, the method comprising: a) providing a CRISPR/Cas9 system comprising: i) a first guide RNA (gRNA) comprising a first CRISPR RNA (crRNA) and a first trans-activating crRNA (tracrRNA) linked together, the first gRNA being capable of binding with sequence specificity to a first target DNA sequence on one strand of the DNA double helix in the TGR, the first target DNA sequence to which the first gRNA binds being adjacent to a first PAM sequence; ii) a second gRNA comprising a second CRISPR RNA (crRNA) and a second trans-activating crRNA (tracrRNA) linked together, the second gRNA being capable of binding with sequence specificity to a second target DNA sequence, the second target DNA sequence to which the second gRNA binds being adjacent to a second PAM sequence, wherein the second target DNA sequence is on the same strand of the DNA double helix as the first target DNA sequence; iii) a third gRNA comprising a third CRISPR RNA (crRNA) and a third trans-activating crRNA (tracrRNA) linked together, the third gRNA being capable of binding with sequence specificity to a third DNA sequence on one strand of the DNA double helix in the TGR, the third target DNA sequence to which the third gRNA binds being adjacent to a third PAM sequence, wherein the third target DNA sequence is on the opposite strand of the DNA double helix from the first and the second target DNA sequences; iv) a fourth gRNA comprising a fourth CRISPR RNA (crRNA) and a fourth trans-activating crRNA (tracrRNA) linked together, the fourth gRNA being capable of binding with sequence specificity to a fourth DNA sequence on one strand of the DNA double helix in the TGR, the fourth target DNA sequence to which the fourth gRNA binds being adjacent to a fourth PAM sequence, wherein the fourth target DNA sequence is on the opposite strand of the DNA double helix from the first and the second target DNA sequences; wherein at least one of the first gRNA, the second gRNA, the third gRNA, and the fourth gRNA is selected such that the CRISPR/Cas9 system can only bind and/or modify the respective target DNA sequence if the respective target DNA sequence comprises a disease-causing modification or a sequence for which modification is desired; and iv) a Cas9n protein; and b) contacting the mammalian cell with the CRISPR/Cas9 system such that the TGR is modified, forming a modified-TGR. In some embodiments, the CRISPR/Cas9 system can only bind and/or modify the respective target DNA sequence in the mammalian cell of a patient suffering from a disease. In some embodiments, the fourth gRNA is selected such that the CRISPR/Cas9 system can only bind and/or modify the fourth target DNA sequence if the fourth target DNA sequence comprises a disease-causing modification or a sequence for which modification is desired; and the second and the fourth target DNA sequence are located on opposite strands of the DNA double helix and are separated by a number of nucleotides sufficient to induce double stranded break (DSB) repair. In some embodiments, the second and the fourth target DNA sequence are separated by about 100 nucleotides or less than 100 nucleotides from each other, or by about 10 nucleotides or less, about 20 nucleotides or less, or about 50 nucleotides or less from each other. In some embodiments, the DSB repair introduces an indel mutation in the target genome region, e.g., knocking out or silencing the disease-causing modification in the target genome region.

In some embodiments, the third gRNA is also selected such that the CRISPR/Cas9 system can only bind and/or modify the third target DNA sequence if the third target DNA sequence comprises a disease-causing modification or a sequence for which modification is desired; in this case, the first and the third target DNA sequence are located on opposite strands of the DNA double helix and are separated by a number of nucleotides sufficient to induce double stranded break (DSB) repair (e.g., less than 100 nucleotides apart, less than 50 nucleotides apart, less than 20 nucleotides apart, or less than 10 nucleotides apart).

In alternative embodiments, the third gRNA and the first gRNA are selected such that the CRISPR/Cas9 system can bind and/or modify their respective target DNA sequences even if the respective target DNA sequences do not comprise a disease-causing modification; in this case, the first and the third target DNA sequence are located on opposite strands of the DNA double helix and are separated by a number of nucleotides sufficient to not induce double stranded break (DSB) repair (e.g., more than about 100 nucleotides apart).

In some embodiments, this method is particularly useful where the disease-causing mutation is a heterozygous mutation, e.g., a point mutation, e.g., a gain of function mutation, and only one chromosome requires repair. DSB repair is then only desired on the mutated chromosome. In an embodiment, the disease-causing mutation is a mutated SOD1 allele and/or the disease is ALS.

In another aspect, there is provided a method for treating or preventing a disease or condition caused by a heterozygous mutation, comprising carrying out targeted genomic modification within a target genome region (TGR) in a mammalian cell of the patient using methods provided herein. In an embodiment, the disease-causing heterozygous mutation is a point mutation. In some embodiments, the disease-causing heterozygous mutation is disrupted, i.e., removed or corrected. In other embodiments, the disease-causing heterozygous mutation is silenced or knocked out. It should be understood that in many cases where a disease-causing mutation is a heterologous mutation, knocking out the mutated allele can be as effective at treating the disease as correcting the gene mutation. For example, in many cases the non-mutated copy of the gene provides adequate levels of the gene product (protein or RNA), and the mutated copy of the gene interferes with the function of the wild-type gene product, thereby causing disease symptoms that would not occur if only the wild-type, non-mutated gene product were present. In such cases, knocking out the mutated allele provides a simpler strategy to treat the disease effectively and has a lower chance of introducing new, unwanted gene mutations during the genomic modification than repair of the mutated allele. In some embodiments therefore, there is provided a method for treating or preventing a disease or condition caused by a heterozygous mutation, comprising carrying out targeted genomic modification within a TGR in a mammalian cell of the patient to knock out the heterozygous mutation, using methods provided herein. In an embodiment, a heterozygous mutation is knocked out using a QuadPlex gRNA method as described herein (for example, in FIGS. 7E and 7F), i.e., using four gRNAs (gRNA1, gRNA2, gRNA3, and gRNA4). In an embodiment, the heterozygous mutation is a mutated SOD1 allele.

In another aspect, there are provided methods for highly efficient and precise targeted genomic modification within a target genome region (TGR, also referred to as gDNA). In some embodiments, delivery of the components of the targeted genomic modification system (e.g., Cas9, gRNAs, HDR templates if needed, etc.) into the cells carrying the TGR is enhanced by delivering the targeted genomic modification components on a plasmid into the cells, and optionally selecting for transfected cells carrying the plasmid. For example, in some embodiments, Cas9 or Cas9n is delivered as an episomal or non-episomal plasmid expressing the Cas9 or Cas9n mRNA or protein. In some embodiments, CRISPR gRNAs are delivered in combination with Cas9 or Cas9n as episomal or non-episomal plasmids. Further, in some embodiments, gRNAs produced using in vitro transcription (also referred to as “IVT gRNAs”) are pre-loaded onto a Cas9 protein. In some embodiments, cleavage specificity of the CRISPR/Cas9 system is further enhanced by direct introduction into a cell of a pre-complexed Cas9 or Cas9n protein with IVT gRNA to form a Cas9/gRNA ribonucleoprotein complex (Cas9/gRNA RNP). This approach can be fine-tuned to deliver the Cas9/Cas9n protein at the optimum concentration to limit off-target cleavage by utilizing a cell's own endogenous degradation machinery to rapidly degrade the Cas9 or Cas9n protein, once it has completed its activity.

In another aspect, there are provided methods for highly efficient and precise targeted genomic modification within a target genome region in a population of cells comprising introducing components of the targeted genomic modification system into the cells using an episomal plasmid and selecting for transfected cells having the episomal plasmid therein. In some embodiments, transfected cells are selected by screening for truncated proteins and/or tag-epitopes that are encoded by the episomal plasmid encoding the components of the targeted genomic modification system (Cas9, gRNAs, etc.) and expressed at the cell surface of transfected cells carrying the episomal plasmid. For example, in some embodiments an episomal plasmid encoding one or more gRNA and/or Cas9 also encodes a truncated surface protein or a protein that confers specific antibiotic resistance to a cell, allowing for selection and purification of transfected cells carrying the episomal plasmid using, e.g., sorting, magnetic antibody separation, a specific antibody for the truncated surface protein, and the like. Cells having the episomal plasmid are then selected and purified out of the starting cell population, greatly enriching the number of genomic-modified cells in the population. In some embodiments, a completely pure or nearly pure genomic-modified cell population may be obtained using an episomal plasmid. It should be understood that the components of the targeted genomic modification system (Cas9, gRNAs, etc.) are generally encoded by the same episomal plasmid encoding the truncated protein and/or tag-epitope. However, in alternate embodiments, the components of the targeted genomic modification system (Cas9, gRNAs, etc.) and the truncated protein and/or tag epitope may be encoded by separate episomal plasmids which are co-transfected into the cells.

In some embodiments, transfected cells are selected and/or purified without antibiotic selection by transfecting an episomal plasmid encoding a non-immunogenic N- or C-terminal truncated protein that is expressed at the cell surface of transfected cells carrying the episomal plasmid. This approach can be utilized for any cell type, whether cells are adherent or grow in suspension, for rapid antibiotic-free selection of transfected cells, in order to enrich the percentage of genomic-modified cells. In some embodiments, an episomal plasmid encoding a non-immunogenic N- or C-terminal truncated protein may also encode a tag-epitope. Alternatively, an episomal plasmid encoding a tag-epitope may be used. A tag-epitope can be used similarly either in place of or in addition to a truncated surface protein as a selection, tracking and purification tool for transfected cells. In some embodiments, tag-epitopes are inserted between the ends of an outer membrane signal peptide and before the start codon of a truncated protein. It should be understood that any suitable truncated protein and/or tag-epitope may be used.

Exemplary gRNA and repair template sequences are shown in Table 2 and Table 6. In another aspect, there are provided isolated nucleic acids comprising or consisting of any one of the sequences set forth in SEQ ID NOs: 1-103, 112 and 113.

TABLE 2
Exemplary gRNA and repair template sequences.

| | | SEQ |
|---|---|---|
| | | ID |
| Description | Sequence(5′ → 3′)¹ | NO: |
| gRNA1 in | 5′-GAGTCGCGCGCTAGGGGCCGGGG-3′ | 1 |
| FIG. 6 | | |
| gRNA2 in | 5′-CCGGGGCCGGGGCCGGGGCG TGG-3′ | 2 |
| FIG. 6 | | |
| gRNA3 in | 5′-CCCGGCCCCGGCCCCGGCCCCGG-3′ | 3 |
| FIG. 6 | | |
| gRNA1 in | 3′-GGACGTACCTAAG [symbol] CAAGTAC-5′ | 4 |
| FIG. 7 | | |
| gRNA2 in | 5′-TTGGAGATAATACAGCAGGTGGG-3′ | 5 |
| FIG. 7 | | |
| Sense | 5′-ggtg . . . | |
| strand HDR | tgaagg [symbol] ctgcatggattc [symbol] gttcatgagtttggagataatacagcaggtgg [symbol] tg | 6 |
| template in | ttgt . . . ccct-3′ | |
| FIG. 7 | | |
| Anti-sense | 3′ccac . . . | 7 |
| strand HDR | acttcc [symbol] gacgtacctaag [symbol] caagtactcaaacctctattatgtcgtccacc [symbol] aca | |
| template in | aca . . . ggga-5′ | |
| FIG. 7 | | |
| gRNA1 in | 3′-ggtatgt [symbol] [symbol] -5′ | 8 |
| FIG. 8 | | |
| gRNA2 in | 5′-aaagatagtcatcttggggctgg-3′ | 9 |
| FIG. 8 | | |
| Sense | 5′-gaaggtcttcattacacctgcagctctcattttccatacattaaagatagtcatcttggggct [symbol] | 10 |
| strand HDR | gtcctgccgctgcttgtcatggtcatctgctactcgggaatcct-3′ | |
| template in | | |
| FIG. 8 | | |
| Anti-sense | 3′ . . . cttccagaagtaatgtggacgtcgagagtaaaaggt [symbol] aatttctatcagtagaa | 11 |
| strand HDR | ccccgaccaggacgagga . . . -5′ | |
| template in | | |
| FIG. 8 | | |
| gRNA1 in | 3′-GGTATGATTAGAATCAATGGCGA-5′ | 80 |
| FIG. 10 | | |
| gRNA2 in | 5′-TTCCAACTGTT CAT CGGCTGAGG-3′ | 81 |
| FIG. 10 | | |
| Sense | 5′- . . . atat . . . cttcctaattacTatGctTatcttGgttaccgctaacaacctat | 82 |
| strand HDR | tccaactgttcatcggAtgGg [symbol] gggcgtagga . . . tcat . . . -3′ | |
| template in | | |
| FIG. 10 | | |
| Anti-sense | 3′- . . . tata . . . gaaggattaatgAtaCgaAtagaaCcaatggcgattgttggataagg | 83 |
| strand HDR | ttgacaagtagccTacCc [symbol] cccgcatcct . . . agta . . . -5′ | |
| template in | | |
| FIG. 10 | | |
| gRNA1 in | 3′-GGTCCTGGAGCCGCACCGGATCG-5′ | 84 |
| FIG. 11 | | |
| gRNA in | 5′-AGTTATGGCGACGAAGGCCGTGG-3′ | 85 |
| FIG. 11 | | |
| gRNA1 in | 3′-GGAGGCTCCGCC TGTCG TGCCTG-5′ | 86 |
| FIG. 12 | | |
| gRNA2 in | 5′-TGGGGCCGCAGGGCGTGGATGGG-3′ | 87 |
| FIG. 12 | | |
| gRNA1 in | 3′-GGTTGAGTTTACATTTTAAATAC-5′ | 88 |
| FIG. 13 | | |
| gRNA2 in | 5′-GATGATATAACAAATCAATATGG-3′ | 89 |
| FIG. 13 | | |
| gRNA1 in | 3′-GGCAAATAATCTTTTAGTTAGAG-5′ | 90 |
| FIG. 14 | | |
| gRNA2 in | 5′-CCGCCGATATATTACAGAACAGG-3′ | 91 |
| FIG. 14 | | |
| gRNA1 in | 3′-GGACGAGCACAATGTCCGCCCCA-5′ | 92 |
| FIG. 15 | | |
| gRNA2 in | 5′-TACCACAGAGTCTAGACTCGTGG-3′ | 93 |
| FIG. 15 | | |
| gRNA1 in | 3′-GGAGTTACGATAAGTTGT ATTTG-5′ | 94 |
| FIG. 16 | | |
| gRNA2 in | 5′-AACCGAGTCCGATGAAAAAAAGG-3′ | 95 |
| FIG. 16 | | |
| gRNA1 in | 3′-GGAGACGTTCTTCGCGGGCTTCG-5′ | 96 |
| FIG. 17 | | |
| gRNA2 in | 5′-CTGGGGGCAGCCGATACCCGGGG-3′ | 97 |
| FIG. 17 | | |
| gRNA1 in | 3′-GGGTTAAGAACAACTTAATCTAC-5′ | 98 |
| FIG. 18 | | |
| gRNA2 in | 5′-GGGCAAAAATT CTCTGTCAGTGG-3′ | 99 |
| FIG. 18 | | |
| gRNA1 in | 3′-GGTGTCGGAGCCATTATCCTCCC-5′ | 100 |
| FIG. 19 | | |
| gRNA2 in | 5′-TTGATACTCCTGGGACAAATGGG-3′ | 101 |
| FIG. 19 | | |
| gRNA1 in | 3′-GGTCTAGCTACCACACAGAACCC-5′ | 102 |
| FIG. 20 | | |
| gRNA2 in | 5′-CAATAACTTTGCAACAGTGAAGG-3′ | 103 |
| FIG. 20 | | |
| gRNA1 in | 5′-GGCATTCGTGGATGGCGATC-3′ | 112 |
| FIG. 22 | | |
| gRNA2 in | 5′-ACCAATTCAGGGACCAGCGC-3′ | 113 |
| FIG. 22 | | |

¹PAM sequences are single underlined; Bold and double underlined bases represent modifications introduced into the modified-TGR by the CRISPR/Cas9 system.

Therapeutic application of methods and systems provided herein is not meant to be particularly limited. For example, methods and systems may be used for genetic modification, for gene-editing, to manipulate DNA in a cell, to increase or decrease expression of a particular gene, to correct mutated sequences or base pairs, to correct deletions or insertions of sequences, to cause deletions or insertions of sequences, to induce mutations, to inactivate a transgene in a genetically modified organism, and the like. Methods and systems may thus be used in a wide range of applications, including therapeutically in a patient, e.g, a human patient, or in a cell derived from or isolated from a human patient. Methods and systems provided herein may be used ex vivo or in vivo.

In another aspect, there is provided a repair template for HDR, e.g., a ssODN, as described herein. For example, isolated ssODNs having modifications as described above are provided (e.g., having mutated PAM sequences, capped ends, etc). Further, DNAs such as recombinant vectors encoding and expressing such ssODNs are also provided. In an embodiment, an ssODN having the sequence set forth in SEQ ID NOs: 6, 7, 10, 11, 82, or 83 is provided. DNAs such as recombinant vectors encoding and expressing such ssODNs are also provided.

In yet another aspect, there is provided a guide RNA for treating ALS or HIV, having the sequence set forth in SEQ ID NOs: 1, 2, 3, 4, 5, 8, or 9. DNAs such as recombinant vectors encoding and expressing such gRNAs are also provided.

In still another aspect, there are provided cells comprising repair templates, e.g., ssODNs, and gRNAs described herein, as well as DNAs encoding them. In yet another aspect, there are provided cells comprising a CRISPR/Cas9 system described herein.

In a further aspect, there is provided a kit for genomic modification in a cell. The kit may comprise a repair template, e.g., an ssODN, a gRNA, and/or a Cas9 protein, or nucleic acids encoding them, and instructions for use thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to embodiments of the present invention, and in which:

FIG. 1 is a schematic drawing illustrating components of the CRISPR/Cas9 system. (A): Cas9 protein (Cas9); CRISPR RNA (crRNA); trans-activating crRNA (tracrRNA); and target genomic DNA (gDNA) region are shown. (B): A guide RNA (gRNA) including the tracrRNA attached to the crRNA is shown. A gene specific 20-nucleotide guide sequence inserted in the gRNA is sufficient to direct the Cas9 protein to that specific gene's target sequence in the genome to execute a gene-editing process.

FIG. 2 is a schematic drawing illustrating that a PAM sequence is required for Cas9 recognition in the target genome sequence. (A) shows the importance of the PAM sequence for Cas9 protein to recognize the target sequence in the genome. (B) illustrates that the absence of the PAM sequence prevents the binding of Cas9 to its target.

FIG. 3 is a schematic drawing illustrating delivery of Cas9/Cas9n with the appropriate gRNA-induced double stranded break (DSB) in the target DNA sequence in the genome. (A): By delivering the Cas9 plasmid or protein with appropriate gRNAs into a cell, Cas9 can be utilized to cleave the gDNA towards almost any desired location. Cas9 protein induces DSB at the target sequence using a single gRNA. (B): Either RuvC⁻ or HNH⁻ mutant Cas9n requires a pair of gRNAs appropriately spaced and oriented to simultaneously introduce single-stranded nicks on both strands of the target sequence. (C): Schematic illustration showing multiple strategies for precise and efficient modification of gDNA regions using CRISPR/Cas9 technology. A typical CRISPR-Cas9 mediated genome editing requires two essential components: Cas9 or Cas9n protein and gRNA. Additionally, in the case of HDR-mediated genome editing a donor template will be required. A major challenge for effective precision gene editing is delivery of the above components of CRISPR/Cas9 system into the target cells. To address this problem, several targeting approaches can be used to deliver Cas9, gRNAs and HDR templates to achieve high efficiency gene editing, including: i) Cas9 or modified versions of Cas9 including Cas9n and dCas9 can be delivered as an episomal or non-episomal plasmid, Cas9 or Cas9n mRNA or as the active protein, and ii) CRISPR gRNAs can be delivered in combination with Cas9 or Cas9n as episomal or non-episomal plasmids, and IVT gRNAs can be optionally pre-loaded into a Cas9 protein (Cas9 is used interchangeably within the application to mean Cas9, Cas9n, dCas9, or other appropriate modified versions of Cas9). In order to introduce a DSB, the cas9 enzyme can be directed to a particular gDNA using a gRNA and PAM sequence in the target gDNA region. If the purpose of the genome editing is to introduce specific modifications into the gDNA sequences, the Cas9/gRNA complex requires a donor template, which can help utilize the cell's intrinsic HDR pathway to introduce these modifications. However, in the case of introducing an indel mutation into the targeted DNA sequence to knockout a particular gene, no donor template is required. Cleavage specificity of the CRISPR/Cas9 system can be further improved by the direct introduction into a cell of a pre-complexed Cas9 or Cas9n protein with IVT gRNA to form a Cas9/gRNA ribonucleoprotein complex (Cas9/gRNA RNP). This approach can be fine-tuned to deliver the Cas9/Cas9n protein at the optimum concentration to limit off-target cleavage by utilizing a cell's own endogenous degradation machinery to rapidly degrade the Cas9 or Cas9n protein, once it has completed its activity.

FIG. 4 is a schematic drawing illustrating plasmid constructs for gene editing therapeutic applications.

FIG. 5 is a schematic drawing illustrating structure of gRNA expression cassettes for gene editing therapeutic applications. (A): Structure of a single gRNA expression plasmid constructed with the human U6 promoter is shown; (B): structure of a single gRNA expression plasmid constructed with the human H1 promoter is shown; (C): structure of a multiplex gRNA expression plasmid with both the human U6 and H1 promoters is shown.

FIG. 6 is a schematic drawing illustrating triple gRNA guided excision of extra GGGGCC hexanucleotide repeats in the C9ORF72 gene. gRNA1, gRNA2 and gRNA3 are exclusively designed to bind only with ALS-genomic DNA. (A): In normal human genomic DNA that has three or fewer GGGGCC hexanucleotide repeats, gRNA competitively binds at the target sites, which results in binding of either gRNA1 or gRNA2. Due to the absence of a PAM sequence in the lower strand, gRNA3 will not support binding of Cas9 in the target C9ORF72 gene. Among the three gRNAs only one of them (either gRNA1 or gRNA2) can bind at the target site and can make a single DNA nick, which will be repaired by a high-fidelity BER pathway, without any genetic scarring. (B, C): However, the triple gRNAs can efficiently bind to the ALS patient's genomic DNA, which has more than 5 repeats of the GGGGCC hexanucleotide. gRNA1, gRNA2 and gRNA3 can execute the excision of extra GGGGCC hexanucleotide repeats from the ALS C9ORF72 gene. This induces a double stranded break (DSB), which will be repaired by a cellular NHEJ pathway. (D): PCR assay showing the results of triple gRNA guided excision of extra GGGGCC hexanucleotide repeats in the C9ORF72 gene in an ALS patient's derived neural stem-like cells (ALS-NSLC), compared to untransfected ALS-NSLC control cells. The PCR assay was performed using the forward primer NWL-MBPr-664 (5′-GGGTCTAGCAAGAGCAGGTGTGGGTTTAGGAGGTGTGTG-3′)(SEQ ID NO: 104) and the reverse primer NWL-MBPr-674 (5′-GCCCCGACCACGCCCCGGCCCCGGCCCCGGCCCCTAGCG-3′) (SEQ ID NO: 105). The patient's NSLC cell population was transfected with a pD-Epi723gRNA1 plasmid (SEQ ID NO: 106; shown schematically in FIG. 24) followed by transfection of Cas9 mRNA, and using no antibiotic selection or any other type of cell selection to purify the cells. In wild type gDNA, the PCR results showed a high-intensity band (Lane-1) and no band or a low-intensity band for the unedited ALS-NSLC (Lane-2). The edited ALS-NSLC gDNA (Lane-3) showed a high-intensity band at ˜211 bp which was comparable to the normal gDNA band's intensity (Lane-1). The PCR products obtained showed that CRISPR/Cas9 and triple gRNA based gene editing effectively excised the extra GGGGCC hexanucleotide repeats in the mutated ALS-C9ORF72 gene. The samples were run on a 1.5% agarose gel.

FIG. 7 is a schematic drawing illustrating correction of H46R mutation targets in the SOD1 gene for ALS gene therapy. Dual gRNA (A) and single stranded ODN HDR template (B) are designed for correcting the H46R mutation in the SOD1 gene. Sequences underlined in (B) are the selected gRNAs that guide Cas9n to create nicks on both strands of the SOD1 gene. The DSB in the SOD1 gene target is then precisely repaired by a HDR pathway using a sense or anti-sense ssODN HDR template that contains a corrected histidine codon at the 46th position and a masked PAM sequence. The PAM sequence in the ssODN is masked by point mutations. The ssODN used for SOD1 H46R genome modification is an example of hypothetical correction of mutations in both exon and intron regions as the 5′ end and 3′ end PAM sequences are presented in exon and intron regions, respectively. The 5′ exon PAM sequence, NGG is masked with NGT without changing any amino acid codons, however such caution is not required at the intronic 3′ PAM sequence as it can be masked with NGT, NGC and NGA. Thus in addition to precisely repairing the SOD1 gene, a masking mutation in the PAM sequence is introduced. (C): The mutation in the PAM sequence inhibits Cas9n binding to the repaired target sequence. (D): Schematic illustration of an HDR mediated gene-editing strategy for H46R correction using multiple HDR-plasmid transfections is shown. During the HDR repair process, it is possible that the cellular machinery may introduce intrinsic corrections to restore the histidine codon (H) and it also possible that indels may be formed by NHEJ. Indels could cause frame shift mutations in the targeted gene that may result in mRNA degradation by nonsense mediated decay or production of truncated non-functional proteins. The corrected cells express normal SOD1 gene and the untouched cells express H46R mutated SOD1 protein that aggregates in neural cells and causes ALS disease. The strategy attempts to introduce HDR plasmid through several rounds of transfections along with corrective HDR donor template to repair the H46R mutation in the majority of cells. Indel mutations will result in deactivation of SOD1 protein synthesis, which would decrease or arrest mutant SOD1 protein aggregation in the cells. Hence, even though there is a possibility of Indel mutations by this strategy, the knockout of the gene is not lethal or has no deleterious effect, and is most likely beneficial compared to the mutated SOD1 gene expression that leads to ALS. Furthermore, since the design of the ssODN and gRNA used here ensures that already corrected cells are not further affected, every attempt of transfecting HDR-plasmid with the corrective HDR template can significantly increase the number of corrected cells. Moreover, the repeated attempts of HDR repair can repair the Indel mutations too. The strategy thus allows for restoration of protein expression efficiently and safely by HDR mediated genome editing. This same methodology can be applied to treat similar gene mutations in other genes or in other locations in the genome. (E, F): Schematic diagram illustrating the different mutated allele-specific gene editing strategies to inactivate the mutated SOD1 gene. As described in FIG. 7D, the knockout of the mutated SOD1 allele has no significant deleterious effects, and its knockout is beneficial compared to mutated SOD1 gene expression that produces aggregated mutated SOD1 protein causing ALS. In order to target the mutated SOD1 allele, a QuadPlex gRNA strategy is used: four gRNAs are introduced into the cell, with one of the gRNAs being unique to the mutated SOD1 allele, and the distance between each gRNA being adjusted to achieve allele specific SOD1 gene knockout. Such QuadPlex gRNA provides a wide range of options for making one of the four gRNAs mutation specific, and adjusting the distance between each gRNA of the QuadPlex designs for each gRNA location provides allele specific knockout of mutated genes. This design is shown in FIG. 7E (where the mutation-specific gRNA is highlighted in red color). In the presence of the mutation, the CRISPR/Cas9 system with the QuadPlex gRNAs induces the two DSBs resulting in removal of a gDNA region, to knockout the mutated SOD1 gene; whereas in the normal SOD1 gene allele, only three gRNAs bind forming a nick and the removal of small ssDNA fragments that are repaired without any indels at the target sequence with the cell's standard high-fidelity base excision repair pathways rather than NHEJ (as shown in FIG. 7F). The same methodology can be applied to treat similar gene mutations in other target genes or other gene locations in the genome or in mitochondrial DNA.

FIG. 8 is a schematic drawing illustrating gene editing strategies to eradicate HIV infection and replication. (A-C): Excision of the CCR5432 sequence using CRISPR/Cas9 system to generate HIV resistance in cells is shown. CCR5432 mutation can be advantageous by preventing CD4-mediated entry of HIV and provide resistance to HIV infection. Dual gRNA (A) and single stranded ODN HDR template (B) were designed for deletion of 32 base pairs in the CCR5 gene. The underlined sequences in (B) are the selected gRNAs that guide Cas9n to create nicks on both strands of the CCR5 gene. The DSB in the CCR5 gene target is then repaired by an HDR pathway using a sense or anti-sense ssODN HDR template, precisely excising the 32 base pair sequence. This deletion of 32 base pairs also precisely removes 16 bp of the gRNA1 sequence, preventing gRNA1 and Cas9n from binding to the target sequence. However, existence of gRNA2 sequence in the CCR5 gene would lead to repeated nicking by gRNA2 and Cas9n. To avoid this unwanted activity in the CCR5432 mutated target, the PAM sequence for gRNA2 is masked silently (i.e., without changing the amino acid codon). (C): The resulting CCR5432 mutation no longer functions as a co-receptor for CD4 and prevents HIV entry into cells. The mutated PAM sequence inhibits Cas9n binding to the target. (D): Another strategy involving inactivation of HIV proviral gene expression to inhibit latent HIV replication and maturation is illustrated. An HIV protease knockout plasmid is designed that expresses Cas9N enzyme and gRNAs that specifically bind to the gene for HIV protease to eliminate expression of the integrated HIV genome. This strategy specifically knocks out HIV protease expression by indel formation that results in a frame shift in the HIV protease gene (E), resulting in a non-functional protease protein. The cells that lack HIV protease cannot produce mature infectious HIV viral particles.

FIG. 9 shows a schematic diagram of the structure of ssODN donor templates.

FIG. 10 is a schematic drawing illustrating HDR mediated genome editing for mitochondrial disease. (A): Illustration of a HDR mediated mitochondrial DNA-editing strategy that requires MTS-Cas9 plasmid and corrective HDR donor conjugated with PNA-MSP or PNA-TPP targeting the disease-causing mitochondrial mutation (in this case MELAS disease caused by a nucleotide nt.A12770G mutation). Due to the complexity of the mitochondrial DNA location, certain modifications that target the gene editing tools towards mitochondria are required in the nuclear CRISPR/Cas9 mediated gene editing methodology to precisely edit the mutated mitochondrial DNA. The localized oxidative environment and increased replication sometimes make mitrochondrial DNA mutation more frequent; in such conditions, mutant mitochondrial DNA co-exist with normal or wild type DNA in various proportions, which is called heteroplasmy. Thus a sufficiently high proportion of mutant mitochondrial DNA must be present for the disease to be expressed or to increase its severity. The illustrated design selectively repairs the mutant mitochondrial DNA, increasing the proportion of wild type mitochondrial DNA until completely recovered from the disease phenotype. (B): Design of two gRNAs that exclusively bind to the nt.A12770G mutated region in the mitochondrial DNA to correct the nt.A12770G mutation and restore mitochondrial function. Introducing an ssODN HDR donor template, either sense or anti-sense, induces HDR cellular machinery to join and repair the DSB. The PAM sequence and the 3′ end of the gRNA sequences are masked without changing the amino acid codon to avoid unwanted further activity by the CRISPR/Cas9 system in mitochondria where the mitochondrial DNA sequence has already been corrected.

FIG. 11 is a schematic drawing illustrating complete functional deletion of pathogenic mutant gene expression by Indel mediated gene editing. (A): Gene knockout methodology by NHEJ-mediated gene editing to silence the expression of mutated SOD1 protein that aggregates in neural cells and causes ALS disease. During the NHEJ repair process, cellular machinery can introduce insertion or deletion of a few base pairs in the SOD1 gene that could cause frame shift mutations in the targeted gene and lead to mRNA degradation by nonsense-mediated decay or result in the production of truncated non-functional proteins. NHEJ-mediated indels suppress toxic SOD1 production and aggregation in cells, especially neural cells. (B-C): Design of two gRNAs that exclusively bind to the first exon of the mutated SOD1 gene to completely inhibit the expression of the pathogenic (mutated) SOD1 gene, while leaving the non-mutated SOD1 gene (in the other chromosome in each cell) intact. Absence of a HDR donor activates the NHEJ-mediated DNA repair pathway that directly rejoins the two ends in the DSB site with the insertion or deletion of a few base pairs. Indels formed during the NHEJ repair process permanently inhibit mutated SOD1 gene expression by frame shift mutation in the promoter region.

FIG. 12 is a schematic drawing illustrating knock-out of a gene required for survival of a virus that affects neurons. The diagram shows the HSV viral sequence targeted in the ICP0 gene. Cas9n recruitment to the ICP0 gene is mediated by dual gRNAs that contain a 20 bp protospacer recognizing sequence (in green) followed by a PAM sequence (in red). Gathering of Cas9n and gRNAs induces a double stranded break (DSB) in the target region. The absence of a HDR donor template activates NHEJ repair to join the DSB in the target sequence. The “N” and “X” indicated in the NHEJ repaired target sequences denotes insertion or deletion of a few base pairs during DSB repair. Indels formed during the NHEJ repair process result in a frame shift mutation in the viral genome that results in the formation of a non-functional ICP0 protein. This in return may permanently halt HSV replication and completely eradicate HSV infection as ICP0 plays a critical role in the HSV life cycle by stimulating the onset of HSV lytic infection and productive reactivation of HSV viral genomes from latency.

FIG. 13 is a schematic drawing illustrating knock-out of a gene required for survival of a human parasite. The diagram shows the clag3 gene sequence of the Plasmodium falciparum (P. falciparum) genome. Cas9n recruitment to the clag3 gene is mediated by dual gRNAs that contain a 20 bp protospacer recognizing sequence (in green) followed by a PAM sequence (in red). Gathering of Cas9n and gRNAs induces a double stranded break (DSB) in the target region. Absence of a HDR donor template activates NHEJ repair to join the DSB. The “N” and “X” indicated in the NHEJ repaired target sequences denotes insertion or deletion of a few base pairs during DSB repair. Indels formed during the NHEJ repair process result in a frame shift mutation that results in the formation of a non-functional clag3 protein. This in return may permanently halt P. falciparum growth and proliferation as Clag3 plays a critical role in the determination of channel mediated nutrient uptake by infected red blood cells for the growth and proliferation of plasmodium parasites.

FIG. 14 is a schematic diagram illustrating knock-out of a gene required for survival of a pathogenic bacteria that affect humans. The diagram shows the SigI gene sequence of the Bacillus anthracis genome (causing Anthrax). Cas9n recruitment to the SigI gene is mediated by dual gRNAs that contain a 20 bp protospacer recognizing sequence (in green) followed by a PAM sequence (in red). Gathering of Cas9n and gRNAs induces a double stranded break (DSB) in the target region. Absence of a HDR donor template activates NHEJ repair to join the DSB. The “N” and “X” indicated in the NHEJ repaired target sequences denotes insertion or deletion of a few base pairs during DSB repair. Indels formed during the NHEJ repair process result in a frame shift mutation that results in the formation of a non-functional SigI protein. This in return may permanently halt the growth and virulence of the B. anthracis bacterium as SigI is required for the growth of B. anthracis and transcription of toxin genes expression. Thus targeting the SigI gene in the B. anthracis genome and inhibition of its expression by a CRISPR/Cas9-mediated gene editing strategy may suppress the growth and virulence of B. anthracis.

FIG. 15 is a schematic diagram illustrating knock-out of a gene required for survival of a pathogenic virus that affects humans. Complete eradication of Hepatitis B virus (HBV) is most likely impossible without therapies that effectively target the integrated proviral DNA and stable viral covalently closed circular (ccc) DNA molecules present in infected hepatocytes. (A): HBV endocytoses into cells using pre-s-polypeptide and uncoats its nuclear-capsid core before entering into the nucleus. The HBV viral genome is self-repaired by viral polymerase and stores its genetic material as cccDNA. The cccDNA acts as the template to transcribe viral mRNA that will be used for pre-genomic mRNA (pgRNA) and synthesis of viral proteins. The virion core proteins encapsulate the pgRNA and are reverse transcribed by viral reverse transcriptase to synthesize negative strand DNA that is then converted to partially double stranded relaxed circular DNA. The immature HBV then enters into the endoplasmic reticulum where it matures prior to release into the extracellular environment. Targeting the cccDNA and integrated proviral genome using the described CRISPR/Cas9 system can inactivate HBV viral replication in chronically infected patients (indicated in the arrows with dashed lines). Administration of an HBV knockout plasmid that drives synthesis of Cas9n and HBV specific gRNAs leads to cleavage of specific sequences and creates a DSB. Repair of this DSB by the cellular NHEJ machinery can introduce indels that often impair viral DNA function. (B): The diagram shows the reverse transcriptase (RT) gene sequence of the HBV viral genome. Cas9n recruitment to the RT gene is mediated by dual gRNAs that contain a 20 bp protospacer recognizing sequence (in green) followed by a PAM sequence (in red). Gathering of Cas9n and gRNAs induces a double stranded break (DSB) in the target region. Absence of HDR donor template activates NHEJ repair to join the DSB. The “N” and “X” indicated in the NHEJ repaired target sequences denotes insertion or deletion of a few base pairs during DSB repair. Indels formed during the NHEJ repair process may permanently inhibit RT (pol) gene expression by frame shift mutations in both cccDNA and integrated HBV sequence.

FIG. 16 is a schematic diagram illustrating knock-out of a gene required for survival of a pathogenic yeast that affect humans. The diagram shows the calcineurin gene sequence of the Candida albicans genome (causing candidiasis). Cas9n recruitment to the calcineurin gene is mediated by dual gRNAs that contain a 20 bp protospacer recognizing sequence (in green) followed by a PAM sequence (in red). Gathering of Cas9n and gRNAs induces a DSB in the target region. Absence of HDR donor template activates NHEJ repair to join the DSB. The “N” and “X” indicated in the NHEJ repaired target sequences denotes insertion or deletion of a few base pairs during DSB repair. Indels formed during the NHEJ repair process result in a frame shift mutation that results in the formation of a non-functional calcineurin protein. This in return may permanently halt the growth and virulence of the Candida albicans as Calcineurin is required for the virulence of C. albicans.

FIG. 17 is a schematic diagram illustrating knock-out of a gene that results in a prion disease such as Creutzfeldt-Jakob Disease. The diagram shows a prion protein gene sequence in the human genome. Cas9n recruitment to the prion protein gene is mediated by dual gRNAs that contain a 20 bp protospacer recognizing sequence (in green) followed by a PAM sequence (in red). Gathering of Cas9n and gRNAs induces a DSB in the target region. Absence of HDR donor template activates NHEJ repair to join the DSB. The “N” and “X” indicated in the NHEJ repaired target sequences denotes insertion or deletion of a few base pairs during DSB repair. Indels formed during the NHEJ repair process can permanently inhibit prion protein gene expression by frame shift mutation. Prion disease involves a conformational transition of α-helix into β-sheet in the prion protein to form the pathogenic prion protein which then interacts with normal cellular prion protein and converts it into the pathogenic form. Methods described here may thus suppress the conversion of the normal prion protein into the pathogenic form and slow down or eradicate the aggregation of pathogenic prions in the brain.

FIG. 18 is a schematic diagram illustrating knock-out of a previously inserted transgene in the germ line of a genetically engineered animal (in this example, a GFP-expressing transgenic fish). The diagram shows the green fluorescent protein (GFP) gene sequence from Aequorea victoria that has been previously inserted into the fish genome. Cas9n recruitment to the GFP gene is mediated by dual gRNAs that contain a 20 bp protospacer recognizing sequence (in green) followed by a PAM sequence (in red). Gathering of Cas9n and gRNAs induces a DSB in the target region. Absence of HDR donor template activates NHEJ repair to join the DSB. The “N” and “X” indicated in the NHEJ repaired target sequences denotes insertion or deletion of a few base pairs during DSB repair. Indels formed during the NHEJ repair process results in a frame shift mutation that results in the formation of a non-functional GFP protein. Thus methods described herein may be used to knock-out a previously inserted transgene in the germ line of a genetically engineered animal.

FIG. 19 is a schematic diagram illustrating knock-out of a previously inserted transgene in the germ line of a genetically engineered plant (in this example, an α-interferon transgene-expressing genetically-engineered plant). The diagram shows the human α-interferon gene sequence that has been previously inserted into a genetically-engineered plant. Cas9n recruitment to the α-interferon gene is mediated by dual gRNAs that contain a 20 bp protospacer recognizing sequence (in green) followed by a PAM sequence (in red). Gathering of Cas9n and gRNAs induces a DSB in the target region. Absence of HDR donor template activates NHEJ repair to join the DSB. The “N” and “X” indicated in the NHEJ repaired target sequences denotes insertion or deletion of a few base pairs during DSB repair. Indels formed during the NHEJ repair process result in a frame shift mutation that results in the formation of a non-functional α-interferon protein. Thus methods described herein may be used to knock out a previously inserted transgene in the germ line of a genetically engineered plant.

FIG. 20 is a schematic diagram illustrating repeated correction of non-functional genes to increase the incidence of gene correction by HDR. (A): HDR mediated gene-editing strategy that requires multiple transfections of the HDR-plasmid and corrective HDR donor, targeting the disease causing mutation, in this case cystic fibrosis caused by a W1282X mutation, an example of a non-sense or premature termination mutation. Several rounds of HDR-plasmid and corrective HDR template targeting the W1282X mutation to introduce the tryptophan (W) codon to correct the premature termination codon (PTC), increases the number of corrected cells in each round since the design of the ssODN and gRNA prevent further changes in already corrected cells. During this process, several indels may occur in some cells along with HDR-mediated repair and gene correction in other cells, while some cells will not receive the HDR-plasmid and/or corrective HDR donor due to less than 100% efficiency of introduction into all cells at each round. Since the pre-existing PTC already results in completely non-functional protein synthesis, there is no significant safety concern if there is an indel mutation, as the gene is already completely non-functional. Thus every attempt to introduce the HDR-plasmid and corrective HDR template to the cell population (in vitro or in vivo) significantly increases the number of corrected cells, without further harming the remaining cells. This method thus allows for restoration of protein expression efficiently and safely via HDR-mediated genome editing. The same methodology may be applied for treating similar diseases that are caused by non-functional protein synthesis. (B): Design of two gRNAs that exclusively bind to the W1282X mutated region of the cystic fibrosis trans-membrane conductance regulator (CFTR) gene to correct the premature termination codon (PTC) and restore CFTR gene function. (C): Introduction of an ssODN HDR donor template, either sense or anti-sense, induces HDR cellular machinery to join and repair the DSB. The PAM sequence and the 3′ end of the gRNA sequences are masked without changing the amino acid codon to avoid unwanted activity by the CRISPR/Cas9 system in the already corrected CFTR gene sequence.

FIG. 21 is a schematic diagram showing various gRNA sequences designed to target a variety of genes causing genetic disorders or pathogenic diseases or present in genetically-engineered organisms. Dual gRNAs that contain a 20 bp protospacer recognizing sequence (in green) followed by a PAM sequence (in red) are shown. (A): gRNAs for targeting mutated Factor VIII gene; (B): gRNAs for targeting mutated Factor IX gene; (C): gRNAs for targeting mutated HBB gene; (D): gRNAs for targeting mutated IL2RG gene; (E): gRNAs for targeting mutated PD-1 gene; (F): gRNAs for targeting mutated CEP290 gene; (G): gRNAs for targeting mutated RPGR gene; (H): gRNAs for targeting mutated PCSK9 promoter region; (I): gRNAs for targeting beta-hexosaminidase A gene; (J): gRNAs for targeting HBV cccDNA; (K): gRNAs for targeting HCV viral sequence; (L): gRNAs for targeting mutated dystrophin gene; (M): gRNAs for targeting mutated human beta-globin gene; (N): gRNAs for targeting mutated FGFR3 gene; (O): gRNAs for targeting mutated FMR1 gene; (P): gRNAs for targeting NOP56 gene; (Q): gRNAs for targeting mutated POLR1C gene; (R): gRNAs for targeting HSV viral sequence; (S): gRNAs for targeting Plasmodium falciparum; (T): gRNAs for targeting Bacillus anthracis; (U): gRNAs for targeting HBV viral sequence; (V): gRNAs for targeting calcineurin gene in Candidas; (W): gRNAs for targeting human prion gene; (X): gRNAs for targeting cattle prion gene; (Y): gRNAs for targeting GFP-expressing transgenic fish; (Z): gRNAs for targeting alpha-interferon expressing transgenic rice crop.

FIG. 22 is a schematic drawing illustrating knockout of oncogenes that disrupt the normal cell cycle and lead to cancer development. WNT10A gene knockout in Caki-1 cells is provided as an example to demonstrate knockout of oncogenes. (A): The diagram illustrates the exon-2 sequence targeted in the WNT10A gene. Cas9n recruitment to the WNT10A exon-2 gene is mediated by dual gRNAs that contain a 20 bp protospacer recognizing sequence (in green) followed by a PAM sequence (in red). The Cas9n and gRNAs induce a double stranded break (DSB) in the target region. The absence of a HDR donor template activates NHEJ repair to join the DSB in the target sequence. The “N” and “X” indicated in the NHEJ repaired target sequences denotes insertion or deletion of a few base pairs during DSB repair. Indels formed during the NHEJ repair process result in a frame shift mutation in the WNT10A gene that leads to the formation of a non-functional WNT10A protein that inhibits the carcinogenesis and its disease progression. (B, C): Analysis of CRISPR/Cas9 mediated cleavage efficiency using T7 endonuclease I mutation detection assay. An episomal plasmid coding for the gRNAs (pD-EpiWe2gRNA1; SEQ ID NO: 107; shown schematically in FIG. 24) was transfected in Caski-1 cells followed by transfection with Cas9n mRNA. The percentage of edited cells (% indel) in Lane-5 resulting from the indel was estimated using a T7 endonuclease I mutation detection assay by quantitating the amount of cut vs. uncut DNA fragments to be 20% (with no cell selection used anywhere in the process). The samples were run on a 2% agarose gel. Optionally, Cas9n mRNA could also be used to generate two sets of DSBs and even more efficient inactivation of the WNT10A protein.

FIG. 23 illustrates truncated CD4 expression in KG1 cells transfected with a plasmid coding for the gRNA sequences and the truncated CD4 (pD-2CCR5gRNA; SEQ ID NO: 108; shown schematically in FIG. 24) and Cas9n mRNA. (A): Amnis® FlowSight expression analysis of truncated CD4 following transfection with pD-2CCR5gRNA and Cas9n mRNA at different time-points (24 hour, 42 hour and 72 hour post-transfection) in the CD-ve KG1 cells is shown. The peak % cell population of the gated CD4+ stained cells was found to be at ˜48% around 42 h post-transfection.

FIG. 24 shows schematic plasmid maps for various gRNA expression vectors targeting different gene targets, as follows: (A): pD-Epi723gRNA1, a triple gRNA expressing plasmid designed for excision of an extra GGGGCC hexanucleotide repeat in C9ORF72 gene of ALS patient gDNA; (B): pD-EpiWe2gRNA1, a plasmid designed for knockout of WNT10A gene in Caki-1 cells. Both of these plasmids contain a non-integrating episomal sequence for replication of the transfected plasmids to express gRNAs over a sufficient time in the cells, and EGFP protein expression for evaluating transfection efficiency. (C): pD-2CCR5gRNA, a plasmid designed for knockout of CCR5 gene in KG-1 cells. The pD-2CCR5gRNA plasmid contains a truncated CCR5 gene for antibiotic-free selection of transfected cells using magnetic microbeads.

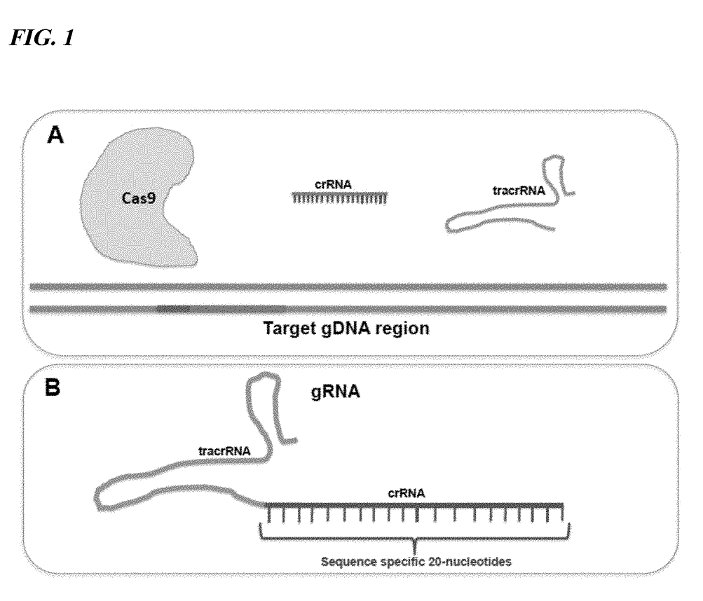

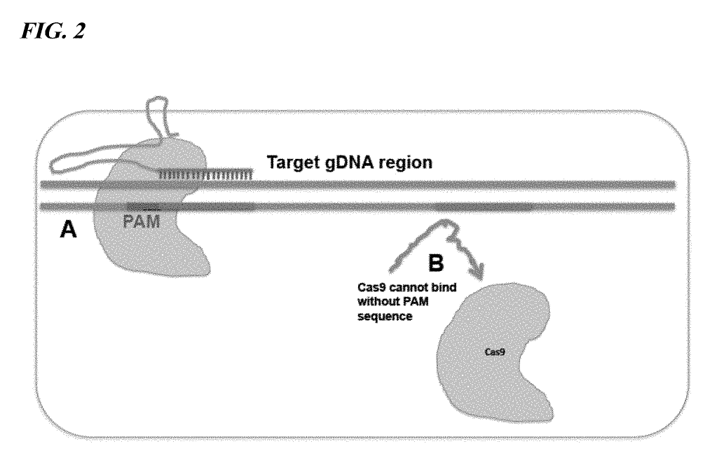

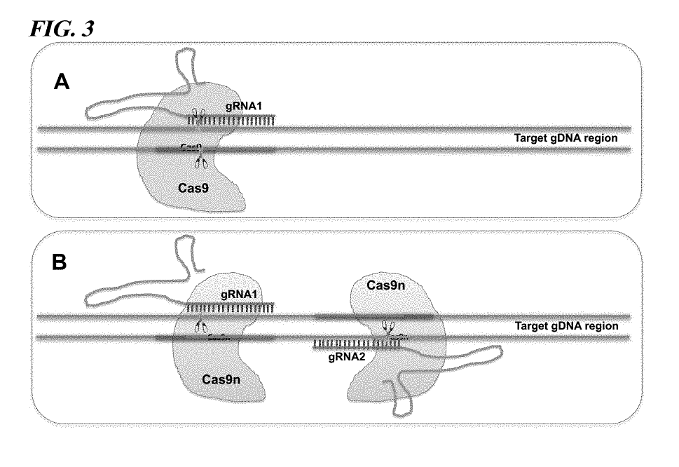

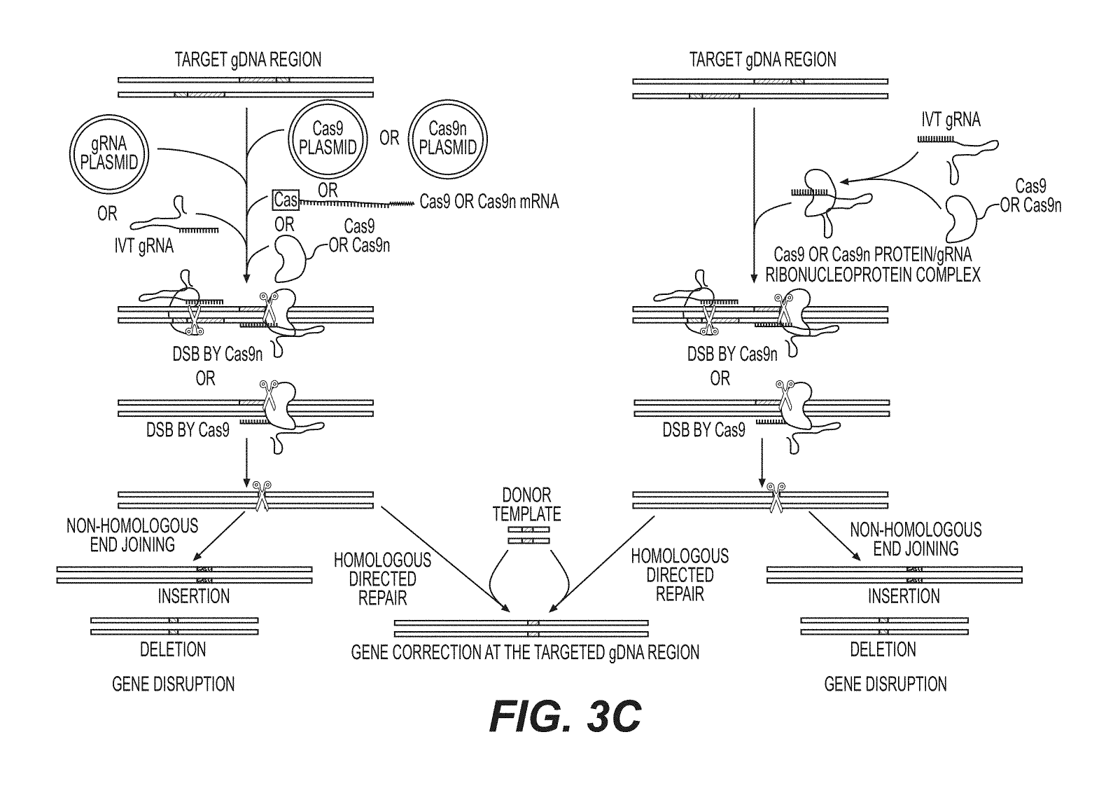

### DETAILED DESCRIPTION

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The terms “derivative” and “variant” are used interchangeably herein.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 200, and more generally of less than about 1000, nucleotides of single- or double-stranded DNA, although there is no upper limit to the length of an oligonucleotide. Oligonucleotides are also referred to as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

“Genomic DNA” refers to the DNA of a genome of an organism including, but not limited to, the DNA of the genome of a bacterium, fungus, archea, plant or animal, including mammals, including humans.

“Manipulating” DNA encompasses binding, nicking one strand, or cleaving (i.e., cutting) both strands of the DNA, or encompasses modifying the DNA or a polypeptide associated with the DNA. Manipulating DNA can (but does not necessarily) silence, activate, or modulate (either increase or decrease) the expression of an RNA or polypeptide encoded by the DNA. Manipulating DNA can (but does not necessarily) alter the amino acid sequence of a polypeptide encoded by the DNA. Such alteration in the amino acid sequence may affect (e.g., increase or decrease) the function, enzymatic activity, and/or stability of the encoded polypeptide.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e. not include any mismatches.

By “hybridizable” or “complementary” or “substantially complementary” it is meant that a nucleic acid (e.g., RNA) comprises a sequence of nucleotides that enables it to non-covalently bind, i.e., to form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) (DNA, RNA). In addition, it is known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position in a protein-binding segment (dsRNA duplex) of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). It is well-known that the conditions of temperature and ionic strength determine the “stringency” of the hybridization.

Hybridization requires that two nucleic acids contain complementary sequences, although mismatches between bases are possible. The conditions appropriate for hybridization between two nucleic acids depend on the length of the nucleic acids and the degree of complementarity, variables well-known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides. Furthermore, the skilled artisan will recognize that the temperature and wash solution salt concentration may be adjusted as necessary according to factors such as length of the region of complementarity and the degree of complementarity.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within a target nucleic acid sequence to which it is targeted. For example, an antisense nucleic acid in which 18 of 20 nucleotides of the antisense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}. By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.), and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/,ebi.ac.uk/Tools/msa/muscle/,mafft.cbrc.ip/aliqnment/software/. See, e.g., Altschul et al. (1990), J. Mol. Bioi. 215:403-10.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, the T7 promoter, etc., may be used to drive the various vectors of the present invention.

A promoter can be a constitutively active promoter (i.e., a promoter that is constitutively in an active/“ON” state), an inducible promoter (i.e., a promoter whose state, active/“ON” or inactive/“OFF”, is controlled by an external stimulus, e.g., the presence of a particular temperature, compound, or protein), a spatially restricted promoter (i.e., transcriptional control element, enhancer, etc.; e.g., a tissue specific promoter, a cell type specific promoter, etc.), and/or a temporally restricted promoter (i.e., the promoter is in the “ON” state or “OFF” state during specific stages of embryonic development or during specific stages of a biological process. Suitable promoters can be derived from viruses and can therefore be referred to as viral promoters, or they can be derived from any organism, including prokaryotic or eukaryotic organisms. Suitable promoters can be used to drive expression by any RNA polymerase (e.g., Pol I, Pol II, or Pol III). Exemplary promoters include, but are not limited to, the SV40 early promoter; mouse mammary tumor virus long terminal repeat (LTR) promoter; adenovirus major late promoter (Ad MLP); a herpes simplex virus (HSV) promoter; a cytomegalovirus (CMV) promoter such as the CMV immediate early promoter region (CMVIE); a rous sarcoma virus (RSV) promoter; a human U6 small nuclear promoter (U6; Miyagishi et al., Nature Biotechnology 20: 497-500, 2002), an enhanced U6 promoter (e.g., Xia et al., Nucleic Acids Res. 31(17), 2003), a human H1 promoter (H1), and the like.

Examples of inducible promoters include, but are not limited to, T7 RNA polymerase promoter, T3 RNA polymerase promoter, Isopropyl-beta-D-thiogalactopyranoside (IPTG)-regulated promoter, lactose-induced promoter, heat shock promoter, tetracycline-regulated promoter, steroid-regulated promoter, metal-regulated promoter, estrogen receptor-regulated promoter, and the like. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on several factors such as the organism. For example, various spatially restricted promoters are known for plants, flies, worms, mammals, mice, etc. Thus, a spatially restricted promoter can be used to regulate the expression of a nucleic acid encoding a site-directed modifying polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process. Many examples of spatially restricted promoters are known and include, without limitation: neuron-specific promoters, adipocyte-specific promoters, cardiomyocyte-specific promoters, smooth muscle-specific promoters, and photoreceptor-specific promoters.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., a guide RNA) or a coding sequence (e.g., a site-directed modifying polypeptide, or a Cas9 polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms. Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence.

Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

A cell has been “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell.

In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of transformation include, e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, direct microinjection, nanoparticle-mediated nucleic acid delivery, and the like. The choice of method of transformation is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a mammalian germ cell), by virtue of introduction into a suitable eukaryotic host cell of an exogenous nucleic acid.

A “target DNA” as used herein is a DNA polynucleotide that comprises a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” By “site-directed modifying polypeptide” or “RNA-binding site-directed polypeptide” or “RNA-binding site-directed modifying polypeptide” or “site-directed polypeptide” is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence).

By “cleavage” is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a site-directed modifying polypeptide is used for targeted double-stranded DNA cleavage.

“Nuclease” and “endonuclease” are used interchangeably herein to mean an enzyme which possesses endonucleolytic catalytic activity for DNA cleavage.

By “cleavage domain” or “active domain” or “nuclease domain” of a nuclease is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

By “site-directed polypeptide” or “RNA-binding site-directed polypeptide” is meant a polypeptide that binds RNA and is targeted to a specific DNA sequence. A site-directed polypeptide as described herein is targeted to a specific DNA sequence by the RNA molecule to which it is bound. The RNA molecule comprises a sequence that is complementary to a target sequence within the target DNA, thus targeting the bound polypeptide to a specific location within the target DNA (the target sequence).

The RNA molecule that binds to the site-directed modifying polypeptide and targets the polypeptide to a specific location within a target DNA is referred to herein as a “guide RNA” or “guide RNA polynucleotide” (also referred to herein as a “gRNA”). A guide RNA typically comprises two segments, a “DNA-targeting segment” and a “protein-binding segment.” By “segment” is meant a segment, section, or region of a molecule, e.g., a contiguous stretch of nucleotides in an RNA. A segment can also mean a region or section of a complex such that a segment may comprise regions of more than one molecule. For example, in some cases the protein-binding segment (described below) of a guide RNA is one RNA molecule and the protein-binding segment therefore comprises a region of that RNA molecule. In other cases, the protein-binding segment (described below) of a guide RNA comprises two separate molecules that are hybridized along a region of complementarity. As an illustrative, non-limiting example, a protein-binding segment of a guide RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA sequence or target genomic DNA (gDNA) region (the complementary strand of the target DNA) designated the “protospacer-like” sequence herein. The protein-binding segment (or “protein-binding sequence”) interacts with a site-directed modifying polypeptide. When the site-directed modifying polypeptide is a Cas9 or Cas9 related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA sequence or target gDNA.

As used herein, the terms “target genomic DNA (gDNA)”, “target genomic DNA (gDNA) region”, and “gDNA” are used interchangeably to refer to a target DNA sequence present in the genome of a cell, i.e., a chromosomal target DNA sequence. In some cases, where it is clear that the target DNA sequence is present in the genome of a cell, the terms “target DNA sequence” and “target genomic DNA (gDNA) region” and “gDNA” may be used interchangeably.

The protein-binding segment of a guide RNA comprises, in part, two complementary stretches of nucleotides that hybridize to one another to form a double stranded RNA duplex (dsRNA duplex).

In some embodiments, a nucleic acid (e.g., a guide RNA, a nucleic acid comprising a nucleotide sequence encoding a guide RNA; a nucleic acid encoding a site-directed polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some embodiments, a guide RNA comprises an additional segment at either the 5′ or 3′ end that provides for any of the features described above. For example, a suitable third segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guide RNA and a site-directed modifying polypeptide (i.e., site-directed polypeptide) form a complex (i.e., bind via non-covalent interactions). The guide RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence (e.g. a target sequence in a chromosomal nucleic acid, e.g., a genome; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

In some embodiments, a guide RNA comprises two separate RNA molecules (RNA polynucleotides: an “crRNA” and a “tracrRNA”, see below) and may be referred to herein as a “double-molecule guide RNA” or a “two-molecule guide RNA.” In other embodiments, the guide RNA is a single RNA molecule (single RNA polynucleotide) and may be referred to herein as a “single-molecule guide RNA,” a “single-guide RNA,” or an “sgRNA.” The term “guide RNA” or “gRNA” is inclusive, referring both to double-molecule guide RNAs and to single-molecule guide RNAs (i.e., sgRNAs).

An exemplary single-molecule guide RNA comprises a CRISPR RNA (crRNA or crRNA-like) molecule which includes a CRISPR repeat or CRISPR repeat-like sequence and a corresponding trans-activating crRNA (tracrRNA or tracrRNA-like) molecule. A crRNA molecule comprises both the DNA-targeting segment (single stranded) of the guide RNA and a stretch (a duplex-forming segment) of nucleotides that forms one half of the dsRNA duplex of the protein-binding segment of the guide RNA. The corresponding tracrRNA molecule comprises a stretch of nucleotides (a duplex-forming segment) that forms the other half of the dsRNA duplex of the protein-binding segment of the guide RNA. In other words, a stretch of nucleotides of the crRNA molecule are complementary to and hybridize with a stretch of nucleotides of the tracrRNA molecule to form the dsRNA duplex of the protein-binding domain of the guide RNA. As such, each crRNA molecule can be said to have a corresponding tracrRNA molecule. The crRNA molecule additionally provides the single stranded DNA-targeting segment. Thus, a crRNA and a tracrRNA molecule (as a corresponding pair) hybridize to form a guide RNA. A double-molecule guide RNA can comprise any corresponding crRNA and tracrRNA pair.

A single-molecule guide RNA comprises two stretches of nucleotides (a crRNA and a tracrRNA) that are complementary to one another, are covalently linked (directly, or by intervening nucleotides), and hybridize to form the double stranded RNA duplex (dsRNA duplex) of the protein-binding segment, thus resulting in a stem-loop structure. The crRNA and the tracrRNA can be covalently linked via the 3′ end of the crRNA and the 5′ end of the tracrRNA. Alternatively, crRNA and the tracrRNA can be covalently linked via the 5′ end of the crRNA and the 3′ end of the tracrRNA.

The term “stem cell” is used herein to refer to a cell (e.g., a vertebrate stem cell) that has the ability both to self-renew and to generate a differentiated cell type (see Morrison et al., Cell 88:287-298, 1997). In the context of cell ontogeny, the adjective “differentiated”, or “differentiating” is a relative term. A “differentiated cell” is a cell that has progressed further down the developmental pathway than the cell it is being compared with. Thus, pluripotent stem cells can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism.

PSCs of animals can be derived in a number of different ways. For example, embryonic stem cells (ESCs) are derived from the inner cell mass of an embryo (Thomson et. al, Science 282: 5391, 1998) whereas induced pluripotent stem cells (iPSCs) are derived from somatic cells (Takahashi et. al, Cell 131 (5):861-72, 2007; Yu et. al, Science318(5858):1917-20, 2007). Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “embryonic stem cell” (ESC) is meant a PSC that was isolated from an embryo, typically from the inner cell mass of the blastocyst. ESC lines are listed in the NIH Human Embryonic Stem Cell Registry and many such lines are known. Stem cells of interest also include embryonic stem cells from other primates, such as Rhesus stem cells and marmoset stem cells. It should be understood that stem cells may be obtained from any mammalian species, e.g., human, equine, bovine, porcine, canine, feline, rodent, e.g. mice, rats, hamsters, primates, etc. In culture, ESCs typically grow as flat colonies with large nucleo-cytoplasmic ratios, defined borders and prominent nucleoli. Examples of methods of generating and characterizing ESCs may be found in, for example, U.S. Pat. Nos. 7,029,913, 5,843,780, and 6,200,806, the disclosures of which are incorporated herein by reference. Methods for proliferating hESCs in undifferentiated form are described in WO 99/20741, WO 01/51616, and WO 03/020920. By “embryonic germ stem cell” (EGSC) or “embryonic germ cell” or “EG cell” is meant a PSC that is derived from germ cells and/or germ cell progenitors, e.g. primordial germ cells, i.e., those that would become sperm and eggs. Embryonic germ cells (EG cells) are thought to have properties similar to embryonic stem cells as described above.

By “induced pluripotent stem cell” or “iPSC” is meant a PSC that is derived from a cell that is not a PSC (i.e., from a cell that is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs may be found in, for example, U.S. Patent Application Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell” is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components. By “post-mitotic cell” is meant a cell that has exited from mitosis, i.e., is “quiescent”, i.e. no longer undergoing divisions. This quiescent state may be temporary, i.e. reversible, or it may be permanent. Similarly, by “meiotic cell” is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

By “recombination” is meant a process of exchange of genetic information between two polynucleotides. As used herein, “homology-directed repair (HDR)” refers to the specialized form of DNA repair that takes place, for example, during repair of double-strand breaks in cells. This process requires nucleotide sequence homology, uses a “donor” molecule as a template for repair of a “target” molecule (i.e., the one that experienced the double-strand break), and leads to the transfer of genetic information from the donor to the target. The donor is also referred to herein as the “donor template” and the “repair template.” Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target DNA.

By “non-homologous end joining (NHEJ)” is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence(s) near the site of the double-strand break.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it should be understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The invention further provides kits for genomic DNA modification in a mammalian cell. Kits may comprise one or more guide RNA, repair template, and Cas9 protein (or nucleic acid encoding a Cas9 protein), and/or instructions for use. A kit may also include reagents, solvents, buffers, etc., required for carrying out the methods described herein. In some embodiments, a kit includes a ssODN as described herein for use as a repair template and one or more guide RNA, or one or more nucleic acid (such as a vector) encoding the ssODN and/or the guide RNA.

It should be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

We demonstrate herein that the described CRISPR/Cas systems can be used for several gene editing-based therapeutic applications, such as but not limited to, the removal of extra hexanucleotide repeats within the C9ORF72 gene at chromosome 9 and correction of the H46R mutation in SOD1 gene that causes amyotrophic lateral sclerosis (ALS). In addition, we demonstrate that the aforementioned CRISPR/Cas system-based genetic manipulation strategy can also be used for inactivating the expression of CCR5, a co-receptor for CD4, which is required for viral entry into cells and implicated in human immunodeficiency virus (HIV)'s mode of infection. Other potential therapeutic applications are also illustrated.

It is noted that, among the different types of Cas enzymes, type-II Cas9 protein has been used as the CRISPR nuclease for gene editing-based therapeutic strategies described herein, however the particular Cas enzyme used is not meant to be limited. It should be expressly understood that any suitable Cas enzyme may be used in methods and systems provided herein.

### Example 1. Construction of CRISPR Plasmids

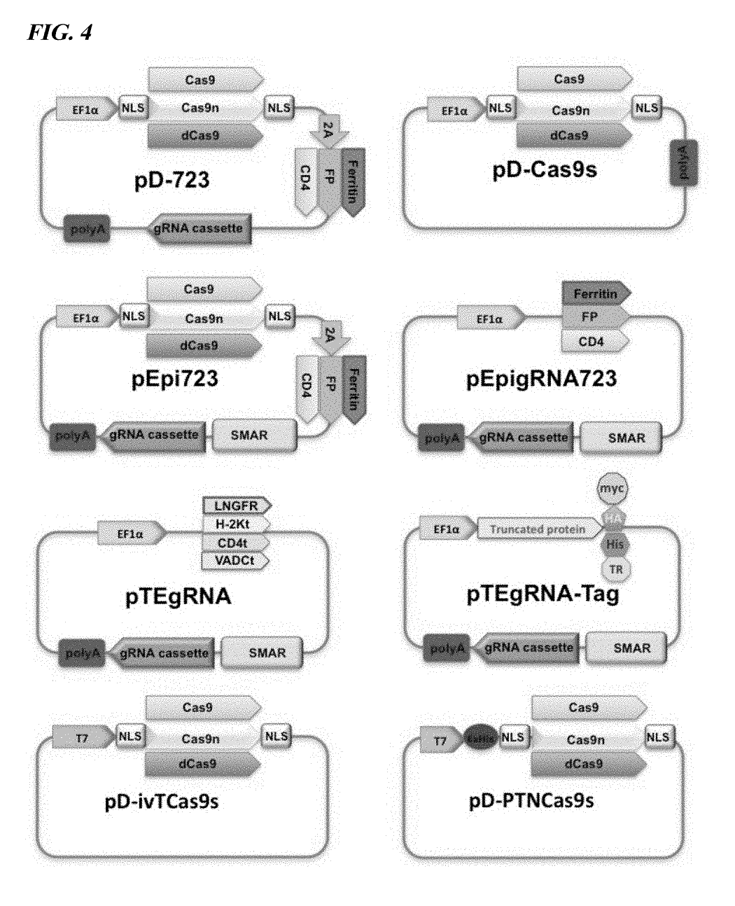

General procedures for construction of CRISPR plasmids for use in the CRISPR/Cas system (e.g., with Cas9 from Streptococcus pyogenes) to make genomic modifications are described below. Genomic modifications that can be made using the CRISPR/Cas system provided herein include, without limitation: induction of a gene mutation; correction of a mutated sequence; deletion or insertion of a sequence; and gene repression or activation. Steps include, without limitation: identification of gRNA target sites; analysis of off-target activities; construction of CRISPR plasmids; and transfection of CRISPR components into cells (e.g., cell lines) of interest. CIRSPR plasmids may be all-in-one plasmids or dual plasmids that express Cas9 proteins and gRNAs separately. CRISPR plasmids may be designed for single or multiplex gRNA expression. Non-limiting examples of such plasmids include an “All-in-one” CRISPR plasmid comprising a Promoter-NLS-Cas9n-NLS-2A-reporter-episomal sequence-gRNA cassette; a Cas9n-expressing plasmid that comprising Promoter-NLS-Cas9n-NLS for co-transfection with gRNA-expressing plasmids comprising a Promoter-reporter-episomal sequence-gRNA cassette; T7 promoter-driven Cas9- and gRNA-expressing plasmids; N-terminal 6×His-tagged T7 promoter-driven Cas9 protein expressing prokaryotic vectors for synthesis of recombinant Cas9 protein (and similar plasmids for expressing Cas9n and dCas9). Table 3 and FIG. 4 show exemplary CRISPR plasmids used to introduce CRISPR/Cas components into cells for therapeutic gene editing strategies described herein. It is noted that Cas9n is given as an example only. It should be understood that Cas9n can be replaced with Cas9 or dCas9 as required. Similarly, the fluorescent protein “FP#” is provided as an example of a reporter gene/protein. However, it should be expressly understood that any other suitable reporter gene/protein can be used. Non-limiting examples of such reporter genes/proteins include other fluorescent proteins, CD4, ferritin and the like. Any reporter gene/protein suitable for the isolation or tracking of transgene-expressing cells may be used.

TABLE 3
Exemplary CRISPR plasmids used in CRISPR/Cas systems described herein.

| | Plasmids | Description | Components |
|---|---|---|---|
| 1. | pDN-723 | EF1α promoter driven all-in-one | EF1α-NLS-Cas9n*-NLS-2A-FP^{#}- |
| | | CRISPR plasmid | gRNA_Cassette |
| 2. | pDN-Cas9n | EF1α promoter driven Cas9n | EF1α-NLS-Cas9n*-NLS-hGH_poly(A) |
| | | plasmid | |
| 3. | pEpi-723 | EF1α promoter driven all-in-one | EF1α-NLS-Cas9n*-NLS-2A-FP^{#}- |
| | | CRISPR episomal plasmid | SMAR-gRNA_Cassette |
| 4. | pEpi-gRNA723 | EF1α promoter driven gRNA | EF1α-FP^{#}-SMAR-gRNA_Cassette |
| | | episomal plasmid | |
| 5. | pD-ivTCas9n | T7 promoter driven Cas9n plasmid | T7-NLS-Cas9n*-NLS- |
| | | for ivT mRNA synthesis | |
| 6. | pD-PTNCas9n | T7 promoter driven Cas9n plasmid | T7-HIS6-NLS-Cas9n*-NLS |
| | | for rProtein synthesis | |
| 7. | pDN-gRNA723 | Human U6 and H1 promoters driven | H1-chimeric gRNA-U6-chimeric gRNA |
| | | gRNA plasmid for ivT gRNA | |
| | | synthesis | |

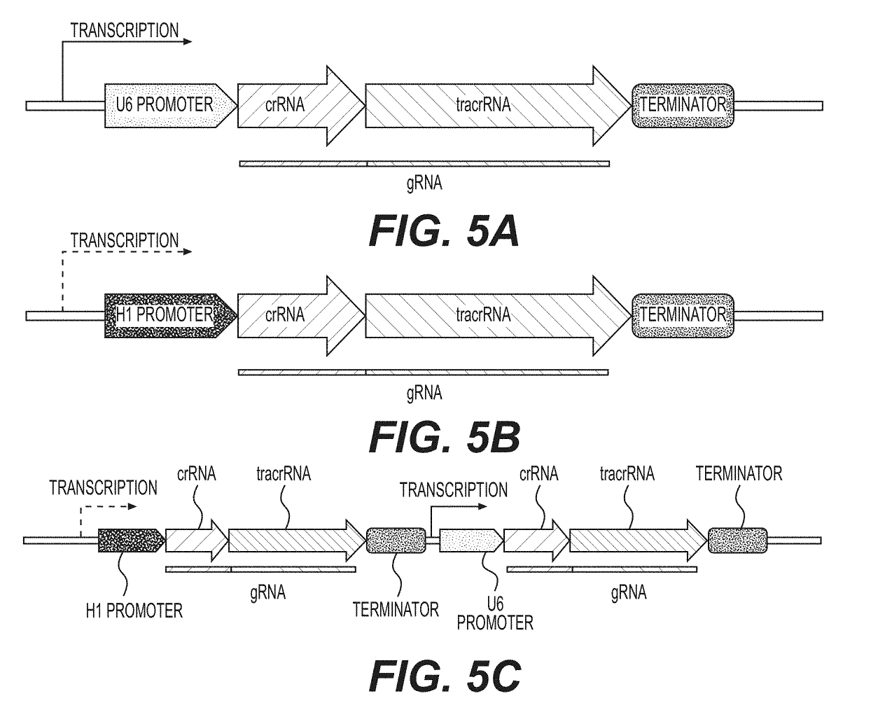

For gRNA expression plasmids, the human U6 and H1 promoters can be used to directly drive transcription to produce gRNA with defined start and end points. Single gRNA expression plasmids constructed with either a human U6 promoter or a human H1 promoter and multiplex gRNA expression plasmids using both human U6 and H1 promoters are produced (FIG. 5). The specificity and accuracy of the CRISPR/Cas system towards the target sequence is determined by how specific the designed gRNA sequence is for the target sequence and the rest of the genomic sequence. Ideally, the gRNA sequence possesses perfect homology to the target region without any homology elsewhere in the genome. Specificity must therefore be considered when designing a gRNA. If the gRNA possesses any homology towards other sequences in the genome, this can lead to off-target effects, which will reduce efficiency and are a major concern for clinical applications.

The following considerations are utilized in methods and systems described herein: i) For Cas9, Cas9n, dCas9, and reporters expression, the human EF1a promoter and human growth hormone poly-A signal are used; ii) Cas9 proteins are tagged with a nuclear localization sequence/signal (NLS) for import into the cell nucleus via nuclear transport; iii) A small self-cleaving 2A peptide sequence is used to construct plasmids expressing multiple proteins from a single mRNA/open reading frame (ORF) (e.g., Cas9 proteins and reporters in the same mRNA); and iv) In addition to conventional plasmids, scaffold/matrix attachment regions (S/MAR) are used for authentic and efficient extra chromosomal (plasmid) replication in mammalian cells to stably express the transgene proteins without integration.

We now elaborate further on the methodology to construct the CRISPR plasmids and preparation of other CRISPR/Cas components for successful genome modification.

A. Single gRNA-Expressing Plasmid Construction Protocol.

First, a target sequence is selected. We design sense and anti-sense DNA oligonucleotide sequences towards the target DNA and upstream of the PAM sequence (5′-NGG-3′). “N” in the PAM sequence stands for any nucleotide (A, C, G, or T). The typical length of the target sequence is 20 bp (e.g., 5′-NNNNNNNNNNNNNNNNNNNNNGG-3′), although in some embodiments shorter or longer target sequences may be used. The PAM sequence (5′-NGG-3′) is shown in bold and underlined.

Next, gRNA oligonucleotides are designed. Two 5′-phosphorylated DNA oligonucleotides are designed, as shown:

5′-ATCCNNNNNNNNNNNNNNNNNNNN-3′
3′-NNNNNNNNNNNNNNNNNNNNCAAA-5′

Next, the two phosphorylated DNA oligonucleotides are annealed together. Primers are diluted to 10 μM using Nuclease buffer or NTE buffer. NTE buffer contains 50 mM NaCl, 10 mM Tris pH7.4, and 1 mM EDTA. The annealing reaction is prepared to generate a duplex as follows: 10 μL of 10 μM top strand oligo and 10 μL of 10 uM bottom strand oligo are mixed together in a total volume of 20 μL and incubated at 95° C. for 5 minutes. After the 5 min. incubation, Oligos were then cooled down from 95° C. to room temperature at the rate of 1° C./min.

Next, the oligo duplex is ligated into the CRISPR vector. Annealed oligos are cloned into a CRISPR plasmid (e.g., an All-in-one CRISPR plasmid) as follows: 1.0 μL linearized All-in-one CRISPR vector, 3.0 μL annealed oligo mix, 1.0 μL 5× Ligation buffer, and 0.25 μL T4 quick ligase are mixed together in a total volume of 5.0 μL and incubated under standard conditions. The mix is then transformed into competent cells with appropriate antibiotic selection using standard methods. The constructed plasmids are confirmed using restriction analysis and DNA sequencing. The CRISPR/Cas system is transfected using a standard transfection protocol, e.g., by lipofection (such as with Lipofectamine LTX™ (Invitrogen)) or by electroporation (such as with 4D Nucleofector™ system (Lonza)), and a functional assay using the SURVEYOR™ mutation detection kit (#706020, IDT) is performed to validate the CRISPR-mediated genome editing in the target gDNA sequence. This assay uses enzymes that cleave heteroduplex DNA of an edited sequence and provides specific information on the mutation's location, orientation, and type.

Dual gRNA Expressing-Plasmid Construction.

First, a dual gRNA expression fragment is synthesized. Forward and reverse primers for generating the desired dual gRNA PCR amplicon are designed and made. Primers may also be procured from a commercial source, e.g., from Sigma Genosys. After the correct size of the amplicon is generated and gel-purified, it is then inserted into a suitable linearized vector (e.g., the All-in-one CRISPR vector).

Primers are designed as shown below:

Forward Primer:

5′-AGACACCTTGGATCCNNNNNNNNNNNNNNNNNNNNGTTTTAGAGCTAGA AATAGCAAG-3′, where N denotes the gRNA1 sequence.

Reverse Primer:

5′-TTCTAGCTCTAAAACnnnnnnnnnnnnnnnnnnnnGTTTTAGAGCTAGAAATAGCA AG-3′, where n denotes the reverse complement sequence of gRNA2.

U6 gBlock is used for this amplicon generation, as follows: 15.0 μL PCR Master mix (2×), 3.0 μL 10 μM forward primer, 3.0 μL 10 μM reverse primer, 3.0 μL DMSO, 0.5 μL gBlock with human U6 promoter, and 5.5 μL Nuclease Free Water are mixed together in a total volume of 30.0 μL. A PCR reaction program is then run as shown in Table 4.

TABLE 4
PCR reaction program.
PCR Program

| Step | Temperature | Duration | |
|---|---|---|---|
| Initial Denaturation | 98° C. | 3 | minutes |
| Amplification | 98° C. | 10 | Seconds |
| (30 Cycles) | 51° C. | 10 | Seconds |
| | 72° C. | 30 | Seconds |
| Final Extension | 72° C. | 10 | minutes |

| | | |
|---|---|---|
| Hold | 4° C. | ∞ |

After completion of PCR amplification, the PCR product (15 μl/well) is resolved by 1.5% agarose gel electrophoresis. If no additional bands are observed, the single PCR amplicon is excised out from the gel and eluted out using column purification.

The PCR product is then cloned into the appropriate vector. For example, the PCR product may be cloned into a linearized All-in-one CRISPR vector as follows: 1.0 μL linearized All-in-one CRISPR vector, 1.0 μL PCR insert (up to 200 ng), 6.0 μL Nuclease free water, and 2.0 μL 5× fusion master mix are mixed together in a total volume of 10 μL. Volumes of the PCR insert and the nuclease free water are varied depending on the concentration of the insert. The linearized vector and PCR insert are generally mixed in a 1:2 molar ratio. The mixture is transformed into competent cells with appropriate antibiotic selection. The constructed plasmids are confirmed by restriction analysis and DNA sequencing. The CRISPR/Cas system is then transfected into mammalian cells using a standard transfection protocol, e.g., by lipofection (such as with Lipofectamine LTX™ (Invitrogen)) or by electroporation (such as with 4D Nucleofector™ system (Lonza)), and a functional assay using the SURVEYOR™ mutation detection kit (#706020, IDT) is performed to validate the CRISPR-mediated genome editing in the target gDNA sequence. This assay uses enzymes that cleave heteroduplex DNA of an edited sequence and provides specific information on the mutation's location, orientation, and type.

Triple gRNA Expressing Plasmid Construction.

First, a triple gRNA expression fragment is synthesized. Forward and reverse primers for generating the desired triple gRNA PCR amplicon are designed and synthesized or procured from, e.g., Sigma Genosys. After the correct sizes of the amplicon are generated and gel purified, the purified amplicon is inserted using a fusion reaction with a suitable linearized vector, such as the All-in-one CRISPR vector.

Primers are designed as shown below:

Amplicon-1 Forward Primer:

5′-AGACACCTTGGATCCNNNNNNNNNNNNNNNNNNNNGTTTTAGAGCTAGA AATAGCAAG-3′, where “N” denotes gRNA1 sequence.

Amplicon-1 Reverse Primer:

5′-nnnnnnnnnnnnnnnnnnnnCGGTGTTTCGTCCTTTCCAC-3′, where “n” denotes reverse complement sequence of gRNA2.

U6 gBlock is used for this amplicon generation.

Amplicon-2 Forward Primer:

5′-NNNNNNNNNNNNNNNGTTTTAGAGCTAGAAATAGCAAG-3′, where “N” denotes gRNA2 sequence.

Amplicon-2 Reverse Primer:

5′-TTCTAGCTCTAAAACnnnnnnnnnnnnnnnnnnnnGGATCCAAGGTGTCTCATAC-3′, where “n” denotes reverse complement sequence of gRNA3.

H1 gBlock is used for this amplicon generation.

For the PCR reaction, components are mixed as follows: 15.0 μL PCR Master mix (2×), 3.0 μL 10 μM forward primer, 3.0 μL 10 μM reverse primer, 3.0 μL DMSO, 0.5 μL gBlock with human U6/H1 promoter, and 5.5 μL Nuclease Free Water are mixed together in a total volume of 30.0 μL. A PCR reaction program is then run as shown in Table 4.

After completion of PCR amplification, the PCR product (15 μL/well) is resolved by 1.5% agarose gel electrophoresis. If no additional bands are observed, the PCR amplicon is excised and eluted out using column purification. The PCR product is then cloned into a vector, such as the All-in-one CRISPR vector, as follows: 1.0 μL linearized All-in-one CRISPR vector, 1.0 μL of each PCR insert in a 1:1 ratio, up to 200 ng, 5.0 μL Nuclease free water, and 2.0 μL 5× fusion master mix are mixed together in a total volume of 10.0 μL. Volumes of the PCR inserts and the nuclease free water are varied depending on the concentration of the inserts. The linearized vector and PCR inserts are generally mixed in a 1:2 molar ratio. The mixture is transformed into competent cells with appropriate antibiotic selection. The constructed plasmids are confirmed by restriction analysis and sequencing. The CRISPR/Cas system is transfected using a standard transfection protocol and a functional assay is performed to validate the CRISPR-mediated genome editing, as described above.

Multiplex gRNA-Expressing Plasmid Construction for Expression of 4 gRNAs in the all-in-One CRISPR Plasmid.

First, a quad (4) gRNA expression fragment is synthesized. Forward and reverse primers for generating the desired quad gRNA PCR amplicon are designed and synthesized or procured from e.g. Sigma Genosys. After the correct sizes of the amplicon are generated and gel purified, the purified amplicon is inserted using a fusion reaction with a suitable linearized vector (such as the All-in-one CRISPR vector). The four gRNA expressing CRISPR plasmid can be used for removal of a defined gene, fragment, or sequence in the genome using Cas9n with significantly high specificity.

Primers are designed as shown:

Amplicon-1 Forward Primer:

5′-AGACACCTTGGATCCNNNNNNNNNNNNNNNNNNNNGTTTTAGAGCTAGA

AATAGCAAG-3′, where “N” denotes gRNA1 sequence.

Amplicon-1 Reverse Primer:

5′-nnnnnnnnnnnnnnnnnnnnCGGTGTTTCGTCCTTTCCAC-3′, where “n” denotes reverse complement sequence of gRNA2.

U6 gBlock is used for this amplicon generation.

Amplicon-2 Forward Primer:

5′-NNNNNNNNNNNNNNNGTTTTAGAGCTAGAAATAGCAAG-3′, where “N” denotes 15 bp of gRNA2 sequence.

Amplicon-2 Reverse Primer:

5′-nnnnnnnnnnnnnnnnnnnnGGATCCAAGGTGTCTCATAC-3′, where “n” denotes reverse complement sequence of gRNA3.

H1 gBlock is used for this amplicon generation.

Amplicon-3 Forward Primer:

5′-NNNNNNNNNNNNNNNGTTTTAGAGCTAGAAATAGCAAG-3′, where “N” denotes 15 bp of gRNA3 sequence.

Amplicon-3 Reverse Primer:

5′-TTCTAGCTCTAAAACnnnnnnnnnnnnnnnnnnnnCGGTGTTTCGTCCTTTCCAC-3′, where “n” denotes reverse complement sequence of gRNA4.

U6 gBlock is used for this amplicon generation.

For the PCR reaction, components are mixed as follows: 15.0 μL PCR Master mix (2×), 3.0 μL 10 μM forward primer, 3.0 μL 10 μM reverse primer, 3.0 μL DMSO, 0.5 μL gBlock with human U6/H1 promoter, and 5.5 μL Nuclease Free Water are mixed together in a total volume of 30.0 μL. A PCR reaction program is then run as shown in Table 4.

After completion of PCR amplification, the PCR product (15 μL/well) is resolved by 1.5% agarose gel electrophoresis. If no additional bands are observed, the PCR amplicon is excised and eluted out using column purification. The PCR product is then cloned into a vector, such as the All-in-one CRISPR vector, as follows: 1.0 μL linearized All-in-one CRISPR vector, 1.0 μL of each PCR insert in a 1:1:1 ratio, up to 200 ng, 4.0 μL Nuclease free water, and 2.0 μL 5× fusion master mix are mixed together in a total volume of 10.0 μL. Volumes of the PCR inserts and the nuclease free water are varied depending on the concentration of the inserts. The linearized vector and PCR inserts are generally mixed in a 1:2 molar ratio. The mixture is transformed into competent cells with appropriate antibiotic selection. The constructed plasmids are confirmed by restriction analysis and sequencing. The CRISPR/Cas system is transfected using a standard transfection protocol and a functional assay is performed to validate the CRISPR-mediated genome editing, as described above.

Preparation of CRISPR Plasmids.

For transfection or microinjection delivery of CRISPR genome editing systems into cells, plasmids must be pure and free from chemical contamination, endotoxins and any animal components. We use an endotoxin-free plasmid DNA maxiprep kit (EndoFree™ Plasmid Maxi Kit, #12362, Qiagen) to isolate plasmid DNA from RecA⁻ and EndA⁻ E. coli cells after overnight culture. Before using the CRISPR plasmids, the isolated plasmids are tested and validated using PCR, restriction analysis and whole plasmid sequencing.

In Vitro Transcription (ivT) for mRNA and gRNA Synthesis.

Plasmids containing T7 promoter-driven gRNA and Cas9 are used in ivT reactions to generate mature Cas9/Cas9n/dCas9 and gRNA. Transient expression of CRISPR components using ivTRNA is integration free and expression decreases as RNA is degraded within the cell.

In vitro transcription of Cas9 protein (such as Cas9, Cas9n, dCas9, etc.) is carried out using INCOGNITO T7 ARCA™ 5 mC- & ψ-RNA transcription kit. The mRNA for the appropriate Cas9 protein is generated in an animal component-free production process using T7 promoter based transcription and subsequent 5′ capping and 3′ polyadenylation. The introduction of anti-reverse cap analog (ARCA) and modified nucleotides (5′-mCTP and ψ-TP) results in higher levels of protein translation and induces a lower innate immune response against the resulting RNAs in downstream applications.

For in vitro transcription of gRNA(s), we introduce a target 20-nucleotide sequence under the control of the T7 promoter that has been amplified by PCR. The PCR amplicon contains T7 promoter+target specific crRNA+tracrRNA construct which is used as a template to synthesis gRNA by T7 RNA polymerase. The resulting products are treated with DNase-I to remove the template and quantified using NanoDrop™. Prior to transfection or microinjection, the activity of the newly synthesized gRNA is checked with Cas9 nuclease and a corresponding template.

CRISPR Nuclease Production.

Plasmids and mRNAs for CRISPR nuclease (Cas9, Cas9n, dCas9, etc.) require transcription and translation for use in genomic modification. Unlike plasmids and mRNAs, Cas9 protein works immediately after transfection into cells with gRNAs.

Another advantage of this Cas9 protein is that we can check the efficiency using in vitro experiments. Humanized Cas9 protein (e.g., Cas9, Cas9n and dCas9) sequences are sub-cloned into a T7-driven E. coli expression vector that contains a nuclear localization signal, an HA epitope, a 6×His tag at the N-terminal, and can be induced with IPTG in BL21 (DE3) strain. Cas9 proteins are purified using Ni-NTA agarose beads, dialyzed and analyzed by SDS-PAGE prior to downstream applications.

Delivery of CRISPR/Cas9 System into Cells.

Methods of delivery are determined based on the target cells and applications desired for genome editing. CRISPR reagents can be delivered by any suitable method such as, without limitation, transfection, nucleofection, and microinjection, and using either plasmid DNA, RNA and/or protein.

### Example 2. Donor Single Stranded DNA Oligonucleotides (ssODN) Design and Optimization

Design of ssODN.

Homology-directed repair (HDR) is a precise genetic modification ranging from a single nucleotide change to large insertions at a pre-defined target site of the genome. However, HDR needs a donor template which could be either a double stranded linear DNA or a ssODN containing the desired sequence. The donor template must be introduced into the cells with the Cas9 or Cas9n enzyme and the gRNA(s). Along with the desired modifications, the donor template must contain an additional homologous sequence immediately downstream and upstream (i.e., a homology sequence at both the right and left arms) of the target sequence.

For large modifications (>100 bp insertion/deletion), using a double stranded DNA donor is generally more efficient than a ssODN. However, a ssODN provides a more effective HDR than a dsDNA template when small modifications (<50 bp) in the target sequence are needed. However, the orientation and length of the ssODN (desired modifications in the offset plus homology arms on each side) must be optimized for each target in order to achieve a high-performance ssODN. We have observed that ssODNs longer than an optimum length result in decreased HDR efficiency (data not shown).

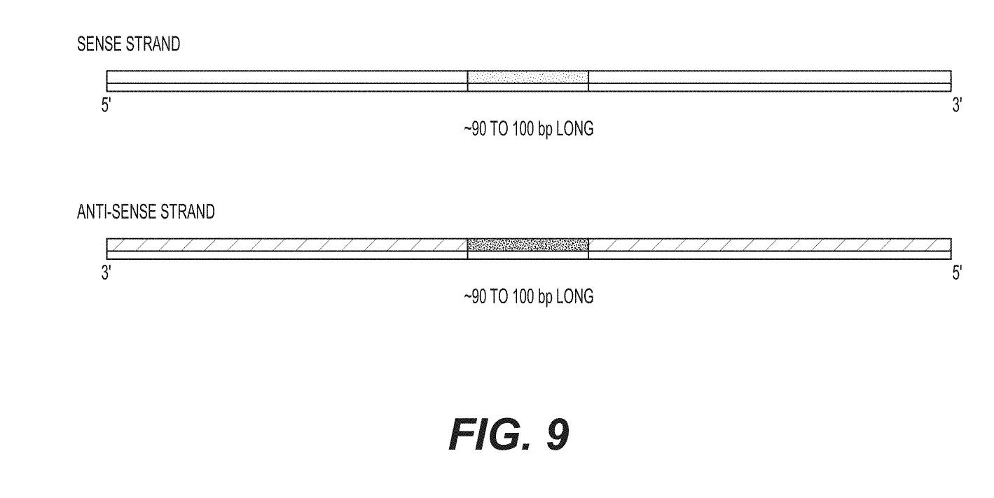

A schematic diagram of the structure of ssODN donor templates is shown in FIG. 9.

Enhancing HDR Efficiency Using Modifications in ssODN and Inhibiting NHEJ.

Compared to NHEJ, HDR occurs at a much lower frequency and is therefore less efficient than NHEJ. This low efficiency of HDR presents a major constraint in the execution of precise genetic modifications by the CRISPR/Cas9 system. In addition to optimal HDR template design, we show that certain modifications can be made in the donor template to improve the stability of the ssODN HDR donor template.

For successful genome modification by Cas9 nuclease, a PAM sequence at the 3′ end of the 20-nucleotide target sequence is required. However, if the HDR template has an intact PAM sequence or retains an intact PAM sequence in the donor template after Cas9 modification has occurred, then it may be degraded by Cas9 in the cells and Cas9 may repeatedly act on the target sequence, even after the desired modification has been introduced. To avoid these unwanted activities by the CRISPR/Cas9 system, we mask the PAM sequence in the HDR donor template by mutating the PAM sequence. For example, in the case of the SpCas9 enzyme, the PAM sequence “NGG” in the HDR template can be mutated to NGT, NGC or NGA. It is noted that, if the HDR template falls within the coding region, then a silent mutation strategy should be followed to avoid introducing amino acid changes into the coding region.

### Example 3. Purification of CRISPR/Cas9 System Edited Cells

Once a large cell population is successfully obtained with a reasonable indel rate or HDR-mediated correction or HDR-mediated knock-in, edited cells will be purified using magnetic separation or other suitable methods known in the art such as, e.g., cell sorting. In the case of magnetic separation, the corrected or edited cells have a change in expression of a cell surface marker or intracellular marker that can be specifically recognized by an antibody (or other means) to which a magnetic core (such as iron nanoparticle microbeads) is attached, allowing for magnetic separation using a magnetic field. Alternatively, the non-corrected/non-edited cells express the cell surface marker or intracellular marker that can be specifically recognized by an antibody (or other means) to which a magnetic core (such as iron nanoparticle microbeads) is attached, allowing for magnetic separation from the corrected or edited cells using a magnetic field (e.g., using a column placed in a magnetic field). In this case, the CRISPR-edited cells are purified by either negative selection (e.g., in the case of a knockout) or positive selection (e.g., in the case of knock-in/HDR repair).

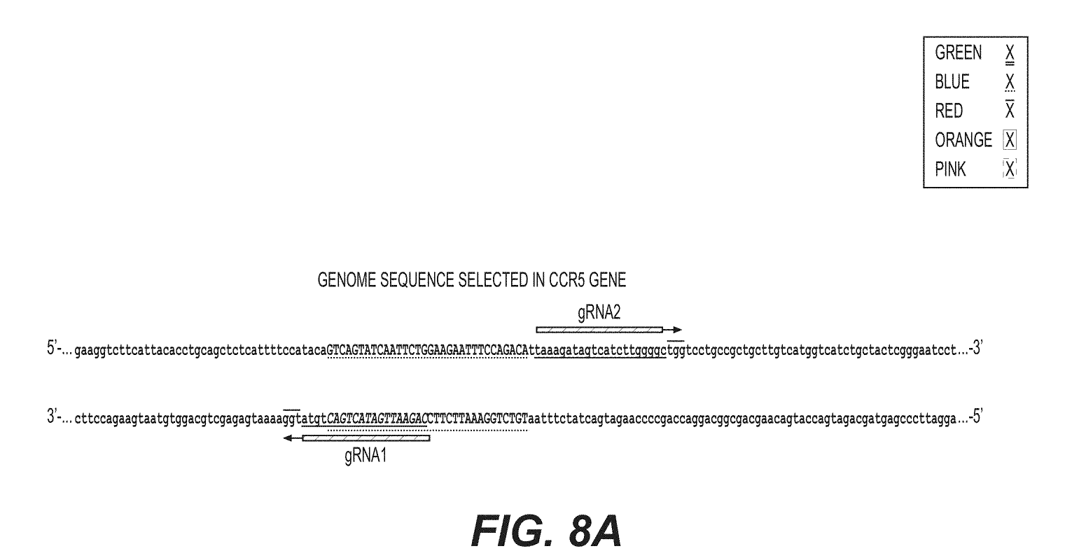

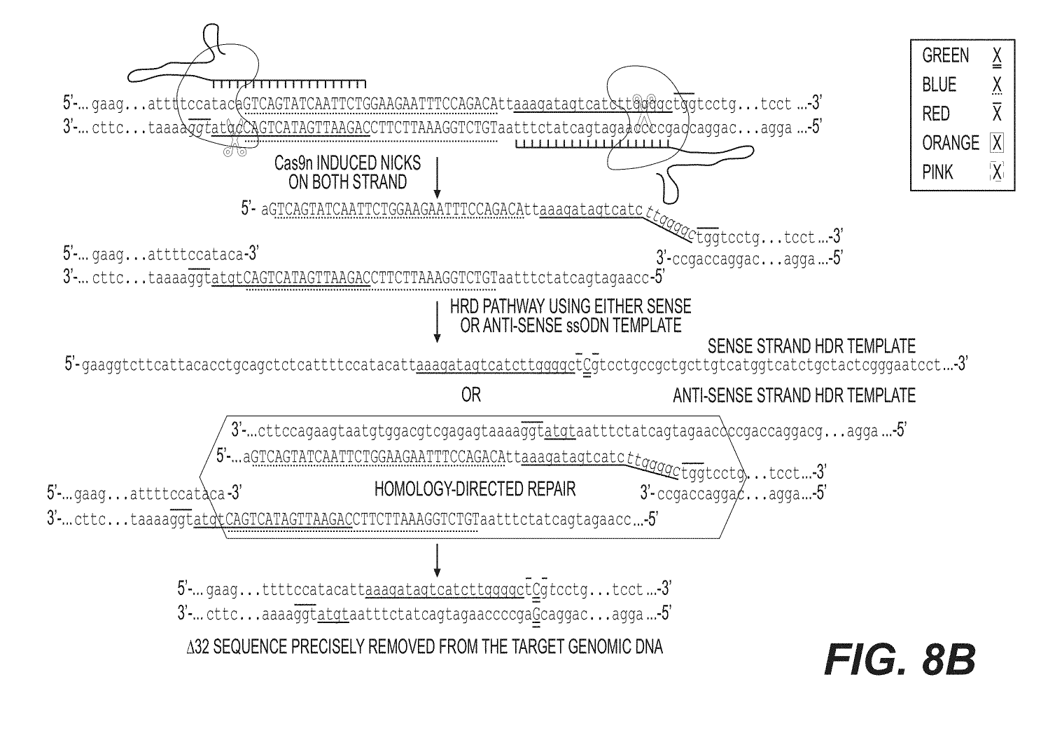

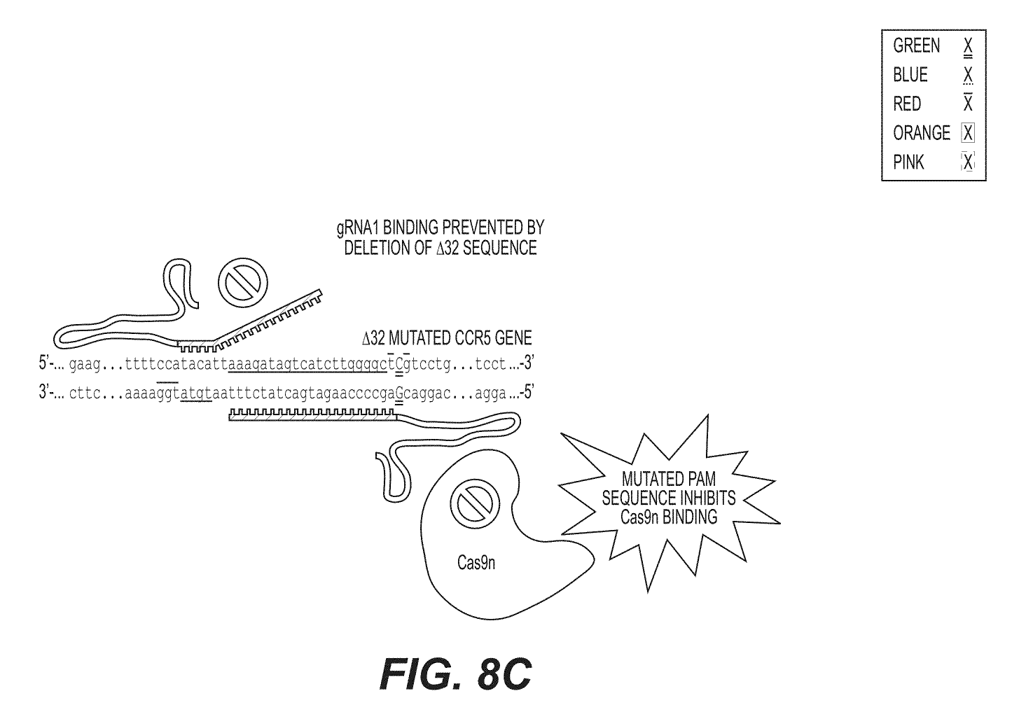

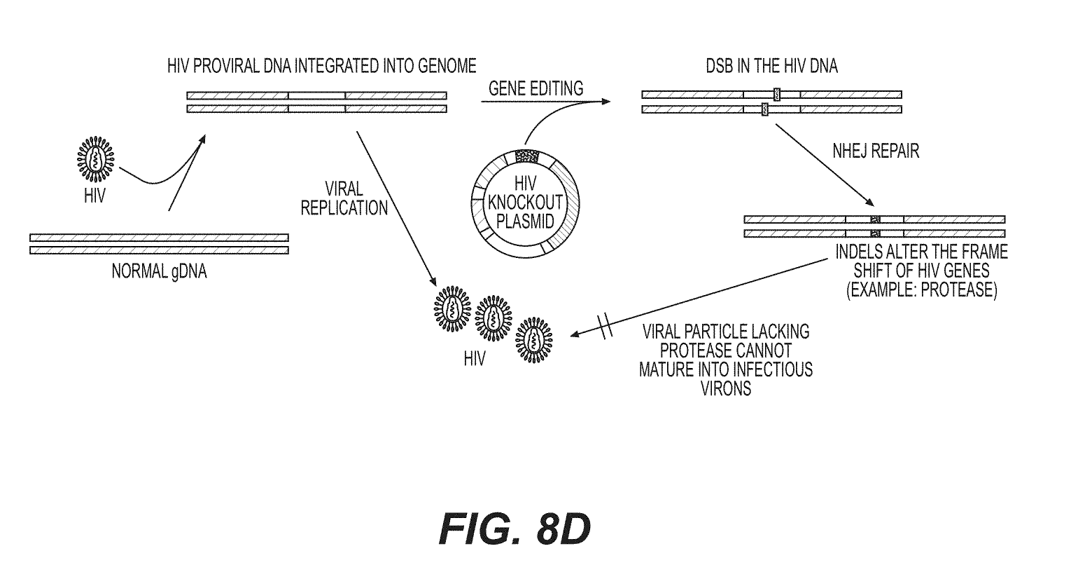

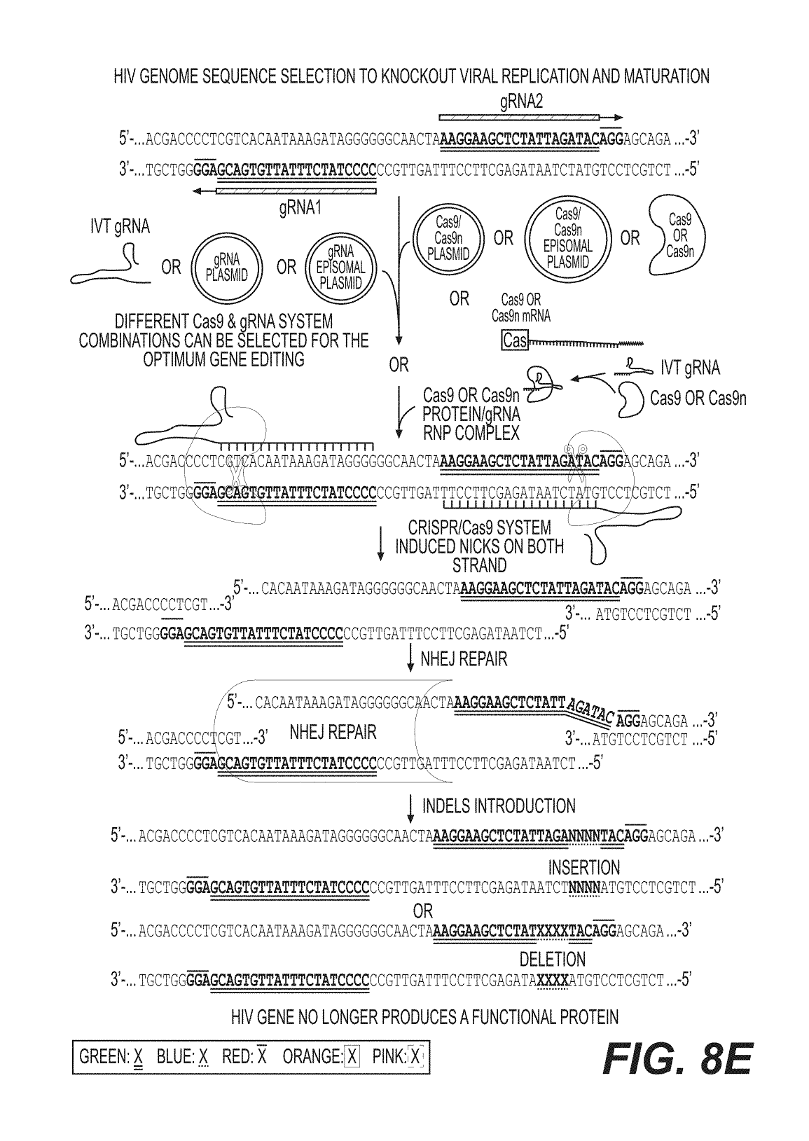

We use CCR5 as an example to illustrate the separation of CRISPR/Cas9 edited CCR5 receptor knockout cells by negative selection. CCR5 belongs to a family of G-protein-coupled receptors and spans the plasma membrane seven times in a serpentine manner. CCR5 serves as a receptor for several chemokines including MIP-1α, MIP-1β, and MCP-2. It also functions as the primary co-receptor for macrophage-tropic HIV-1, which binds to CCR5 through gp120. Extracellular domains of CCR5 are important for HIV entry into target cells. FIG. 8 illustrates a knockout strategy for CCR5 receptor to develop HIV-resistant cells. The CRISPR/Cas9 gene editing strategy shown induces a CCR5Δ32 mutation to pre-terminate CCR5 translation in order to knockout the native CCR5 receptor in the outer membrane of the cells. Edited cells will be purified by tagging the cells of interest with biotinylated primary CCR5 antibody (#ABIN741377, Antibodies Online) followed by the addition of magnetically labeled Anti-Biotin ultrapure microbeads (#130-105-637, Miltenyi Biotec). Then, the cell suspension is loaded onto a MACS Column (#130-042-301, Miltenyi Biotec), and placed in the magnetic field of a MACS Separator (#130-042-102, Miltenyi Biotec). The magnetically labeled material will be retained within the column. Anti-Biotin ultraPure microBeads have the advantage of not binding to free biotin, which will be present in the culture media. The cells edited by the CRISPR/Cas9 system targeting the CCR5 gene will lack the CCR5 receptor on the cell surface and will thus fail to attach to the magnetic beads. The unlabeled CCR5 negative cells thus run through the column and are eluted as the negatively-selected cell fraction. After removing the column from the magnetic field, the magnetically retained material (i.e., the CCR5 positive cell fraction) can be discarded. To increase the purity of the negatively-selected cell fraction, the flow-through cells are passed through a fresh column again as per the procedure mentioned above. The same or similar principles/procedures can be used for the purification of any CRISPR edited cells.

### Example 4. Demonstration of Genetic Manipulation Strategy

Manipulating a target genome sequence using CRISPR/Cas systems provided herein can have a wide range of therapeutic applications, including without limitation: correction of mutated sequences or base pairs in the genome; deletion or insertion of sequences/bps; and induction of mutations, e.g., for transcriptional activation or repression of a gene of interest. Here we use ALS as a genetic disease model to demonstrate gene-editing strategies for correcting genetic mutations associated with ALS.

ALS is the third most common neuromuscular disease worldwide that attacks nerve cells responsible for controlling voluntary muscles. There are currently no definitive diagnoses or effective therapies for ALS. About ˜90% of ALS occurs sporadically without clear associated risk factors and only ˜10% of ALS cases have been found to be familial, being caused by mutations in more than a dozen genes. The familial form of ALS usually results from a pattern of inheritance that requires only one parent to carry the gene responsible for the disease. About 50% of familial ALS cases result from a defect in genes encoding chromosome 9 open reading frame 72 (C9ORF72), which has unknown gene function, and/or superoxide dismutase 1 (SOD1). In the general population, the C9ORF72 gene typically contains from about 3 to 30 GGGGCC hexanucleotide repeats. This number of repeats is considered to be healthy. ALS is associated with heterozygous GGGGCC hexanucleotide expansion of from about 200 to 4500 repeats in a non-coding region of the C9ORF72 gene. These hexanucleotide repeats are believed to be responsible for the disease.

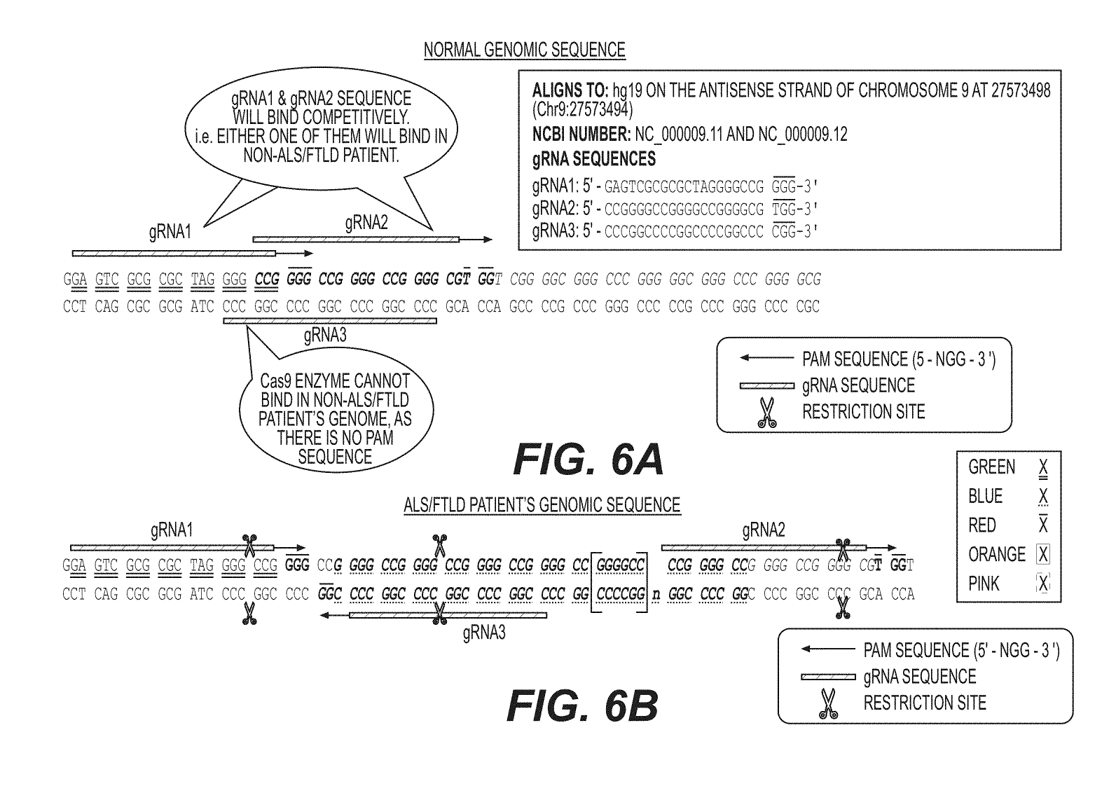

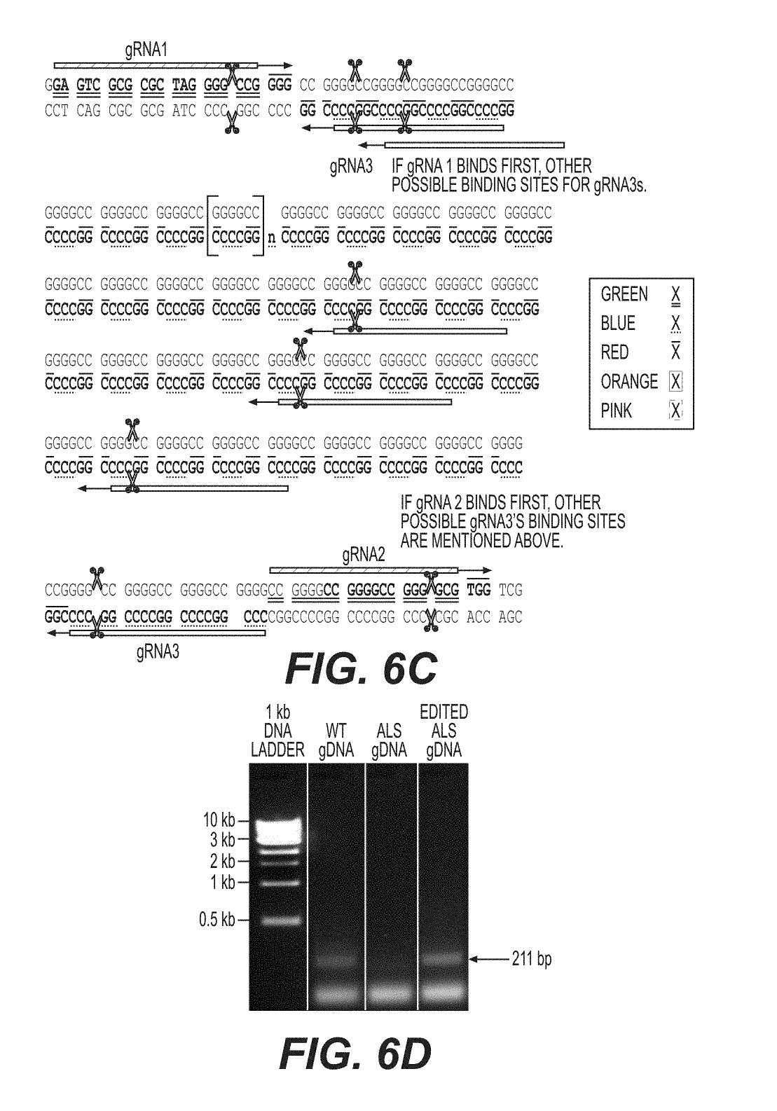

We generate and use a CRISPR/Cas9-based gene editing system using the cellular NHEJ pathway to excise the extra GGGGCC hexanucleotide repeats from the genome of an ALS patient. Through competitive binding of gRNAs and the Cas9n nuclease, the genome of an ALS patient having more than 5 hexanucleotide repeats is selectively modified, whereas the CRISPR gene editing system does not act on a healthy genome having 3 or fewer hexanucleotide repeats (FIG. 6). FIG. 6 shows a schematic illustration for selective DSB in an ALS genome that contains more than five GGGGCC hexanucleotide repeats, using triple gRNAs to guide the Cas9n nuclease and using a NHEJ cellular repair mechanism (FIG. 6B). In contrast, triple gRNA-guided Cas9n nuclease cannot bind to a normal genomic DNA having three GGGGCC hexanucleotide repeat sequences (FIG. 6A), suggesting that our CRISPR system designed with triple gRNA and Cas9n nuclease can potentially remove the heterozygous hexanucleotide expansion without disturbing another normal locus.

Triple gRNA guided excision of extra GGGGCC hexanucleotide repeats in C9ORF72 is validated by a Loop-mediated isothermal amplification (LAMP) technique and diagnostic ALS PCR. LAMP is an auto-cycling strand displacement DNA synthesis procedure, carried out by Bst DNA polymerase with high strand displacement activity and a set of four specially designed primers that recognize a total of six distinct sequences on the target DNA (Notomi, T. et al., Nucleic Acids Res. 28, e63, 2000). LAMP is therefore expected to amplify the target sequence with high selectivity (Notomi, T. et al., Nucleic Acids Res. 28, e63, 2000; Nagamine, K. et al., Mol. Cell. Probes 16:223-229, 2002). We design a set of four primers that specifically bind to the C9ORF72 gene that has extra GGGGCC hexanucleotide repeats; these primers fail to recognize and amplify normal genomic DNA or the ALS-C9ORF72 gene in which the extra GGGGCC hexanucleotides have been excised. The final LAMP-amplified GGGGCC hexanucleotide-containing C9ORF72 gene products are a mixture of stem-loop DNA of various lengths. LAMP amplified products can be analyzed by real-time PCR (with real-time probes), turbidity, fluorescent assay and agarose gel electrophoresis. When LAMP products are visualized by agarose gel electrophoresis, many bands of different sizes up to the loading well are seen.

PCR amplification of the targeted gDNA region and the endonuclease assay are the conventional methods that have been developed to detect the efficiency of indel mutations induced by CRISPR/Cas9 activity. The extra GGGGCC hexanucleotide repeat-containing C9ORF72 gene amplification is technically challenging due to the presence of a high percentage of GC base pairs. A qualitative PCR method to validate the presence of extra GGGGCC hexanucleotide repeats in the C9ORF72 gene was therefore developed. Briefly, the PCR amplification was carried out using Phusion™ High-Fidelity PCR Master Mix (ThermoFisher, MA, USA). The forward primer NWL-MBPr-664 (5′-GGGTCTAGCAAGAGCAGGTGTGGGTTTAGGAGGTGTGTG-3′) and reverse primer NWL-MBPr-674 (5′-GCCCCGACCACGCCCCGGCCCCGGCCCCGGCCCCTAGCG-3′) were used to amplify a 211-bp PCR product from both the wild type (WT) and extra GGGGCC hexanucleotide repeat-containing C9ORF72 gene. The PCR cycling parameters used with the C1000 Thermal Cycler (Bio-Rad, CA, USA) were as follows: initial denaturation at 98° C. for 3 min, 32 cycles of 98° C. for 30 seconds, 71° C. for 30 seconds, 72° C. for 60 seconds, and final extension at 72° C. for 5 min. After amplification, the PCR products were resolved by 1.5% agarose gel electrophoresis. Visualizing the PCR products by agarose gel electrophoresis showed the following: WT resulted in a high-intensity band, while any extra GGGGCC hexanucleotide repeat-containing C9ORF72 sequence resulted in a low-intensity band. We utilized the band intensity difference in diagnostic ALS PCR to screen the extra GGGGCC hexanucleotide repeat excisions.

Human Neural Stem Like-Cells (NSLC) from an ALS patient were cultured in neural proliferation media (NeuroCult™ proliferation medium, STEMCELL Technologies, Vancouver, BC, Canada) supplemented with EGF (20 ng/ml, Peprotech, QC, Canada) and FGF (20 ng/ml, Peprotech) at 37° C., 5% CO₂, 5% O₂ until 80% confluency. After reaching the desired confluency, the cells were harvested with TrypLE™ (Life Technologies, CA, USA) by incubating the cells in TrypLE for 3 to 5 minutes at 37° C. The cells were pelleted by centrifugation at 1500 rpm for 5 minutes and the cell pellet was used for transfection experiments. About 1×10⁶ cells were gently re-suspended in 100 μl of P3 solution (Lonza) and combined with 2 μg of pD-Epi723gRNA1 plasmid and 1.5 μg of Cas9n mRNA. This mixture was then transferred to a nucleofection cuvette and the cells were transfected using the program “DS150” in a 4D-Amaxa Nucleofector™ Device (Lonza, Walkersville, Md., USA). The transfected cells were transferred to a laminin-coated 6-well culture plate seeded at a cell density of ˜2×10⁵/well and incubated overnight in the neural proliferation media supplemented with EGF (20 ng/ml) and FGF2 (20 ng/ml) at 37° C., 5% CO₂, 5% O₂. The un-transfected cells were also plated at the same cell density as negative control for this experiment. After the overnight incubation, the media was replaced with fresh neural proliferation media supplemented with EGF (20 ng/ml) and FGF2 (20 ng/ml). The transfected cells were subsequently re-transfected two more times (3 days apart) with 1.5 μg of Cas9n mRNA using the Lipofectamine™ MessengerMAX (Invitrogen) as per the manufacturer's protocol. The triple transfected cells were further cultured in the neural proliferation media supplemented with EGF (20 ng/ml) and FGF2 (20 ng/ml) for another 48 hours and then collected for diagnostic ALS PCR analysis. Diagnostic ALS PCR was performed to examine whether the pD-Epi723gRNA1 plasmid/Cas9n mRNA removed the extra GGGGCC hexanucleotide repeats in the ALS-C9ORF72 gene. As shown in FIG. 6D, a triple gRNA based CRISPR/Cas9-based gene editing system was able to efficiently excise the extra GGGGCC hexanucleotide repeats from the genome of an ALS patient with the C9ORF72 mutation.

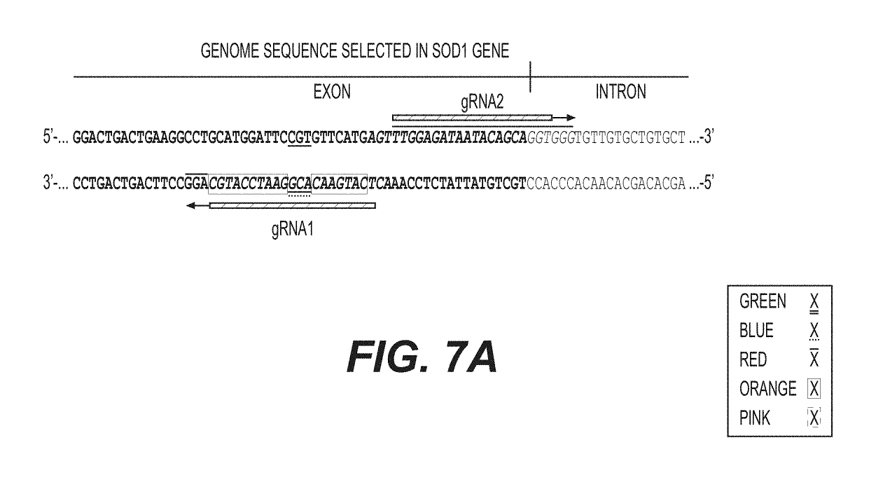

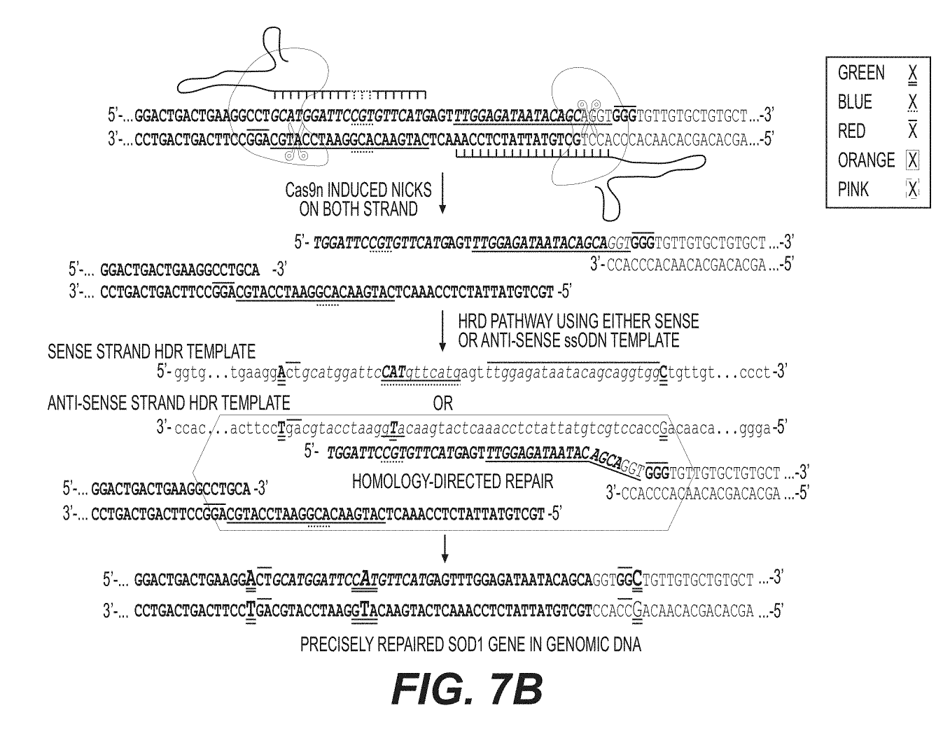

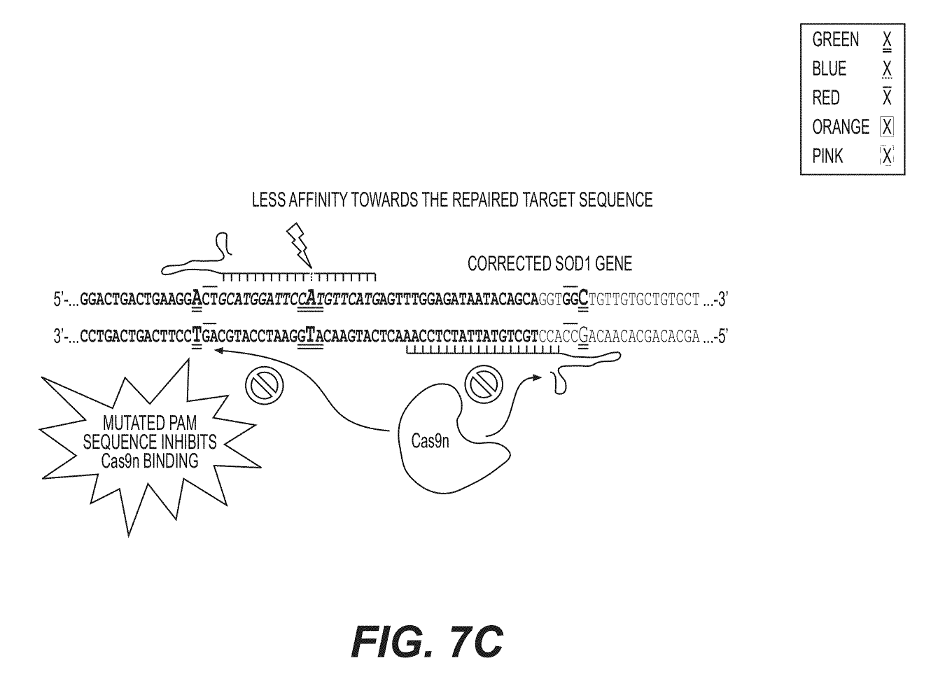

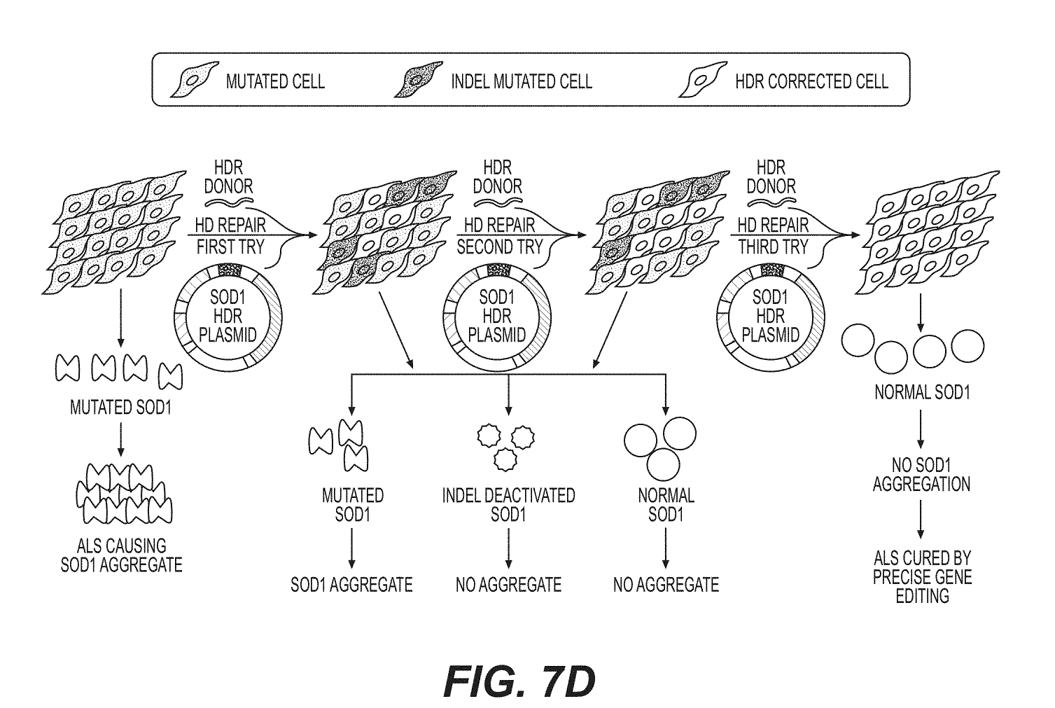

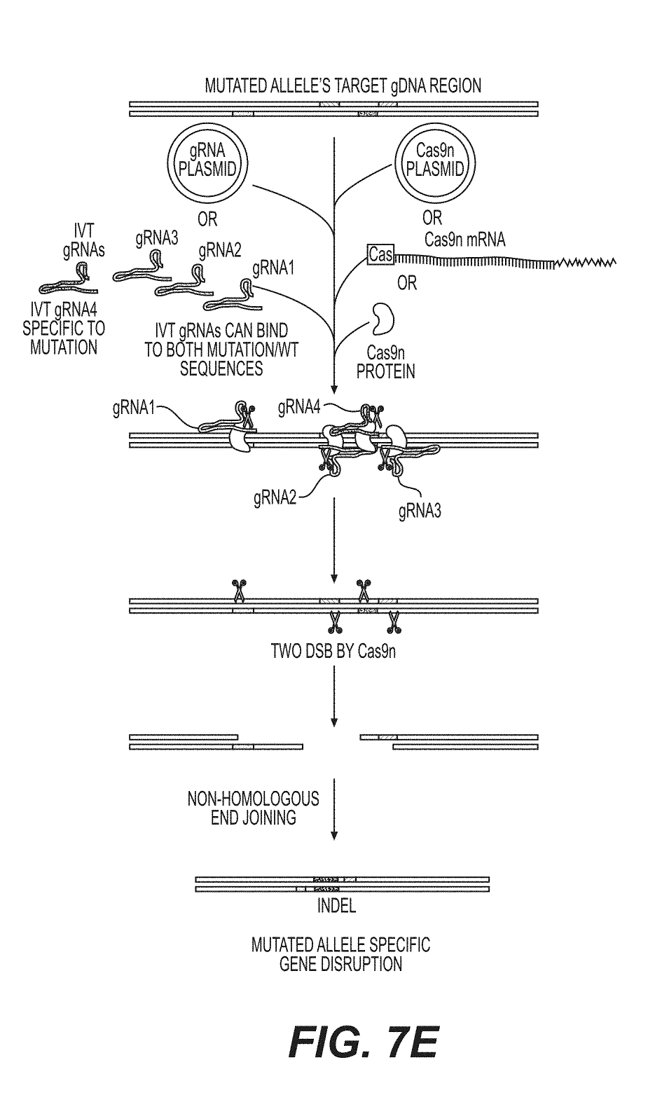

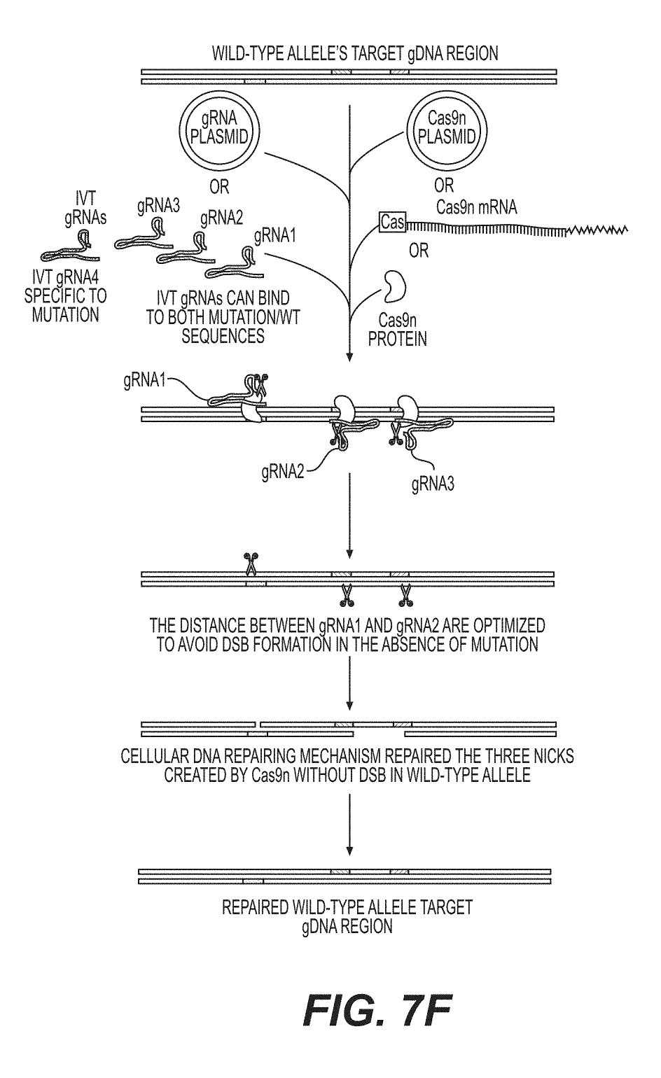

At present more than 150 different mutations in the SOD1 gene have been identified in ALS patients. H46R, a mutation in the 46^{th} codon for histidine changed to arginine, is the most common ALS-causing mutation. H46R causes a profound loss of copper binding to the SOD1 active site, which renders SOD1 enzymatically inactive. We generate a CRISPR/Cas9-based gene editing system using a ssODN-directed HDR pathway to site specifically correct the H46R mutated codon. FIG. 7 illustrates the design scheme for gRNA and ssODN to correct the arginine at the 46^{th} position of SOD1 to histidine.

We also design a correcting ssODN template with a histidine codon at the 46^{th} position for HDR targeting of the non-coding strand of the gene. Since the non-coding strand has been shown to be highly specific and does not interfere with transcription and gene expression, the specificity of the ssODN is confirmed to avoid the ssODN HDR template targeting other sequences in the genome. To improve stability of the ssODN HDR template, we incorporate a tag at the 3′ end (such as, but not limited to, a CGCG repeat of phosphorothioate) to increase intracellular stability towards endonuclease and exonuclease. To improve efficiency of the ssODN HRD template, a peptide nucleic acid at the end of the ssODN consisting of nucleic acid bases attached to an archiral peptide backbone made up of N-2-aminoethyl glycine units is incorporated. Furthermore we can incorporate a tracking fluorophore (such as, without limitation, a Cyanine dye or a quantum dot) at the 5′ end of the ssODN to allow monitoring of its cellular uptake and distribution. The PAM sequences in the ssODN HDR donor are also masked silently (without affecting the amino acid sequence) to avoid degradation by Cas9n nuclease and to avoid repeated genetic modification after the desired modification has taken place. Both the above-mentioned gene editing methodologies efficiently correct the genetic disorders and retain target gene function.

We also use similar methods to introduce a CCR5 Δ32 mutation into a cell. CCR5 is a co-receptor for CD4, which plays a critical role in the entry of HIV into host CD4⁺ cells). Cells with a CCR5 Δ32 mutation are known to be resistant to HIV entry (see FIG. 8).

### Example 5. Therapeutic Applications of HDR-Mediated Genome Editing for Mitrochondrial Disease

Mitochondria are double membrane sub-cellular organelles present in all mammalian nucleated cells. Their main role is to produce cellular ATP through oxidative phosphorylation. Mitochondria have their own DNA, which is distinct from the chromosomal DNA present in the nucleus. Human mitochondrial DNA is a small circular double stranded DNA of about 16.6 kb in size, encoding 13 essential polypeptides, which are critical for oxidative phosphorylation. Mitochondria replicate their DNA by themselves. Harmful mutations in mitochondrial DNA can cause a number of serious diseases. The rate of mitochondrial DNA mutation is 10 to 17-fold higher than nuclear DNA mutation. Unlike mutations in nuclear DNA, which are inherited from both parents, mitochondrial mutations are inherited only from the mother.

Two types of mutations, namely point mutations and rearrangements, occur in mitochondrial DNA. Over 250 harmful mitochondrial mutations have been characterized (MITOMAP: A Human Mitochondrial Genome Database, http://www.mitomap.org, 2009). These mutations disrupt the mitochondria's ability to generate energy efficiently. Mitochondrial dysfunction may lead to several diseases in many organs such as progressive myopathy, cardiomyopathy, retinitis pigmentosa, Leber hereditary optic neuropathy (LHON), progressive brain-stem disorder, diabetes, MELAS (mitochondrial encephalomyopathy, lactic acidosis, and stroke-like episodes), and so on. Currently there are no effective treatments for the majority of mitochondrial diseases.

Due to the complexity of the mitochondrial DNA location, certain modifications are required in nuclear CRISPR/Cas9-mediated gene editing methodology to precisely edit the mutated mitochondrial DNA, including for efficiently targeting the Cas9 enzyme and HDR donor ssODN towards mitochondrial DNA. These modifications include adding a mitochondrial targeting sequence (MTS), such as the MTS from Ornithine transcarbamylase or cytC, at the N-terminal end to direct the Cas9 enzyme towards the mitochondrial matrix, and modifying the donor ssODN at its 3′ end by adding peptide nucleic acids (PNA) attached with either a mitochondrial signal peptide (MSP) or triphenylphosphonium (TPP). The mechanisms of MSP and TPP targeting of the ssODN to the mitochondrial matrix are unique, as MTS is recognized by mitochondrial surface receptors and TPP can easily pass through the phospholipid bilayer towards the negatively charged mitochondrial matrix (Yoon, Y. G. et al., Anat. Cell Biol. 43 (2): 97-109, 2010). These unique properties of the conjugates promote efficient delivery of the ssODN donor to precisely edit the mitochondrial DNA mutations.

Selective repair of mutant mitochondrial DNA without affecting normal or wild type mitochondrial DNA is of great importance. The localized oxidative environment and increased replication in the mitochondria can sometimes make mitrochondrial DNA mutation more frequent. In such conditions mutant mitochondrial DNA co-exist with normal or wild type DNA in various proportions, referred to as heteroplasmy. Thus an increase in the proportion of mutant mitochondrial DNA is required for a disease to be expressed or to increase disease severity. To selectively repair the mutant DNA and increase the proportion of wild type mitochondrial DNA for complete recovery from the disease phenotype, an HDR donor ssODN that is linked with either PNA-MSP or PNA-TPP along with Cas9 enzyme and gRNA (Cas9+gRNA)-expressing plasmid can be used. It should be understood that this configuration is used as an example only; many other configurations are also possible, as described herein, and are intended to be encompassed. For example, episomal expression of the gRNA in the cells may be introduced first, followed by the Cas9 protein and the modified ssODN. The gRNA is designed so that it exclusively binds to mutated mitochondrial DNA, but not to wild type DNA. For efficient mitochondrial DNA editing, either co-transfection of donor ssODN and Cas9+gRNA expressing plasmid, or transfection of donor ssODN 24 hours after transfection of Cas9+gRNA-expressing plasmid, can be used.

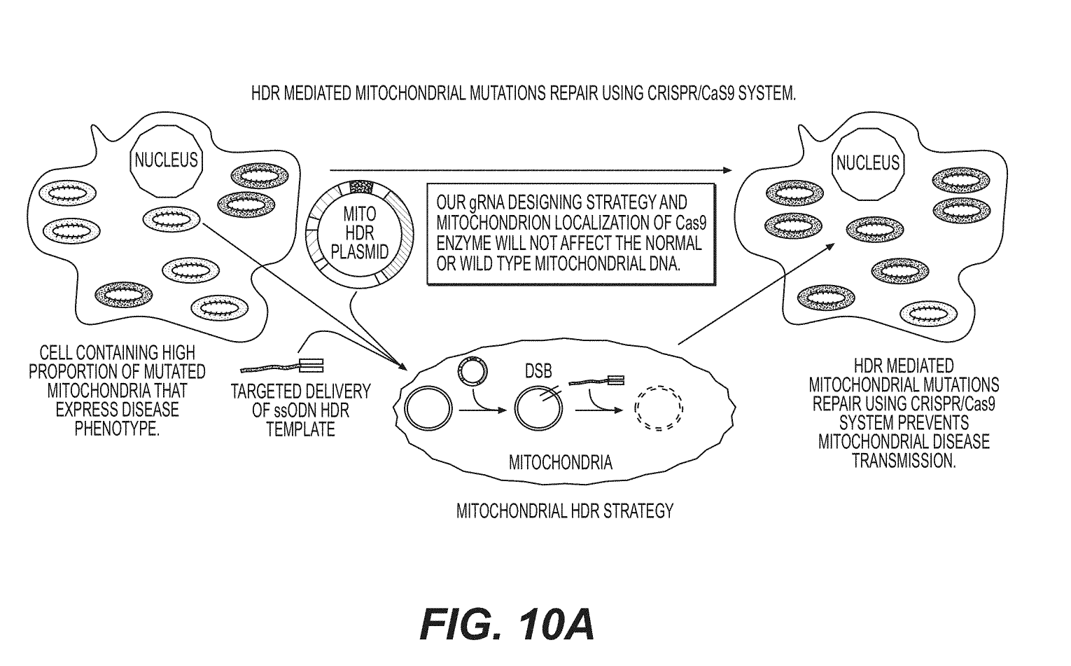

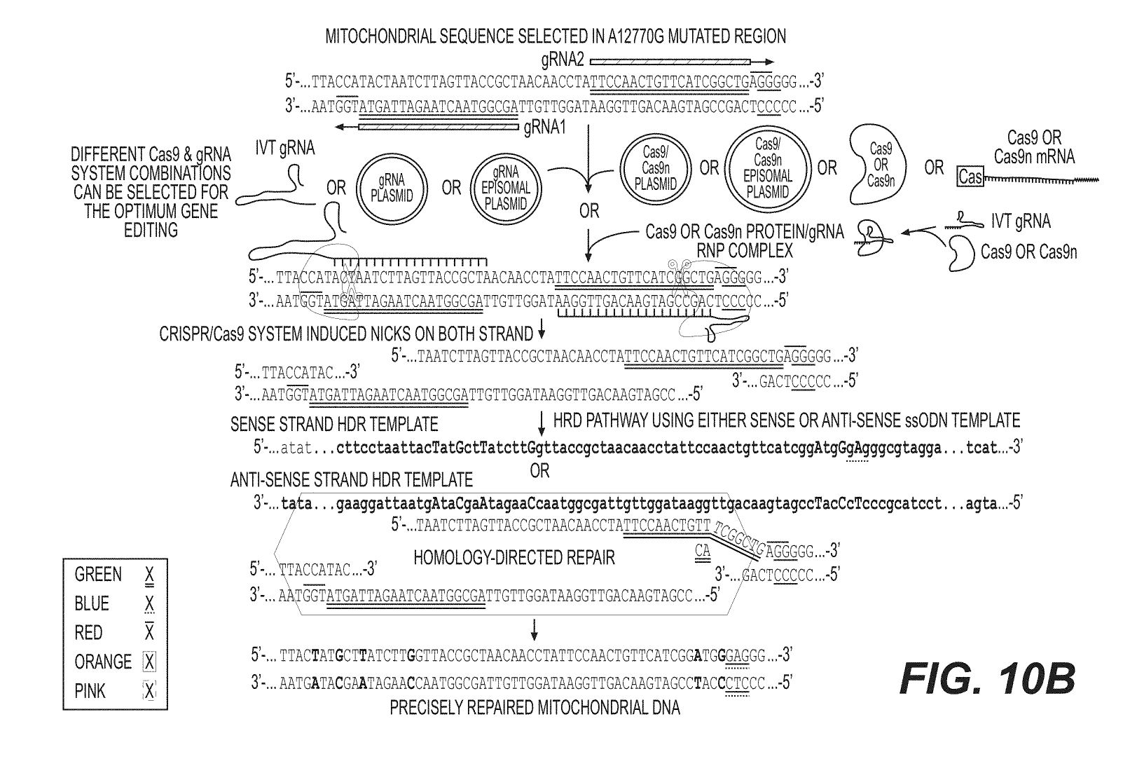

A schematic diagram illustrating such a system for use in correcting mutated mitochondrial DNA targets is shown in FIG. 10, which shows an exemplary system for correcting MELAS. MELAS is one of several diseases associated with mitochondrial cytopathies. MELAS is often progressive and fatal and is caused by defects in the mitochondrial genome, which is maternally inherited. There is no known treatment for MELAS. Management of this disease often depends on what areas of the body are affected at a particular time. FIG. 10 details the design of two gRNAs and ssODN conjugated with either MSP or TPP to correct the nt.A12770G mitochondrial mutation. Similar systems can be used to provide permanent recovery from any mitochondrial disease caused by mutations in the mitochondrial DNA.

### Example 6. Functional Deletion of Pathogenic Mutant Gene Expression by Indel Mediated Gene Editing

Correcting mutations via high fidelity HDR pathways with a donor template is generally less efficient than non-homologus end joining (NHEJ), which can repair a DSB by directly rejoining the two ends in a process that does not require any homology repair template. During the repair process, NHEJ introduces indels that can cause frame shift mutations in the target gene and lead to mRNA degradation by nonsense-mediated decay or result in the production of truncated non-functional proteins. Mutations in several proteins cause pathogenic abnormal functionality that could be more virulent than complete knockout of such genes. In such cases, complete deletion of a pathogenic mutated gene may cause less harm than the expression of the pathogenic form of that specific protein.

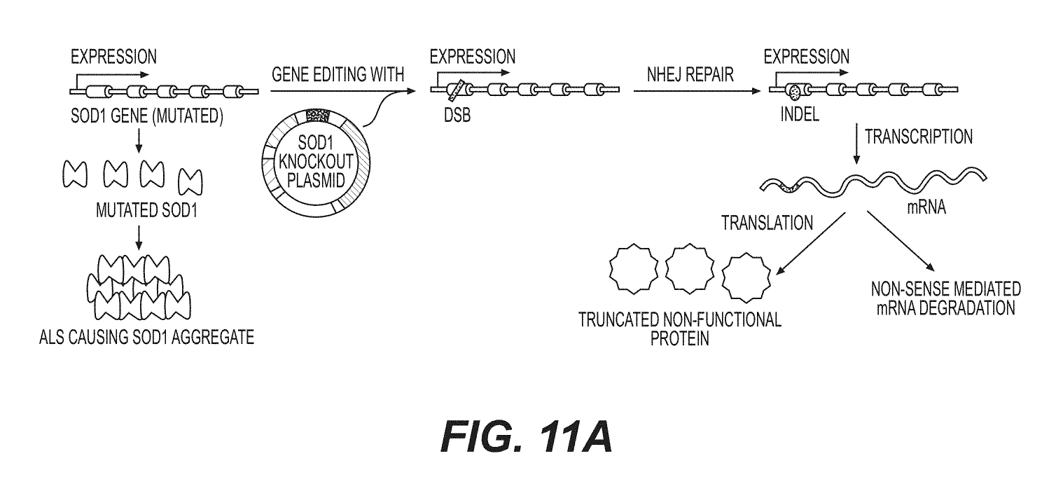

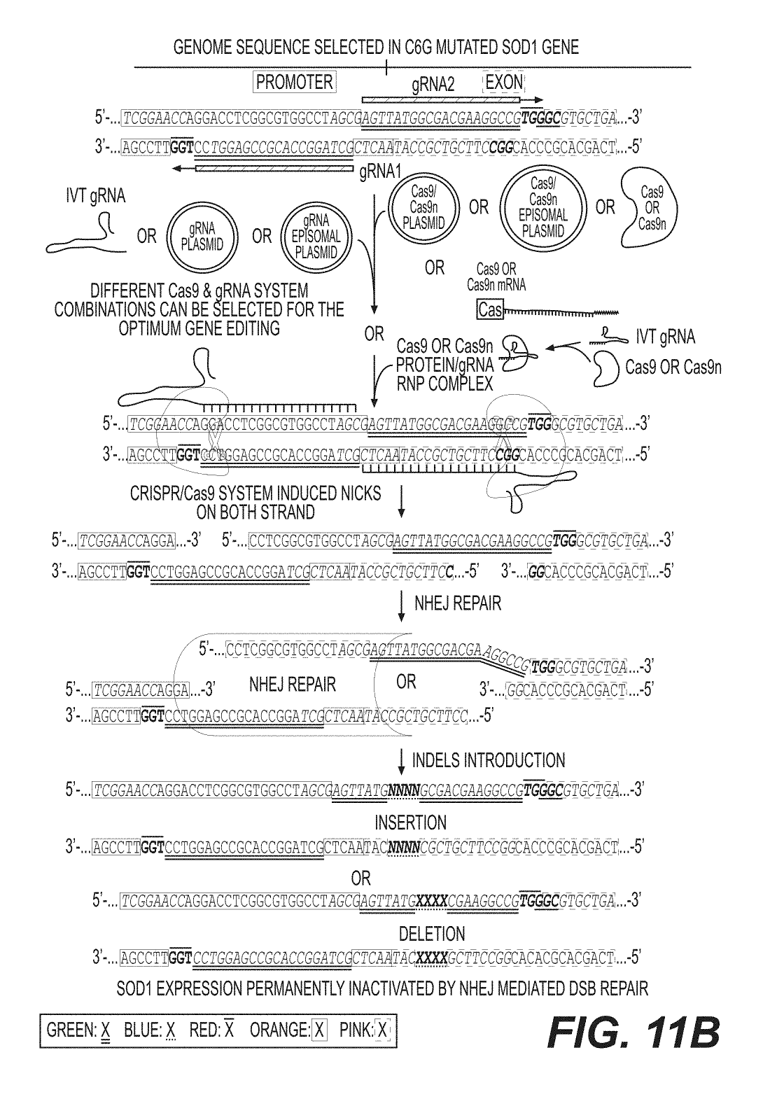

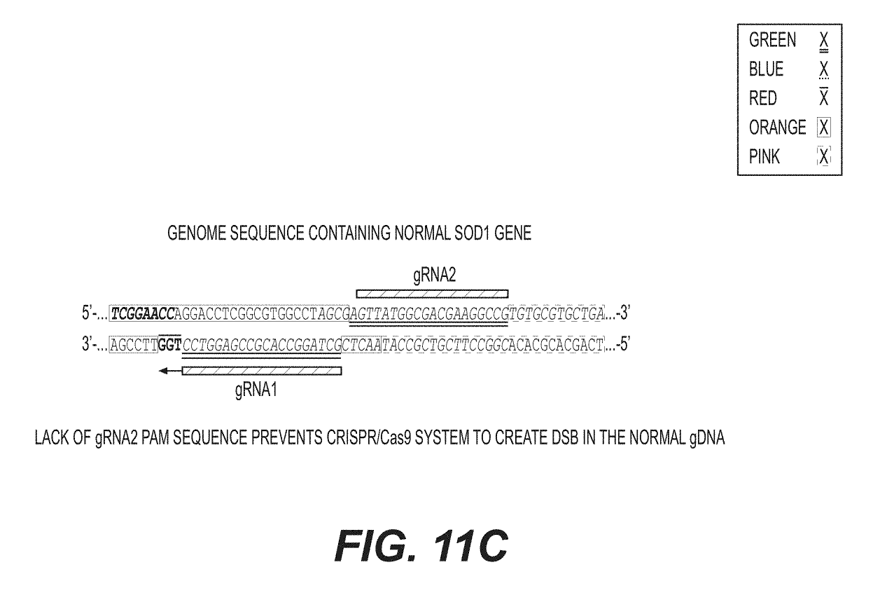

For illustrative purposes, SOD1 is used here as an example. Mutations in the SOD1 gene have been found in about 12-13% of familial cases of amyotrophic lateral sclerosis (ALS). Currently, about 150 different mutations have been reported in the SOD1 gene that causes ALS. ALS is a protein misfolding disease. Mutations in the SOD1 gene cause an increased propensity to form aggregates that may confer toxicity, especially in motor neurons. Minute amounts of mutated SOD1 aggregates are sufficient to act as prions in transmitting a templated, spreading aggregation of SOD1, that leads to development of fatal ALS (Bidhendi, E. E. et al., J. Clin. Invest. 2016 (in press), doi: 10.1172/JCI84360). It has been confirmed that motor neuron disease caused by the SOD1 protein is due predominantly to the gain of such toxic properties and not through loss of function. Hence, we have developed a gene editing strategy to completely delete the expression of the SOD1 enzyme, instead of merely correcting specific mutations. FIG. 11 shows a schematic diagram illustrating the complete deletion of pathogenic mutant protein expression. The same or similar methods can be applied to treat similar diseases caused by pathogenic mutant protein expression.

### Example 7. Other Diseases Targeted by Similar Gene Editing Strategies

Gene editing systems and methods described herein can be used not only for treating diseases caused by genetic mutations, but also for treating infectious diseases and diseases that having both genetic and environmental components. For example, systems and methods described herein can be used to specifically knockout genes required for the survival of pathogenic human infectious agents, such as viruses, bacteria, parasites, yeast and prion proteins. Further, systems and methods can be used to knockout a previously inserted transgene in the germ line of a genetically modified organism (GMO). For illustration, we also describe herein the design of NHEJ mediated gene editing strategies to knockout (e.g., inactivate) previously inserted transgenes in the germ line of GMOs.

About 4000 human diseases that are caused by gene disorders have been identified (Online Mendelian Inheritance in Man (OMIM): An Online Catalog of Human Genes and Genetic Disorders, http://www.omim.org; The Human Gene Mutation Database (HGMD®): An attempt to collate known (published) gene lesions responsible for human inherited disease, http://www.hgmd.cf.ac.uk/ac/index.php; Piñero, J. et al., Database (Oxford), 2015; 2015:bav028). Among these genetic disorders, current and potential candidates for gene therapy include, without limitation, cancer, cardiovascular diseases, metabolic diseases, AIDS, cystic fibrosis, amyotrophic lateral sclerosis, Parkinson's disease, Alzheimer's disease, and arthritis. The same or similar systems and methods described herein can be used to treat such genetic disorders.

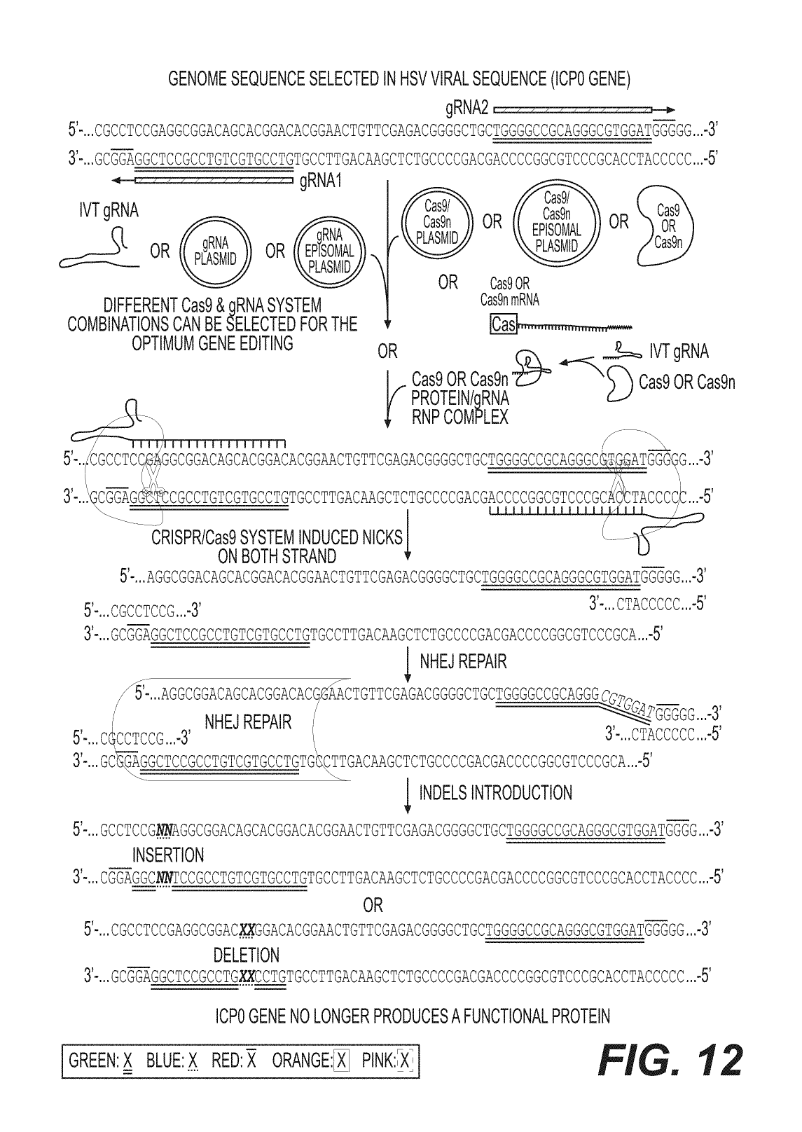

Examples of diseases and their associated infectious agents, along with transgenes, that may be targeted using systems and methods described herein are shown in Table 5, which lists various human disease models and their respective infectious agents. The examples shown in Table 5 are for illustrative purposes only and are not meant to be limiting. Examples are also illustrated in FIGS. 12-19, as indicated.

Additional examples of genetic disorders that may be targeted using systems and methods described herein are shown in Table 6, which also lists exemplary gRNAs. Examples are also illustrated in the Figures as indicated.

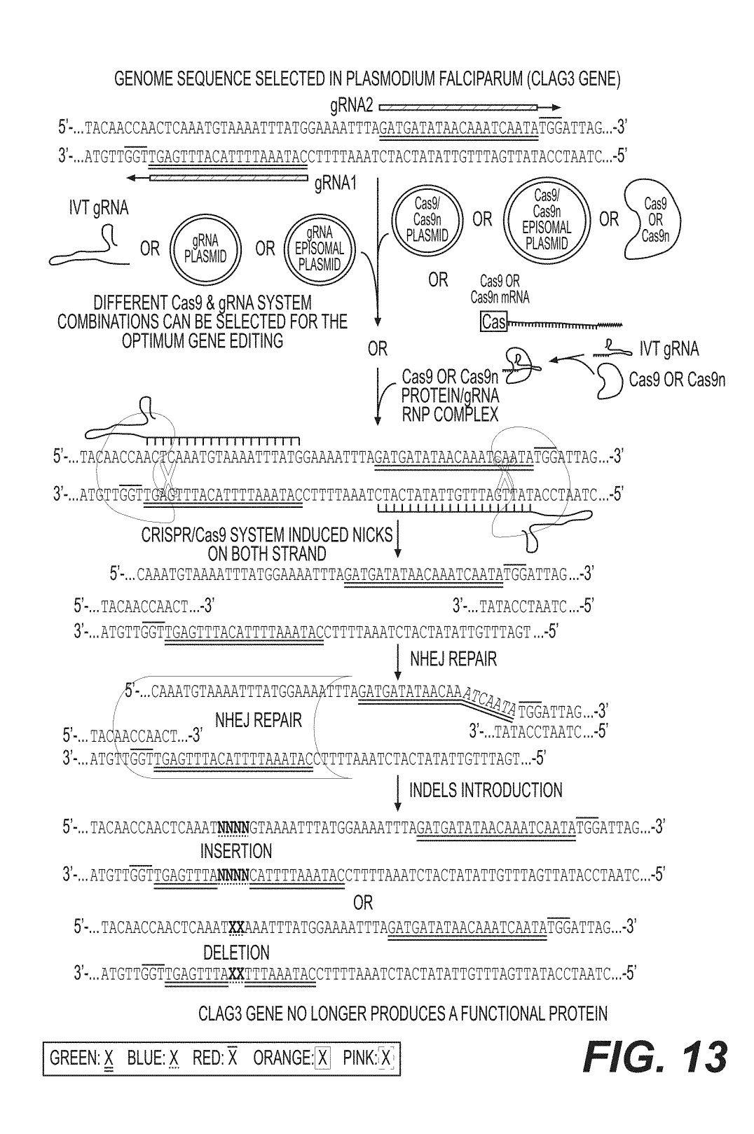

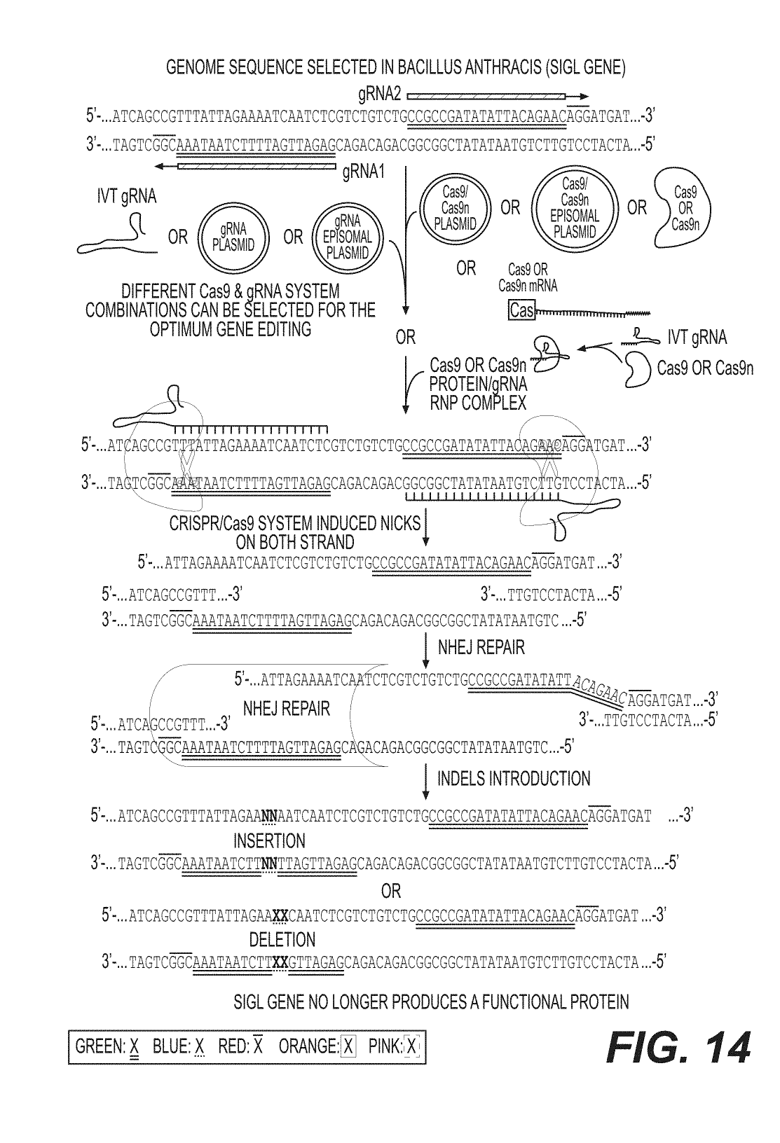

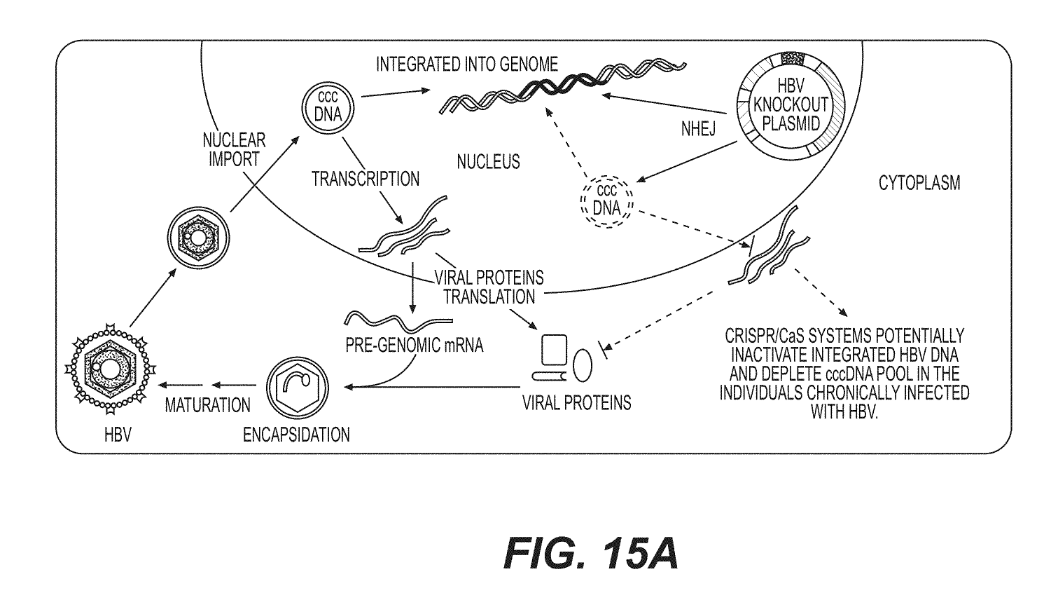

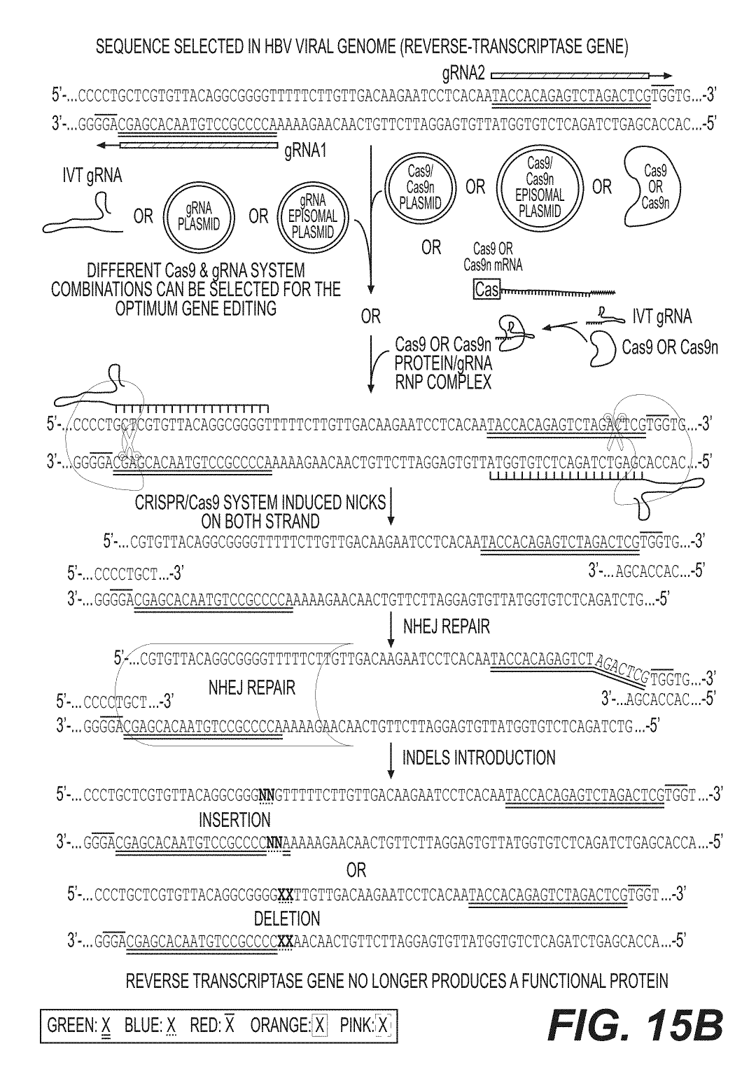

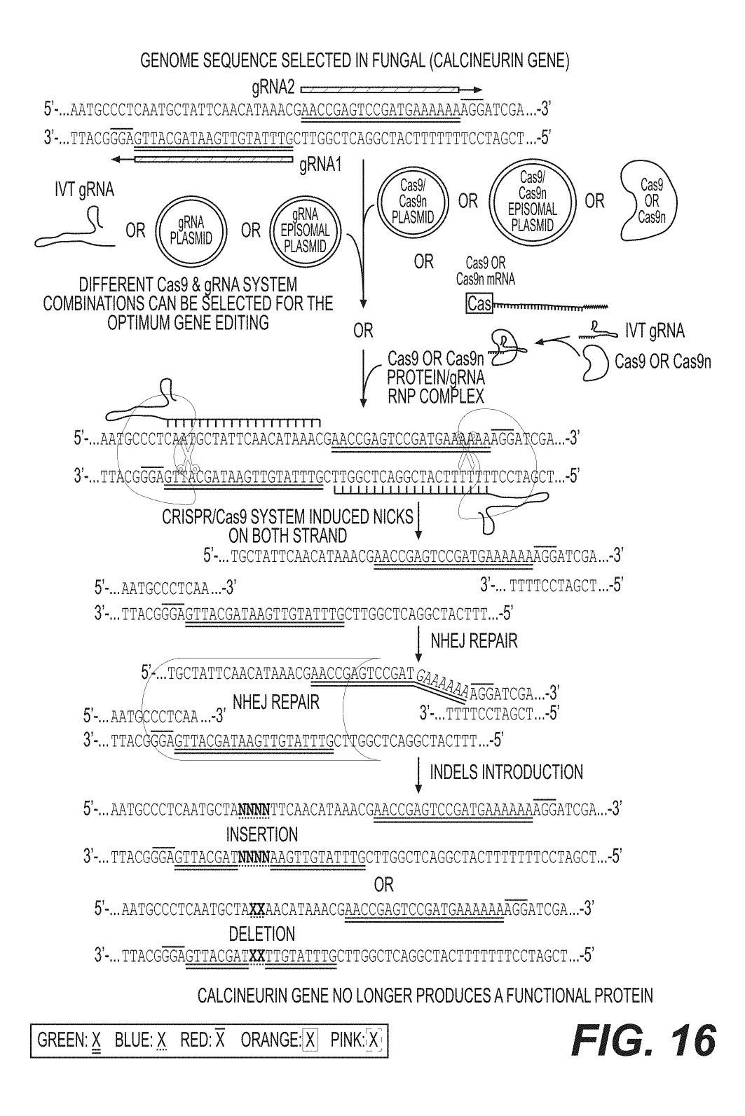

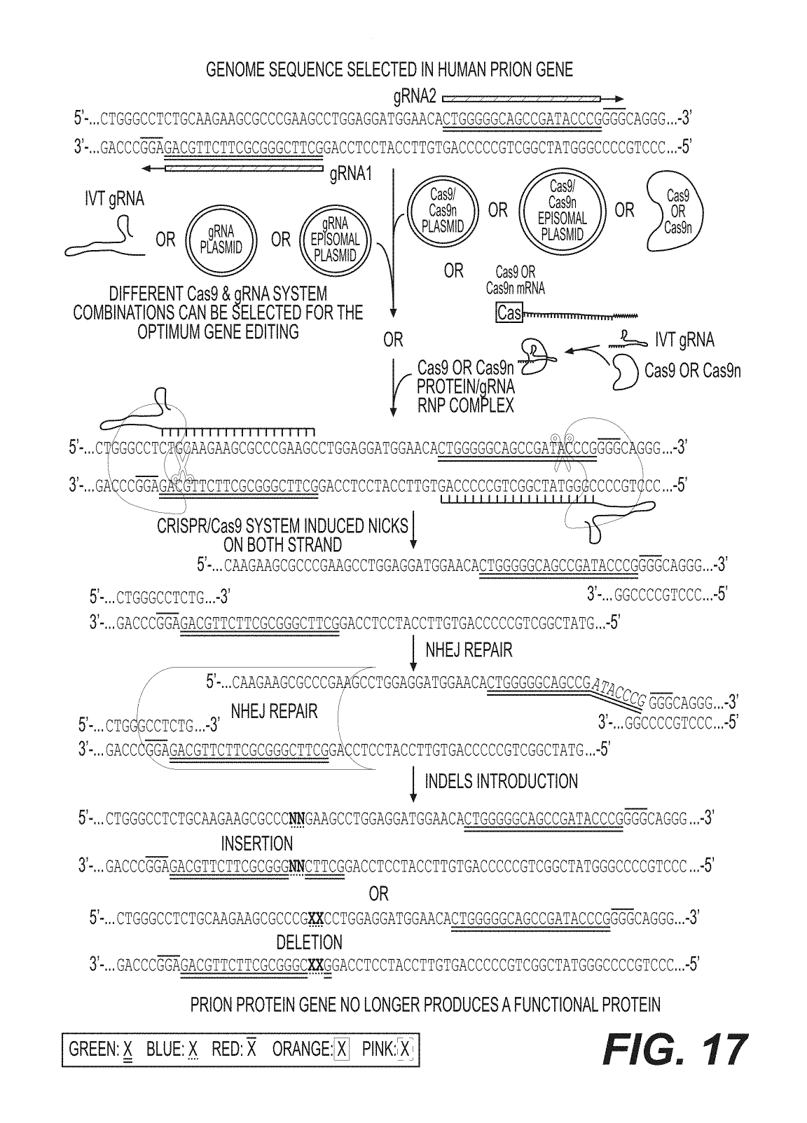

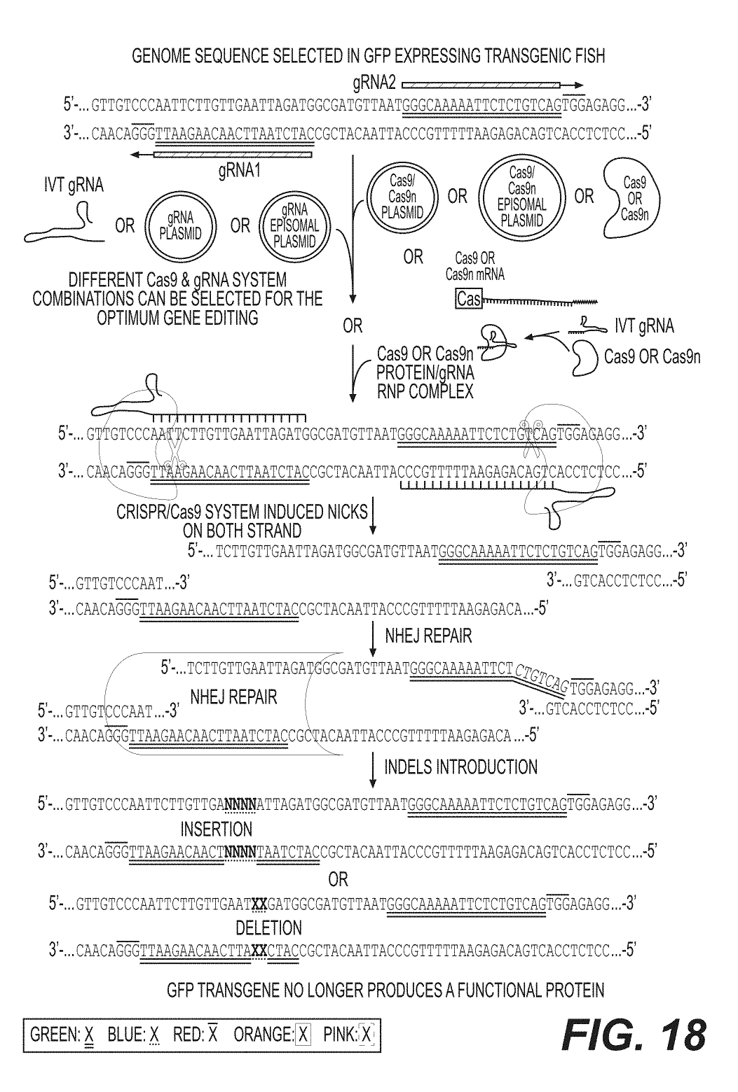

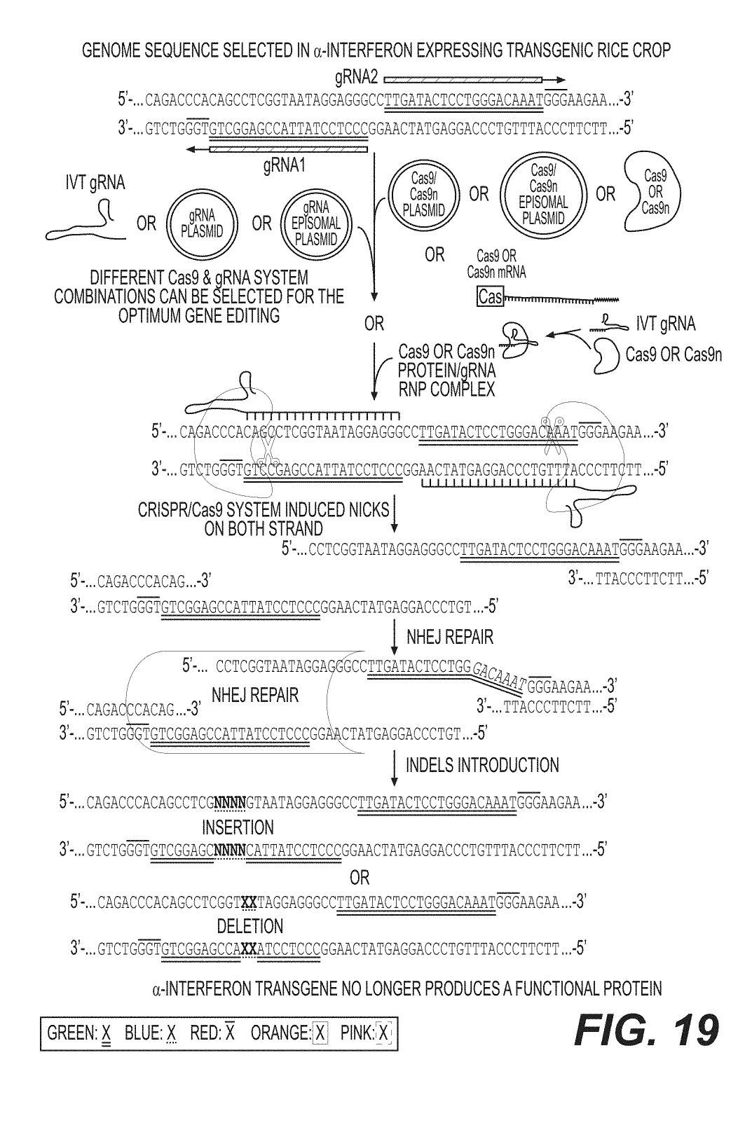

TABLE 5
Exemplary gene targets and their functions for various infectious agents and transgenes.

| | Human | Infectious | | | |
|---|---|---|---|---|---|
| | Disease | Agent/ | Gene | | Reference |
| | Model | Transgene | Targeted | Targeted Gene Function | FIG. |
| 1 | Virus that | Herpes | ICP0 | Stimulates onset of lytic infection and | FIG. 12 |
| | affects | | | productive reactivation of herpes | |
| | neurons | | | genome from latency. | |
| 2 | Parasitic | Plasmodium | Clag3 | Determines channel mediated nutrient | FIG. 13 |
| | disease | falciparum | | uptake by infected RBC for the growth | |
| | | | | and proliferation of plasmodium | |
| | | | | parasites. | |
| 3 | Pathogenic | Bacillus | Sigl | Required for the growth of B. anthracis | FIG. 14 |
| | bacterial | anthracis | | and transcription of toxin genes | |
| | disease | | | expression. | |
| 4 | Pathogenic | Hepatitis B | cccDNA & | Inactivate HBV viral replication in | FIG. 15 |
| | viral disease | virus | integrated | chronically infected patients. | |
| | | | proviral | | |
| | | | genome | | |
| 5 | Pathogenic | Candida | Calcineurin | Permanently halt the growth and | FIG. 16 |
| | yeast | albicans | | virulence of Candida albicans. | |
| | disease | | | | |
| 6 | Prion protein | Prion protein | PRNP | Suppress the conversion of the normal | FIG. 17 |
| | disease | | | prion protein into the pathogenic form | |
| | | | | and slow down or eradicate the | |
| | | | | aggregation of pathogenic prions in the | |
| | | | | brain. | |
| 7 | GMO | GFP | GFP | Inactivates transgene. | FIG. 18 |
| | (animal) | | | | |
| 8 | GMO (plant) | α-interferon | α-interferon | Inactivates transgene. | FIG. 19 |

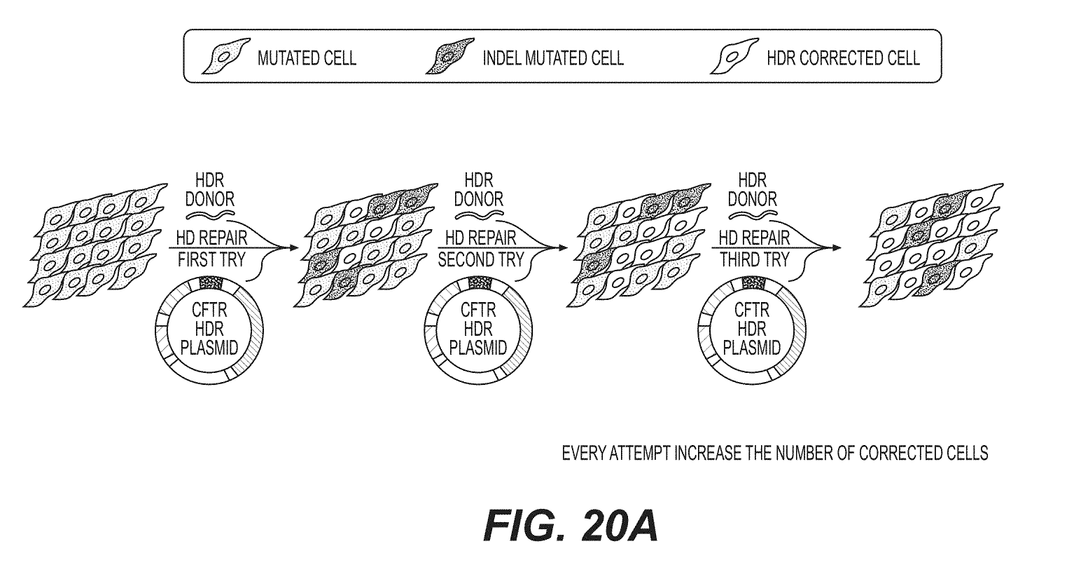

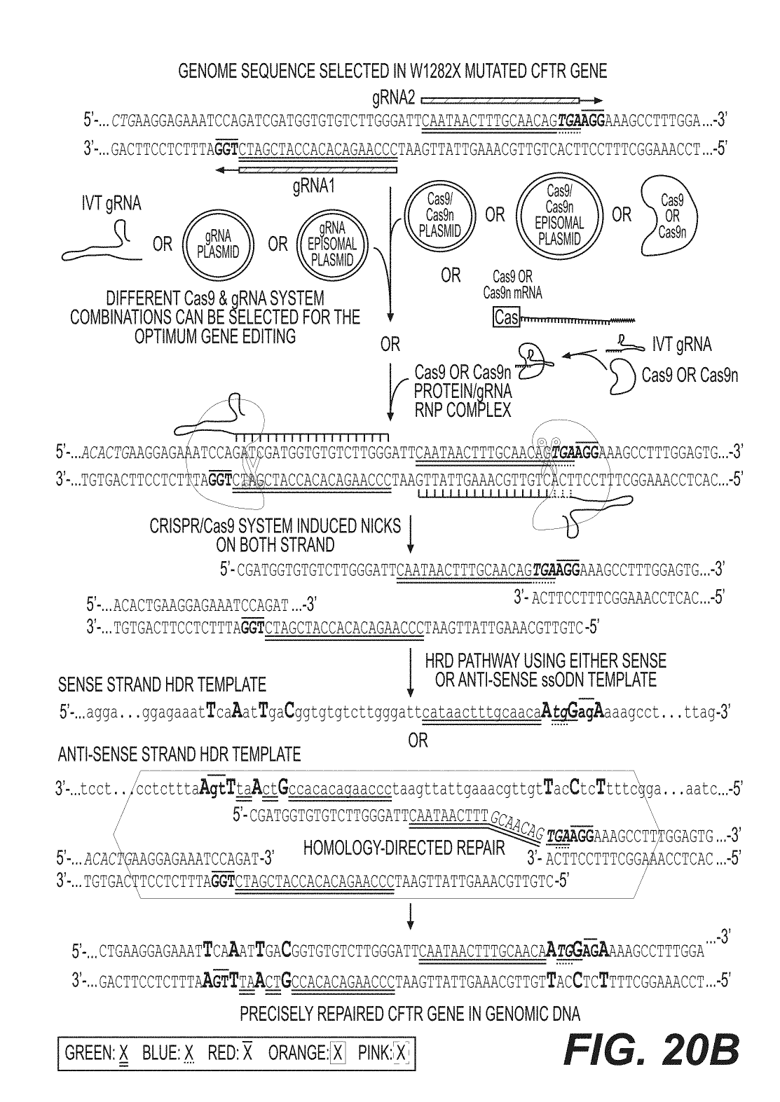

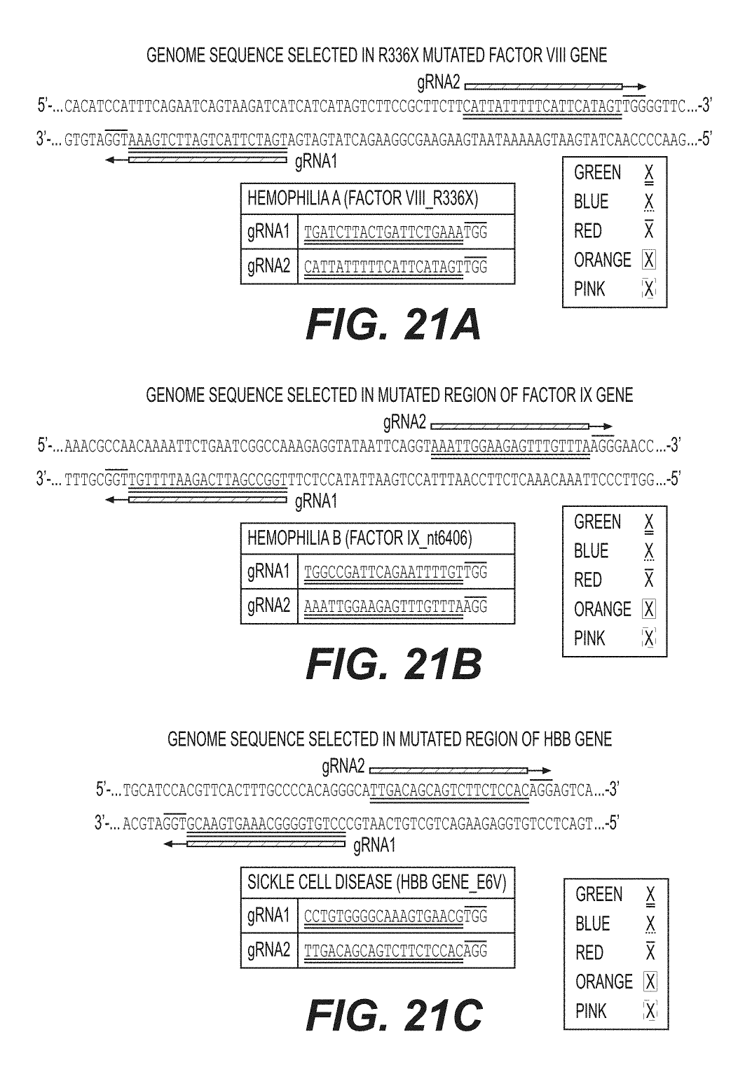

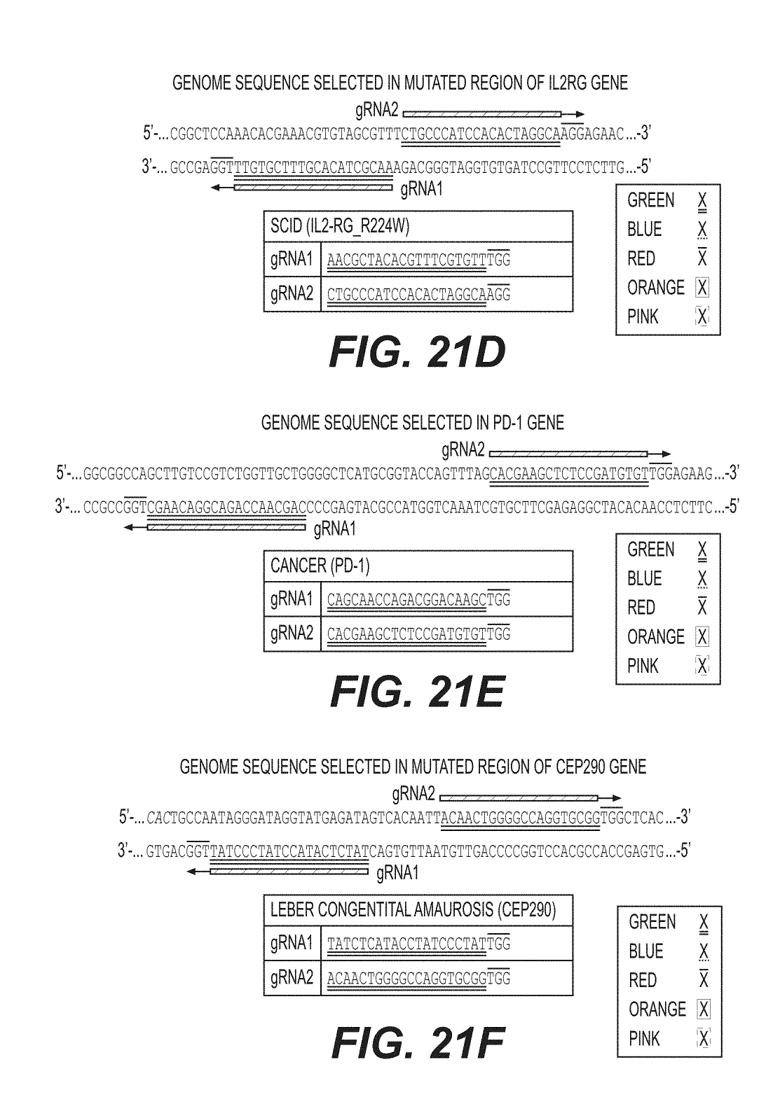

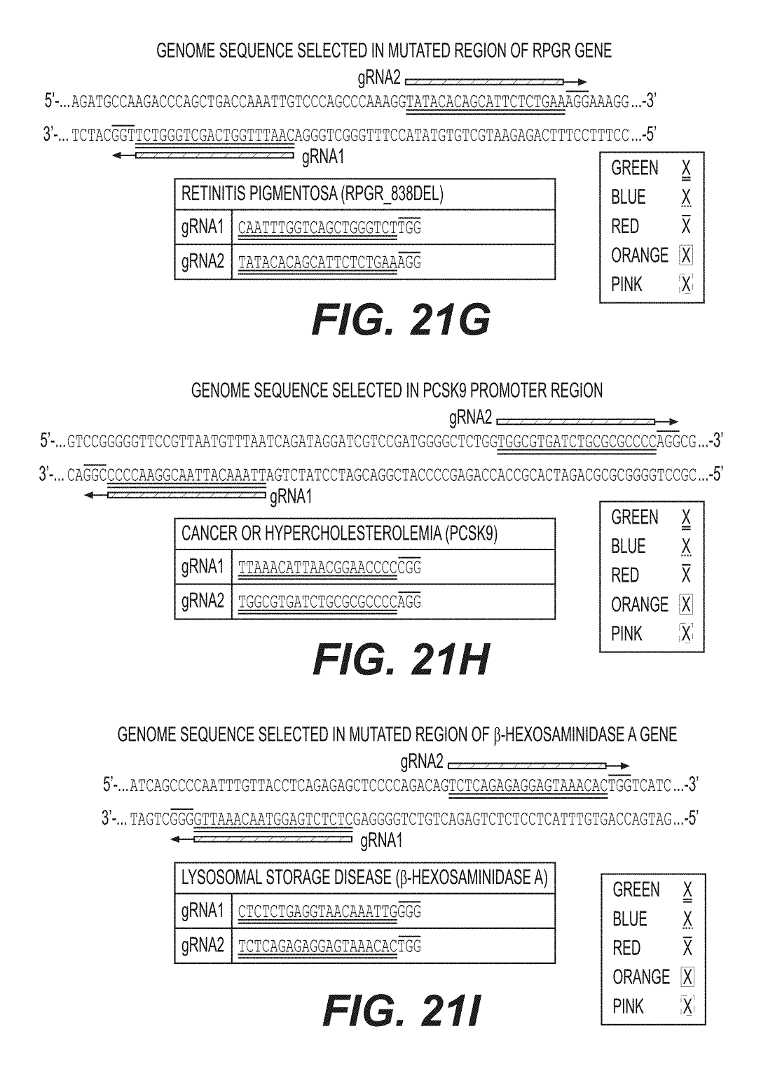

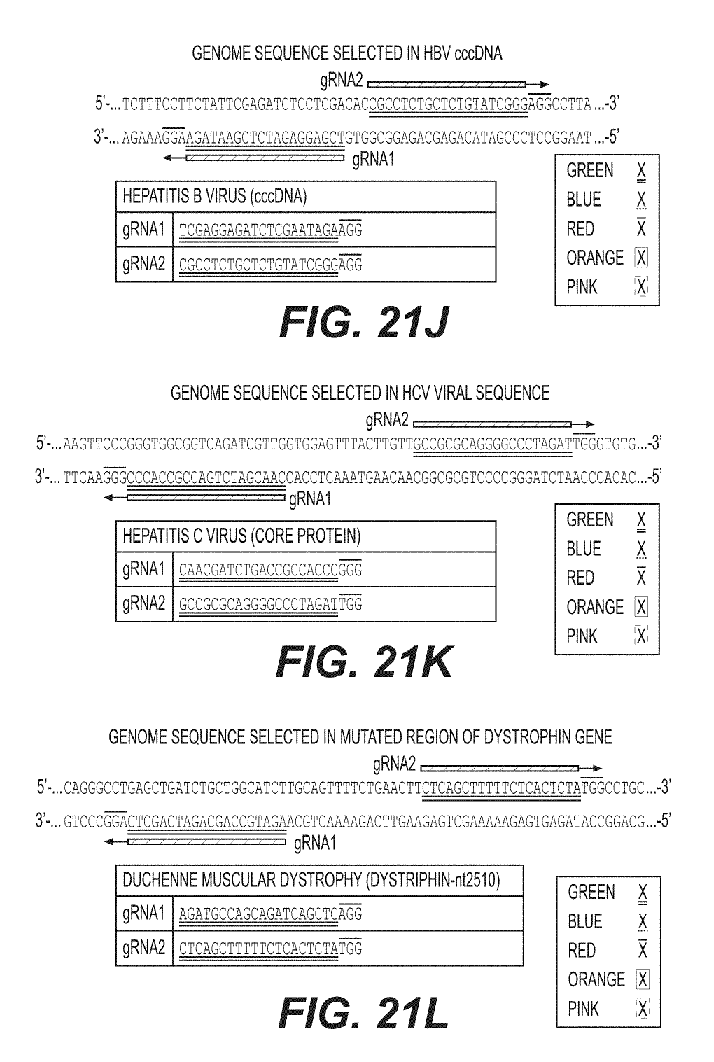

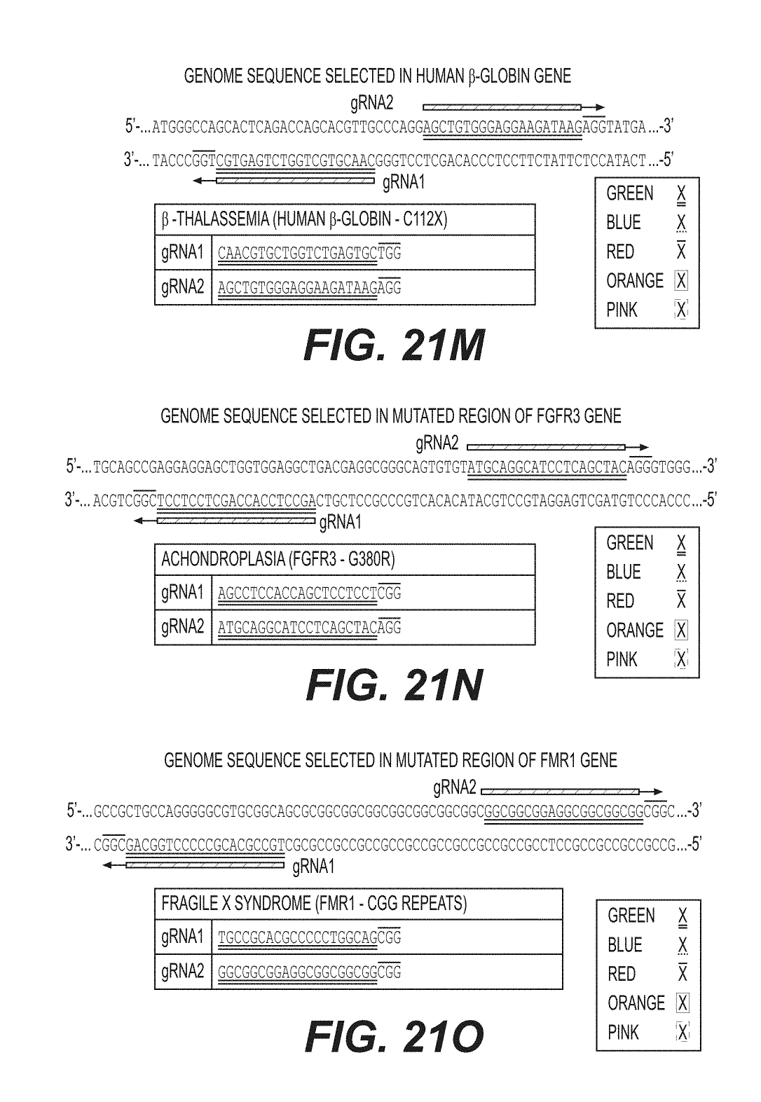

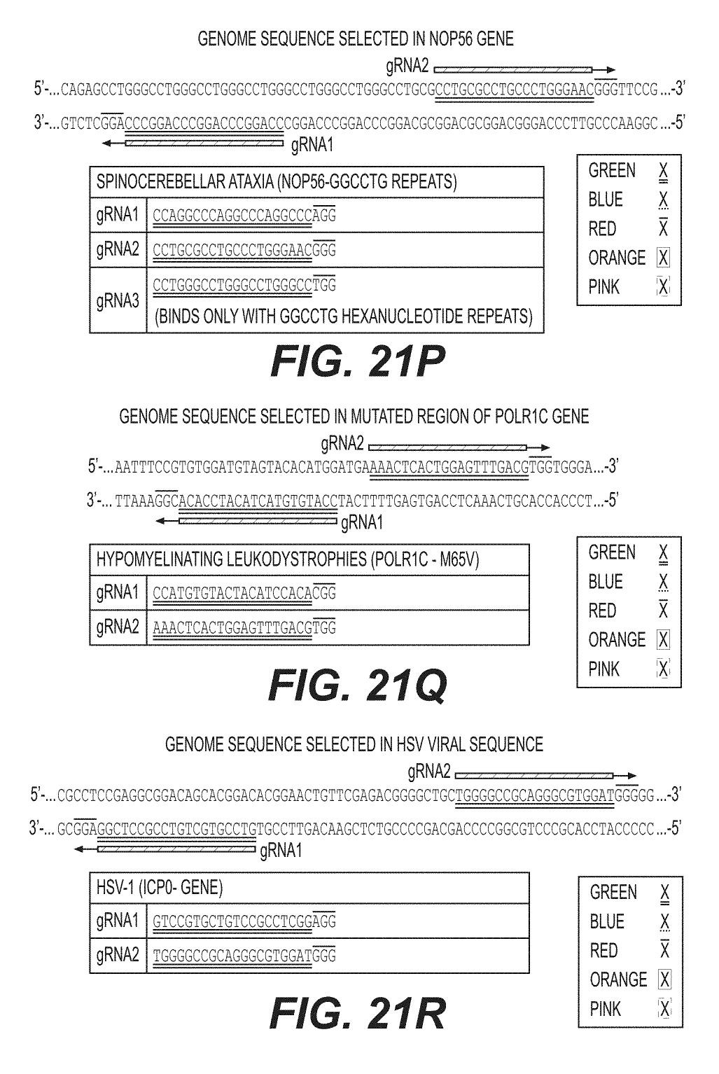

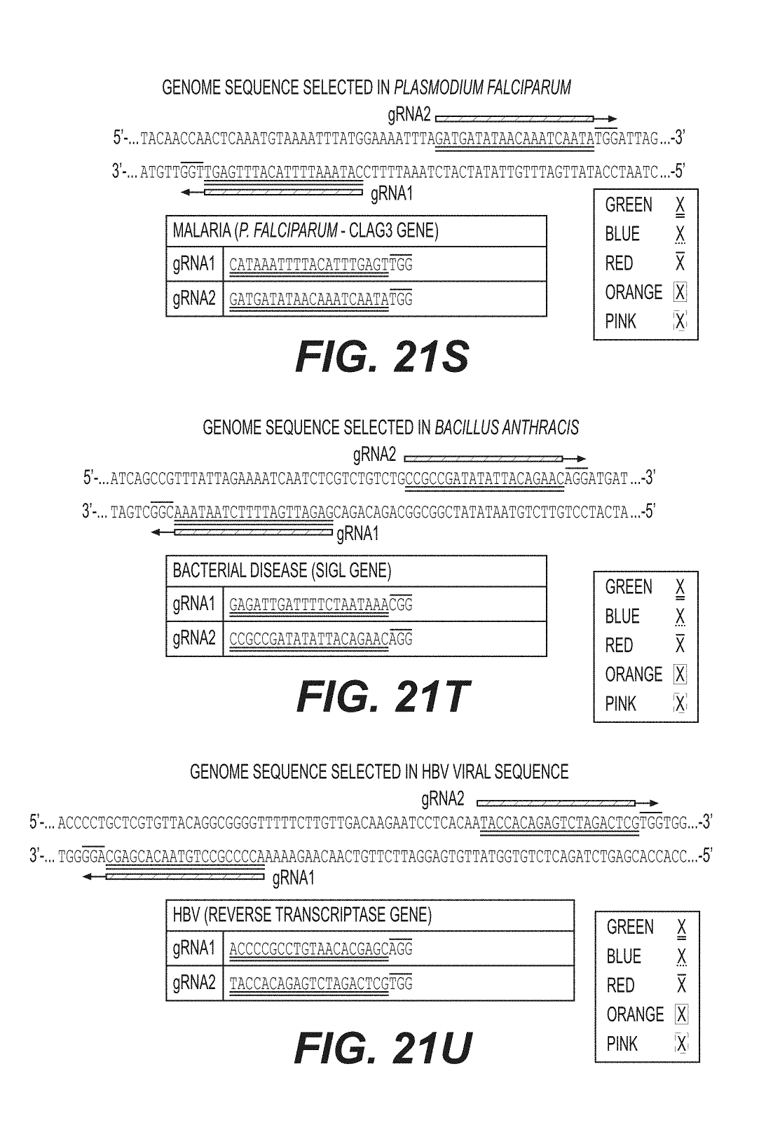

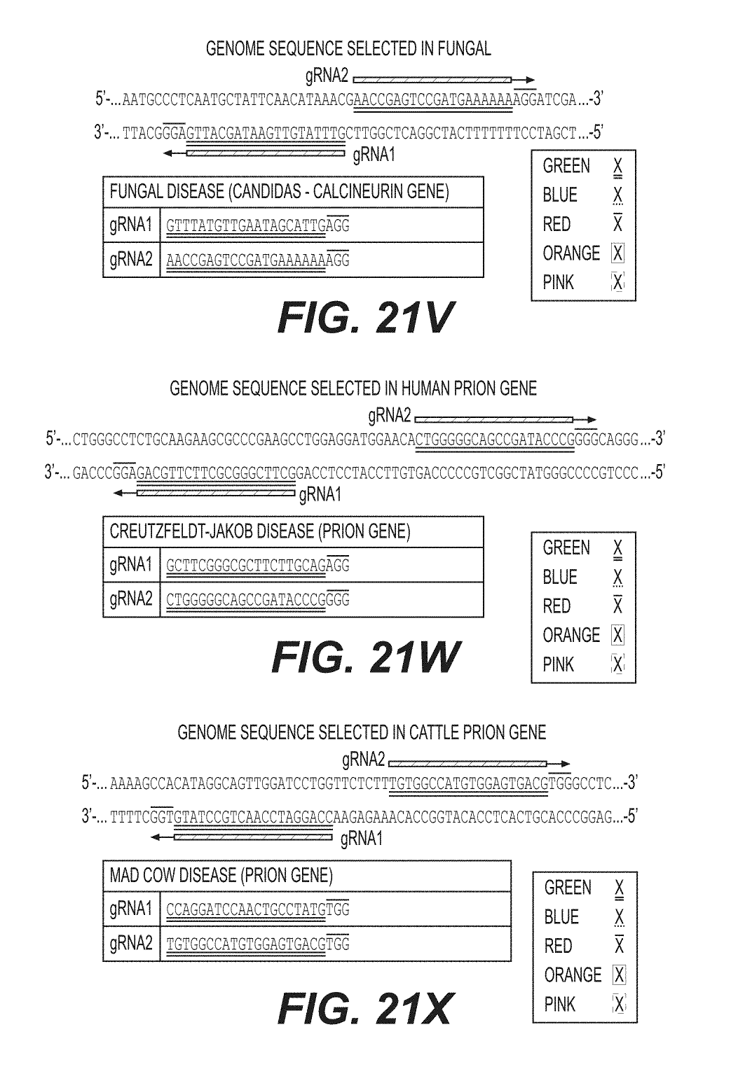

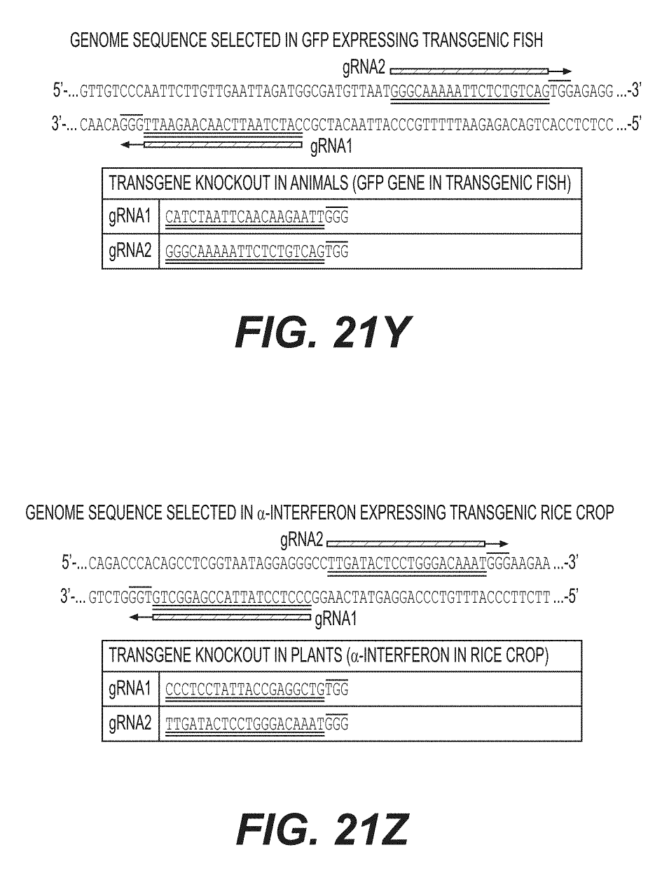

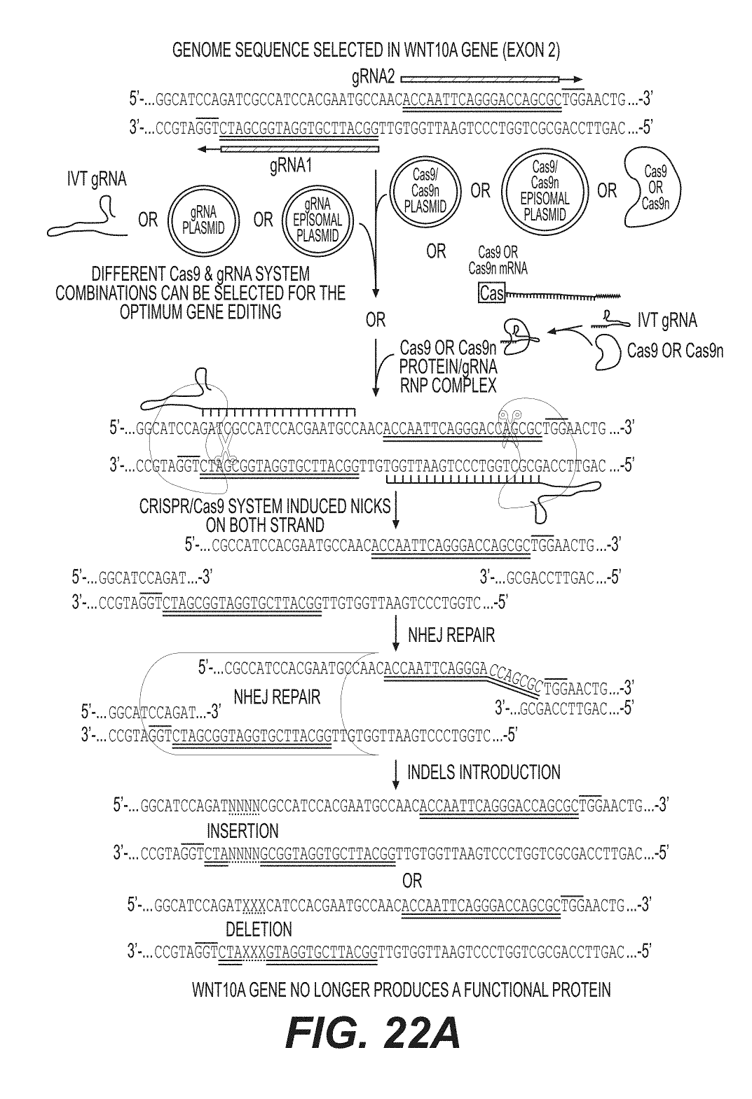

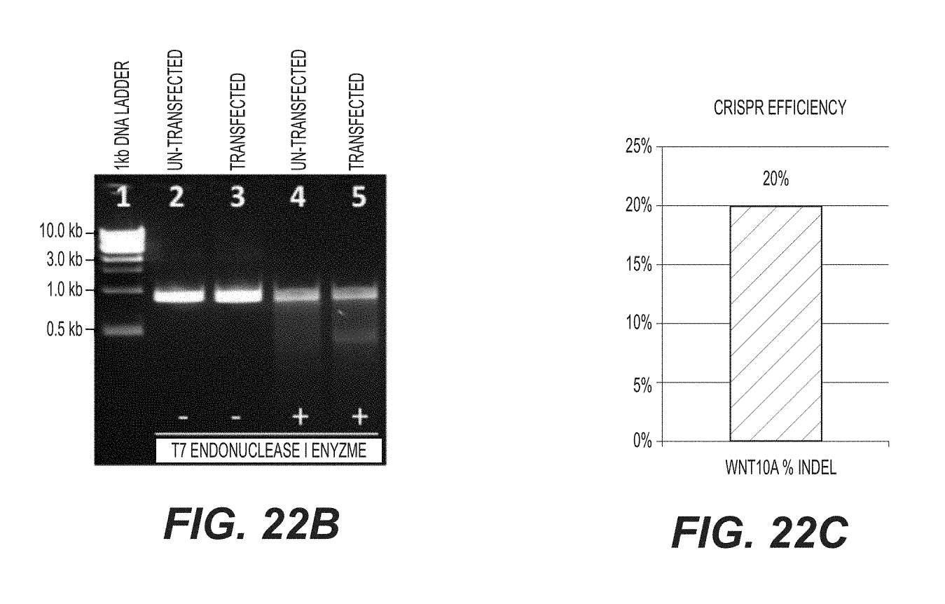

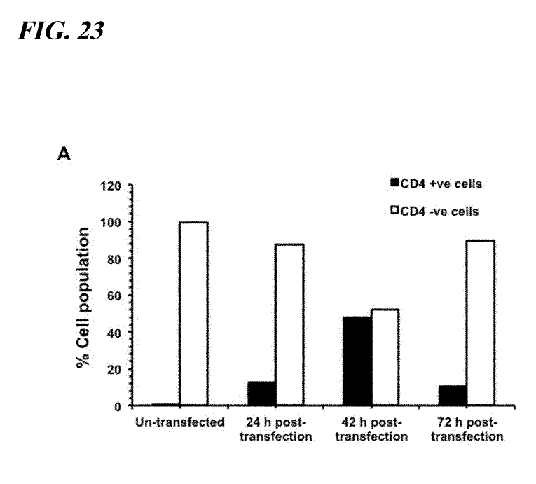

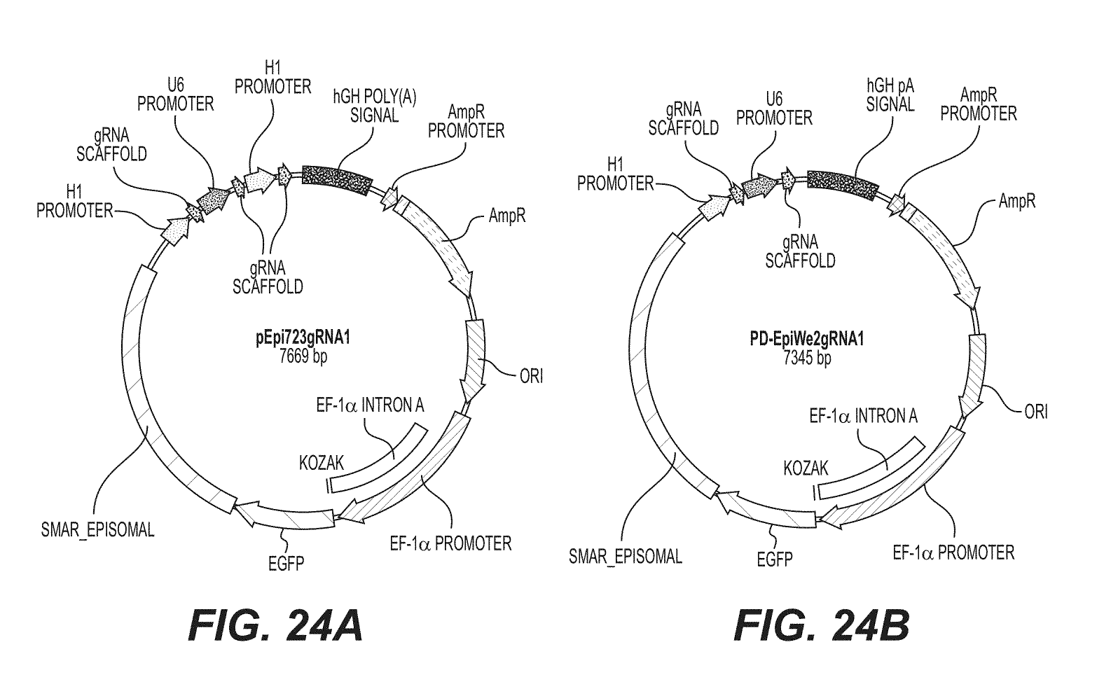

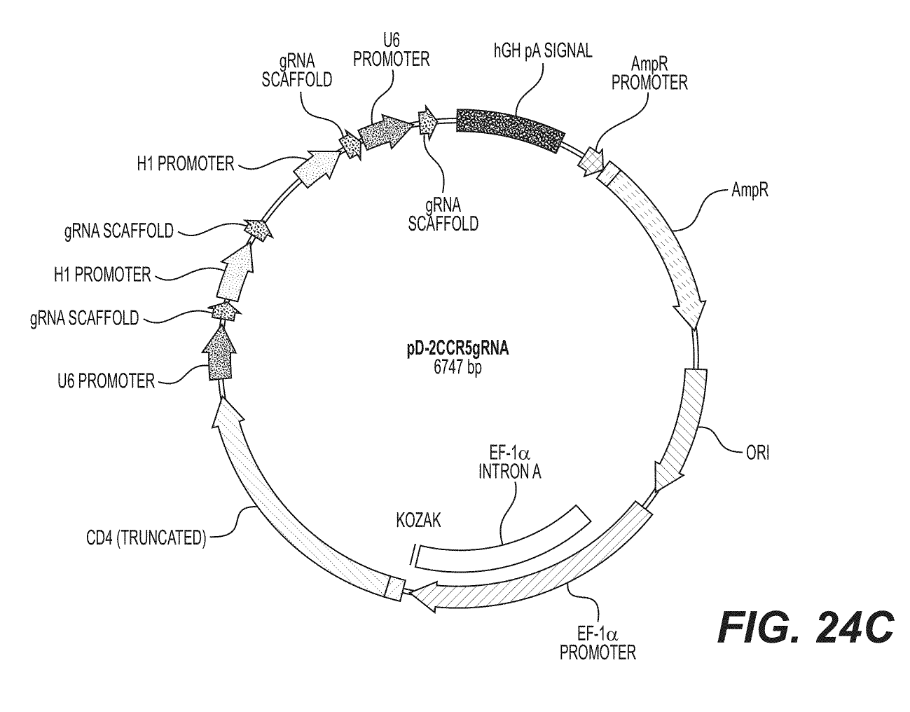

TABLE 6
Examples of gRNAs designed to target genetic mutations in various diseases
or to inactivate transgenes in genetically-modified organisms.

| | | | | | SEQ | |
|---|---|---|---|---|---|---|
| | | | | | ID | Reference |
| | Disease | Gene | NHEJ/HDR | gRNAs¹ | NO: | Figure |
| 1 | Hemophilia A | Factor VIII | HDR | gRNA1: | 12 | FIG. 21A |
| | | | | TGATCTTACTGATTCTGAAA [symbol] | | |
| | | | | gRNA2: | 13 | |
| | | | | CATTATTTTTCATTCATAGT [symbol] | | |
| 2 | Hemophilia B | Factor IX | HDR | gRNA1: | 14 | FIG. 21B |
| | | | | TGGCCGATTCAGAATTTTGT [symbol] | | |
| | | | | gRNA2: | 15 | |
| | | | | AAATTGGAAGAGTTTGTTTA [symbol] | | |
| 3 | Sickle Cell | Human β- | HDR | gRNA1: | 16 | FIG. 21C |
| | Disease | globin (HBB) | | CCTGTGGGGCAAAGTGAACG [symbol] | | |
| | | | | gRNA2: | 17 | |
| | | | | TTGACAGCAGTCTTCTCCAC [symbol] | | |
| 4 | SCID | Various genes | HDR | gRNA1: | 18 | FIG. 21D |
| | | Example: | | AACGCTACACGTTTCGTGTT [symbol] | | |
| | | IL2RG | | gRNA2: | 19 | |
| | | | | CTGCCCATCCACACTAGGCA [symbol] | | |
| 5 | Cancer | Various genes | NHEJ | gRNA1: | 20 | FIG. 21E |
| | | Example: PD-1 | | CAGCAACCAGACGGACAAGC [symbol] | | |
| | | gene | | gRNA2: | 21 | |
| | | | | CACGAAGCTCTCCGATGTGT [symbol] | | |
| 6 | Leber Congenital | CEP290 | HDR | gRNA1: | 22 | FIG. 21F |
| | Amaurosis | | | TATCTCATACCTATCCCTAT [symbol] | | |
| | | | | gRNA2: | 23 | |
| | | | | ACAACTGGGGCCAGGTGCGG [symbol] | | |
| 7 | Retinitis | RPGR | HDR | gRNA1: | 24 | FIG. 21G |
| | Pigmentosa | | | CAATTTGGTCAGCTGGGTCT [symbol] | | |
| | | | | gRNA2: | 25 | |
| | | | | TATACACAGCATTCTCTGAA [symbol] | | |
| 8 | Hyper- | PCSK9 | NHEJ | gRNA1: | 26 | FIG. 21H |
| | cholesterolmeia | | | TTAAACATTAACGGAACCCC [symbol] | | |
| | | | | gRNA2: | 27 | |
| | | | | TGGCGTGATCTGCGCGCCCC [symbol] | | |
| 9 | Lysosomal | Various genes | HDR | gRNA1: | 28 | FIG. 21I |
| | storage disease | Example: β- | | CTCTCTGAGGTAACAAATTG [symbol] | | |
| | | hexosaminidase | | gRNA2: | 29 | |
| | | A | | TCTCAGAGAGGAGTAAACAC [symbol] | | |
| 10 | Hepatitis B Virus | Covalently | NHEJ | gRNA1: | 30 | FIG. 21J |
| | | closed circular | | TCGAGGAGATCTCGAATAGA [symbol] | | |
| | | DNA (cccDNA) | | gRNA2: | 31 | |
| | | | | CGCCTCTGCTCTGTATCGGG [symbol] | | |
| 11 | Hepatitis C Virus | Positive sense | NHEJ | gRNA1: | 32 | FIG. 21K |
| | | single- | | CAACGATCTGACCGCCACCC [symbol] | | |
| | | stranded viral | | gRNA2: | 33 | |
| | | RNA (+ssRNA). | | GCCGCGCAGGGGCCCTAGAT [symbol] | | |
| 12 | HIV | Knockout of | HDR | gRNA1: | 34 | FIG. 8A |
| | | HIV co- | | CAGAATTGATACTGACTGTA [symbol] | | |
| | | receptor CCR5 | | gRNA2: | 35 | |
| | | | | AAAGATAGTCATCTTGGGGC [symbol] | | |
| | | Inactivation of | NHEJ | gRNA1: | 36 | FIG. 8E |
| | | integrated HIV | | CCCCTATCTTTATTGTGACG [symbol] | | |
| | | genome | | gRNA2: | 37 | |
| | | | | AAGGAAGCTCTATTAGATAC [symbol] | | |
| 13 | Cystic Fibrosis | CFTR | HDR | gRNA1: | 38 | FIG. 20B |
| | | | | CCCAAGACACACCATCGATC [symbol] | | |
| | | | | gRNA2: | 39 | |
| | | | | CAATAACTTTGCAACAGTGA [symbol] | | |
| 14 | Duchenne | Dystrophin | HDR | gRNA1: | 40 | FIG. 21L |
| | Muscular | | | AGATGCCAGCAGATCAGCTC [symbol] | | |
| | Dystrophy | | | gRNA2: | 41 | |
| | | | | CTCAGCTTTTTCTCACTCTA [symbol] | | |
| 15 | β-thalassemia | Human β- | HDR | gRNA1: | 42 | FIG. 21M |
| | | globin (HBB) | | CAACGTGCTGGTCTGAGTGC [symbol] | | |
| | | | | gRNA2: | 43 | |
| | | | | AGCTGTGGGAGGAAGATAAG [symbol] | | |
| 16 | Achondroplasia | FGFR3 | HDR | gRNA1: | 44 | FIG. 21N |
| | | | | AGCCTCCACCAGCTCCTCCT [symbol] | | |
| | | | | gRNA2: | 45 | |
| | | | | ATGCAGGCATCCTCAGCTAC [symbol] | | |
| 17 | Amyotrophic | SOD1 | NHEJ | gRNA1: | 46 | FIG. 11B |
| | Lateral Sclerosis | (Several | | GCTAGGCCACGCCGAGGTCC [symbol] | | |
| | (ALS) | mutations) | | gRNA2: | 47 | |
| | | | | AGTTATGGCGACGAAGGCCG [symbol] | | |
| | | | HDR | gRNA1: | 48 | FIG. 7A |
| | | | | CATGAACACGGAATCCATGC [symbol] | | |
| | | | | gRNA2: | 49 | |
| | | | | TTGGAGATAATACAGCAGGT [symbol] | | |
| 18 | Amyotrophic | Caused by | Excision of | gRNA1: | 50 | FIG. 6A |
| | Lateral Sclerosis | intronic | extra | GAGTCGCGCGCTAGGGGCCG [symbol] | | |
| | (ALS) | GGGGCC | GGGGCC | gRNA2: | 51 | |
| | | hexanucleotide | hexanucleotide | CCGGGGCCGGGGCCGGGGCG [symbol] | | |
| | | expansion in | expansion by | gRNA3: | 52 | |
| | | C9ORF72 | NHEJ | CCCGGCCCCGGCCCCGGCCC [symbol] | | |
| | | gene | | | | |
| 19 | Fragile X | Caused by | Excision of | gRNA1: | 53 | FIG. 21O |
| | Syndrome | CGG | extra CGG | TGCCGCACGCCCCCTGGCAG [symbol] | | |
| | | trinucleotide | trinucleotide | gRNA2: | 54 | |
| | | expansion in | expansion by | GGCGGCGGAGGCGGCGGCGG [symbol] | | |
| | | 5′-untranslated | NHEJ | | | |
| | | region of | | | | |
| | | FMR1 gene | | | | |
| 20 | SCA36, a type of | Caused by | Excision of | gRNA1: | 55 | FIG. 21P |
| | Spinocerebellar | intronic | extra | CCAGGCCCAGGCCCAGGCCC [symbol] | | |
| | Ataxia | GGCCTG | GGCCTG | gRNA2: | 56 | |
| | | hexanucleotide | intronic | CCTGCGCCTGCCCTGGGAAC [symbol] | | |
| | | expansion in | hexanucleotide | gRNA3: | 57 | |
| | | NOP56 gene | expansion by | CCTGGGCCTGGGCCTGGGCC [symbol] | | |
| | | | NHEJ | | | |
| 21 | Hypomyelinating | POLR1C | HDR | gRNA1: | 58 | FIG. 21Q |
| | Leukodystrophies | | | CCATGTGTACTACATCCACA [symbol] | | |
| | (HLD) | | | gRNA2: | 59 | |
| | | | | AAACTCACTGGAGTTTGACG [symbol] | | |
| 22 | MELAS | Mitochondrial | HDR | gRNA1: | 60 | FIG. 10B |
| | (mitochondrial | DNA | | AGCGGTAACTAAGATTAGTA [symbol] | | |
| | encephalomyopathy, | | | gRNA2: | 61 | |
| | lactic acidosis, and | | | TTCCAACTGTTCATCGGCTG [symbol] | | |
| | stroke-like episodes) | | | | | |
| 23 | Virus affecting | ICP0 | NHEJ | gRNA1: | 62 | FIG. 21R |
| | neurons (Ex. | | | GTCCGTGCTGTCCGCTCGG [symbol] | | |
| | Herpes virus) | | | gRNA2: | 63 | |
| | | | | TGGGGCCGCAGGGCGTGGAT [symbol] | | |
| 24 | Human parasite: | Clag3 | NHEJ | gRNA1: | 64 | FIG. 21S |
| | Plasmodium | | | CATAAATTTTACATTTGAGT [symbol] | | |
| | falciparum | | | gRNA2: | 65 | |
| | | | | GATGATATAACAAATCAATA [symbol] | | |
| 25 | Human Bacterial | Sigl | NHEJ | gRNA1: | 66 | FIG. 21T |
| | disease: Anthrax | | | GAGATTGATTTTCTAATAAA [symbol] | | |
| | | | | gRNA2: | 67 | |
| | | | | CCGCCGATATATTACAGAAC [symbol] | | |
| 26 | Human viral | Reverse | NHEJ | gRNA1: | 68 | FIG. 21U |
| | disease: HBV | Transcriptase | | ACCCCGCCTGTAACACGAGC [symbol] | | |
| | | | | gRNA2: | | |
| | | | | TACCACAGAGTCTAGACTCG [symbol] | 69 | |
| 27 | Human | Calcineurin | NHEJ | gRNA1: | 70 | FIG. 21V |
| | Fungal/Yeast | | | GTTTATGTTGAATAGCATTG [symbol] | | |
| | disease: | | | gRNA2: | 71 | |
| | candidiasis | | | AACCGAGTCCGATGAAAAAA [symbol] | | |
| 28 | Prion Disease: | Human Prion | NHEJ | gRNA1: | 72 | FIG. 21W |
| | Creutzfeldt-Jakob | protein | | GCTTCGGGCGCTTCTTGCAG [symbol] | | |
| | disease | | | gRNA2: | | |
| | | | | CTGGGGGCAGCCGATACCCG [symbol] | 73 | |
| 29 | Prion Disease: | Cattle Prion | NHEJ | gRNA1: | 74 | FIG. 21X |
| | Mad Cow disease | protein | | CCAGGATCCAACTGCCTATG [symbol] | | |
| | | | | gRNA2: | 75 | |
| | | | | TGTGGCCATGTGGAGTGACG [symbol] | | |
| 30 | Knockout of | GFP | NHEJ | gRNA1: | 76 | FIG. 21Y |
| | previously inserted | expressing | | CATCTAATTCAACAAGAATT [symbol] | | |
| | transgene in | transgenic fish | | gRNA2: | 77 | |
| | species | | | GGGCAAAAATTCTCTGTCAG [symbol] | | |
| | | α-interferon | NHEJ | gRNA1: | 78 | FIG. 21Z |
| | | expressing | | CCCTCCTATTACCGAGGCTG [symbol] | | |
| | | transgenic rice | | gRNA2: | 79 | |
| | | crop | | TTGATACTCCTGGGACAAAT [symbol] | | |

¹Sequences are shown in the 5′ → 3′ direction. PAM sequences are shown in bold and underlined.

### Example 8. Repeated Correction of Non-Functional Genes to Increase the Incidence of HDR

HDR is generally a very precise DNA repair pathway compared to NHEJ, however, NHEJ is generally more active than HDR-mediated DNA repair. This difference in efficiency makes HDR-mediated gene editing more challenging for correcting or editing genes, as well as for use in disease treatment or gene enhancement strategies that require precise gene editing or correction, as compared to gene inactivation by NHEJ. To overcome this difference in efficiency, we have developed an HDR-mediated gene-editing strategy with multiple transfections of HDR-plasmid and a corrective HDR donor in order to target disease-causing mutations or for gene enhancement.

In order to illustrate such methods, an example is shown in FIG. 20, which details an HDR therapeutic strategy for treating cystic fibrosis caused by the W1282X mutation (an example of a nonsense or premature termination mutation). FIG. 20 shows the design of the two gRNAs and ssODN for correcting the W1282X mutation. The method includes several rounds of introducing the HDR-plasmid and the corrective HDR template that selectively targets the W1282X mutation to a population of cells having the W1282X mutation, in order to correct the premature termination codon (PTC) and reintroduce the tryptophan (W) codon. During this process, indels may form in several cells along with HDR-mediated repair in other cells. In cases of less than 100% efficiency of introducing the HDR plasmid and corrective HDR donor, some cells will not receive the HDR plasmid, while other cells will not receive the corrective HDR donor, or both, during any one round of introduction (e.g., by transfection). As the PTC may have already resulted in completely non-functional protein synthesis, there will be no safety concern if an indel mutation is present, as the gene is already completely non-functional. Every attempt to introduce the HDR-plasmid and the corrective HDR template into the cells will significantly increase the number of corrected cells, as the ssODN and gRNA have been designed not to modify already-corrected cells from previous rounds. The system thus allows for efficient and safe restoration of protein expression using HDR-mediated genome editing. It is noted that there are hundreds of mutations that cause PTC and result in synthesis of non-functional proteins (Atkinson, J. and Martin, R., Nucleic Acids Res. 22(8):1327-34, 1994). The same or similar methods can be applied to treat similar diseases caused by non-functional protein synthesis.

### Example 9. Therapeutic Applications of Indel-Mediated Genome Editing Against Cancer

Cancer is a group of diseases involving uncontrolled proliferation of abnormal cells in the body with the potential to invade or spread to other parts of the body. Current cancer treatment strategies generally target all the dividing cells instead of specifically targeting the abnormally proliferating cancer cells. Targeted cancer therapies are expected to be more efficient than older treatment forms and less harmful to the healthy cells. The methods detailed herein allow for a gene editing approach using the cellular NHEJ pathway to knockout oncogenes that disrupt the normal cell cycle (though overexpressing their encoded oncoproteins, leading to cancer development). This CRISPR/Cas9 system mediated gene editing involves the introduction of Cas9n protein (either via a plasmid, mRNA, or protein) and gRNA into cells to correct the cellular dysfunction or to knockout certain genes within the cells to cure or slow cancer progression. Recently, many genes and associated pathways have been identified as being dysregulated in cancer. Examples of such genes are shown in Table 7. Gene editing provides a rationale towards specifically targeting such genes to cure the underlying cancer diseases. As an example, WNT10A acts as an autocrine oncogene both in renal cell carcinogenesis and in its progression by activating the WNT/β-catenin signaling cascade (Hsu et al., 2012 PLOS one 7(10): e47649).

TABLE 7
Examples of oncogene mutations and associated pathways that cause cancer.
(Reference: Futreal, P. A. et al., Nat. Rev. Cancer 4: 177-183, 2004.)

| Gene | | Entrez | Tumour Types | Tumour Types |
|---|---|---|---|---|
| Symbol | Name | Gene Id | (Somatic) | (Germline) |
| BMPR1A | Bone Morphogenetic | 657 | | Gastrointestinal |
| | Protein Receptor | | | polyps |
| | Type 1A | | | |
| CDK4 | Cyclin-dependent | 1019 | | Melanoma |
| | kinase 4 | | | |
| ERBB2 | Erb-B2 Receptor | 2064 | Breast, ovarian, NSCLC, | |
| | Tyrosine Kinase 2 | | gastric | |
| ERBB3 | Erb-b2 receptor | 2065 | Colon, gastric, head and | |
| | tyrosine kinase 3 | | neck, bladder, skin | |
| FH | Fumarate hydratase | 2271 | | Leiomyomatosis, |
| | | | | renal |
| FLCN | Folliculin | 201163 | | Renal, |
| | | | | fibrofolliculomas, |
| | | | | trichodiscomas |
| HIF1A | Hypoxia inducible | 3091 | Glioblastoma, colorectal, | |
| | factor 1 alpha subunit | | renal, lung, pancreatic | |
| KRAS | KRAS Proto- | 3845 | Pancreatic, colorectal, lung, | |
| | Oncogene, GTPase | | thyroid, AML | |
| MDM2 | MDM2 Proto- | 4193 | Sarcoma, glioma, | |
| | Oncogene | | colorectal, other tumour | |
| | | | types | |
| MET | Hepatocyte growth | 4233 | Papillary renal, head-neck | Papillary renal |
| | factor receptor | | squamous cell | |
| MLH1 | MutL homolog gene | 4292 | Colorectal, endometrial, | Colorectal, |
| | | | ovarian, CNS tumours | endometrial, |
| | | | | ovarian, CNS |
| MSH2 | MutS homolog 2 | 4436 | Colorectal, endometrial, | Colorectal, |
| | | | ovarian | endometrial, |
| | | | | ovarian |
| MSH6 | MutS homolog 6 | 2956 | Colorectal | Colorectal, |
| | | | | endometrial, |
| | | | | ovarian |
| MYC | Proto-Oncogene C- | 4609 | Burkitt lymphoma, | |
| | Myc | | amplified in other cancers, | |
| | | | B-CLL | |
| PMS1 | Postmeiotic | 5378 | | Colorectal, |
| | segregation | | | endometrial, |
| | increased 1 | | | ovarian |
| PMS2 | Postmeiotic | 5395 | | Colorectal, |
| | segregation | | | endometrial, |
| | increased 2 | | | ovarian, |
| | | | | medulloblastoma, |
| | | | | glioma |
| REL | Proto-Oncogene C- | 5966 | Hodgkin lymphoma | |
| | Rel | | | |
| RET | Ret Proto-Oncogene | 5979 | Medullary thyroid, | Medullary thyroid, |
| | | | papillary thyroid, | papillary thyroid, |
| | | | pheochromocytoma, | pheochromocytoma |
| | | | NSCLC, Spitzoid tumour | |
| SRC | SRC Proto- | 6714 | Colorectal cancer, | |
| | Oncogene, Non- | | endometrial carcinoma | |
| | Receptor Tyrosine | | | |
| | Kinase | | | |
| STAT3 | Signal transducer and | 6774 | T-cell large granular | Paediatric large |
| | activator of | | lymphocytic leukaemia | granular |
| | transcription 3 | | | lymphocytic |
| | | | | leukaemia |
| WNT10A | Wnt Family Member | 80326 | | Renal cell |
| | 10A | | | carcinogenesis |

WNT10A gene knockout in Caki-1 cells was used here as an example to demonstrate a quantitative platform to directly test the performance of the knockout of certain oncogenes for inhibiting carcinogenesis and its disease progression. Caki-1 cells (#HTB-46, ATCC) were cultured in McCoy's 5A medium (#SH3020001, Hyclone) supplemented with 10% bovine calf serum (#SH30073.04, Hyclone) at 37° C., 5% CO₂. 1×10⁶ cells were harvested using TrypLE Select (#A1285901, Thermofisher), and the harvested cells were re-suspended in 1×PBS and spun down at 200 g for 3 minutes. The pelleted cells were gently re-suspended in 100 μl of SF solution (PBC2-00675, Lonza) and transfected with 1 μg of pD-EpiWe2gRNA1 plasmid (SEQ ID NO: 107) and 1 μg of Cas9n mRNA (SEQ ID NO: 109). The cell suspension was combined with the mix of plasmid DNA and mRNA, and transferred to a nucleofection cuvette and transfected using “DN-100” program in a 4D-Amaxa Nucleofector™ device (Lonza). After transfection, 100 μl of the medium was mixed with the cell suspension in the cuvette and incubated for 10 min at 37° C., 5% CO₂. The cells were then transferred to 2 wells of a 6-well plate (#3335, Costar) and incubated at 37° C., 5% CO₂. After 72 hours, the cells were harvested with TrypLE Select and plated out over 2 new wells of a 6-well plate. The next day, the cells were transfected with additional Cas9n mRNA. The RNA transfection mix was prepared using MessengerMax (#LMRNA008, Thermofisher) as per the manufacturer's protocol: About 15 μl of MessengerMax was diluted in 250 μl OptiMEM I and incubated for 10 minutes at room temperature. About 5 μg of Cas9n mRNA was diluted in 250 μl OptiMEM I media and mixed with the MessengerMax solution and incubated for 5 minutes at room temperature. After 5 minutes of incubation, 250 μl of the transfection mix were then added to each well containing 2.5 mL medium and 0.5 mL DNA transfection mix. The cells were incubated with this transfection mix for 4-6 h at 37° C., 5% CO₂, The media was then replaced with 3 mL of fresh medium per well, and the cells were incubated at 37° C., 5% CO₂. This Cas9n mRNA transfection was repeated two more times 72 h apart. 4 days after the final transfection the cells were harvested for the analysis of CRISPR/Cas9 mediated cleavage efficiency using T7 endonuclease mutation detection assay. The genomic DNA was isolated from CRISPR/Cas9 system transfected cell lines using Quick-gDNA Miniprep Kit (# D3024, Zymo Research). PCRs were performed using Phusion™ High-Fidelity PCR Master Mix (ThermoFisher, MA USA). The following primers were used to amplify the gDNA region containing the CRISPR target site: The forward primer NWL-MBPr-1081 (5′-atactgtggccacaagcatg-3′)(SEQ ID NO: 110) and reverse primer NWL-MBPr-1080 (5′-gttccccatcctaaatgtgg-3′)(SEQ ID NO: 111) were used to amplify a 898-bp product from both edited and untransfected Caki-1 cells, with the cleavage site located approximately in the middle. The PCR cycling parameters used with a C1000 Thermal Cycler (Bio-Rad, CA, USA) were as follows: initial denaturation at 98° C. for 3 min, 32 cycles of 98° C. for 30 seconds, 60° C. for 30 seconds, 72° C. for 60 seconds and the final extension at 72° C. for 5 min. After amplification, PCR products were resolved by 1.5% agarose gel electrophoresis and the amplicon purified using Wizard SV gel and PCR cleanup system (#A9281, Promega). Approximately 300 ng of purified PCR product obtained from the untransfected and edited cells were denatured at 95° C. and re-annealed in NEB buffer 2 using C1000 Thermal Cycler (Bio-Rad, CA, USA) as follows: 95° C. 5 min, ramp down to 85° C. at 2° C./second, followed by ramp down to 25° C. at 0.1° C./second and final hold at 4° C. The re-annealed PCR products were digested with 10 U of T7 endonuclease I (#M0302L, NEB) for 30 min at 37° C. The reaction was stopped by adding 2 μl of 0.5 M EDTA, and PCR products were resolved by electrophoresis on a 2% agarose gel. DNA fragments were stained with SYBR safe (#S33102, Invitrogen) according to manufacturer's procedure, and ImageJ software was used for the quantification of band intensities and gene editing efficiency. The gene editing efficiency was determined to be 20.1%. This efficiency was obtained without optimizing any of the transfection conditions or quantity and stability of the Cas9n mRNA introduced into the cells, and without any selection of the cells. Targeting efficiencies were calculated using the following formula: % gene modification=100×(1−[1−fraction cleaved]^{1/2}). The fraction cleaved was calculated using the following formula: Cleaved band intensity/(uncleaved band intensity+cleaved band intensity).

### Example 10. Selection of Transfected Cells Using Truncated Proteins with or without Tag-Epitopes, for Enrichment of CRISPR/Cas9-Mediated Genome-Edited Cells

Without wishing to be limited by theory, one advantage of using a non-integrating episomal plasmid is that the gRNA and/or Cas9 can be introduced continuously over a sufficient period of time, to help ensure that the gene editing takes place in the cell, thereby increasing efficiency. For example, an episomal plasmid encoding the one or more gRNA can help to ensure that sufficient gRNA is continuously present to allow for multiple introductions of Cas9 (either as the protein or as mRNA or a plasmid) and for precise timing (e.g., at a particular point in the cell cycle or when a donor template/ssDNA is introduced into the cell). This can provide an overall higher efficiency of achieving gene editing in a high proportion of cells in a cell population. Alternatively, an episomal plasmid can encode Cas9 to ensure continuous presence of the Cas9 protein in the cell over a prolonged period of time; the one or more gRNA can then be introduced (optionally together with the donor template) multiple times to allow high efficiency gene editing in a cell population.

In addition, an episomal plasmid encoding the one or more gRNA and/or Cas9 can also encode for a truncated surface protein or a protein that confers specific antibiotic resistance to a cell, to allow for selection and purification of transfected cells carrying the episomal plasmid (e.g., by sorting or by magnetic antibody separation, or using a specific antibody for the truncated surface protein that is expressed). Thus cells having the episomal plasmid can be selected and purified out of the starting cell population; it could then be expected that all or nearly all of the cells in this selected cell population (which in most cases would represent >50% of the starting cell population) would be gene edited, allowing for a completely pure or nearly pure gene-edited cell population, and without any gene integration (except for the optional donor template).

For antibiotic-free selection, a diverse pool of non-immunogenic N- or C-terminal truncated proteins with or without tag-epitopes can be used to enrich the transfected cells using sorting, magnetic microbead-based separation, or other separation methodologies (see, e.g., Table 8 and FIG. 4, which provide non-limiting examples of N- or C-terminal truncated proteins and tag-epitopes that may be used). This approach can be utilized for any cell type, whether cells are adherent or grow in suspension, for rapid antibiotic-free selection of transfected cells, in order to enrich the percentage of gene-edited cells. It is noted that in some cases, certain cells may endogenously express all or a few of the above-mentioned truncated proteins; such endogenous expression would interfere with the selection of transfected cells. In such cases, the tag-epitopes can be used as a selection and tracking tool for antibiotic-free selection of transfected cells. Tag-epitopes can be inserted, for example, between the ends of an outer membrane signal peptide and before the start codon of a truncated protein.

Transfection Conditions and CD4 Magnetic Sorting for KG-1 Cells.

CCR5 gene knockout and CD4 truncated protein expression in KG-1 cells was used as an example to demonstrate a quantitative platform to directly test the performance of the truncated protein efficiency in the enrichment of Cas9n mRNA/gRNA plasmid transfected cells and the CD4 truncated protein expression (analyzed using Amnis® FlowSight, a multicolour spectral imaging flow cytometer). KG-1 cells (ATCC CCL-246) were cultured in Iscove's Modified Dulbecco's medium (Thermofisher 12440-053) supplemented with 20% bovine calf serum (Hyclone SH30073.04) at 37° C., 5% CO₂, 5% 0₂ 2×10⁶ cells were centrifuged at 200 g for 2 minutes and re-suspended in PBS and spun down at 200 g for 3 minutes. The cells were gently re-suspended in 100 μL of SF solution (Lonza PBC2-00675) and transfected with 5 μg pD-CCR5gRNA (SEQ ID NO: 108) and 2.5 μg Cas9n mRNA. The cell suspension was combined with the mix of plasmid DNA and mRNA, transferred to a cuvette and transfected with “FF-100” program in a 4D-Amaxa Nucleofector™ Device (Lonza). Post transfection, 100 μL of medium was mixed with the cell suspension in the cuvette and incubated for 10 min at 37° C., 5% CO₂, 5% O₂. The cells were then transferred to a well of a 6-well plate (Costar 3335) and incubated at 37° C., 5% CO₂, 5% O₂ until analysis.

Flow Cytometry and Analysis.

Multicolor spectral imaging flow cytometry data were collected on an Amnis® FlowSight at 20× magnification. For the truncated CD4 expression analysis, the fresh cell pellets from the transfection conditions were tested to determine the relative differences in the expression of truncated CD4 receptors at different time points following transfections with truncated CD4 expression plasmid. Briefly, the fresh cell pellets were first re-suspended into single cell suspension in BD Staining buffer (BSA, #554657, BD Biosciences), and the cells were stained with FITC conjugated anti-CD4 antibody (M-T466, 130-080-501, Miltenyi Biotec) for 15 minutes before rinsing off the unbound antibodies. The stained cells were fixed with BD CytoFix™ Fixation buffer (#554655, BD Biosciences) and rinsed 3× times with 1×PBS. The cells were then re-suspended in 200 μL of 1×PBS, and the truncated CD4 receptor expression was detected using the single color antibody staining. This single color antibody panel was excited with a 488-nm laser at 60 mW (for detecting FITC), and the brightfield and fluorescent images were collected for 50,000 events. The Amnis IDEAS 6.1 software was used to analyze raw image files. The cut-offs for in-focus and single cells were determined manually, and pictures were screened to remove cells that were debris. The relative expression was determined using Frequency Vs Intensity values for FITC fluorophore, and its geometric mean of the histoplots were used to determine the magnitude of relative differences of truncated CD4 expression following transfection with truncated CD4 expression plasmid. From the images and histoplots obtained for different time points following transfection with the CRISPR/Cas9n system, the observed peak of truncated CD4 expression in KG1 cells transfected with the 5 μg pD-CCR5gRNA and 2.5 μg Cas9n mRNA was found to be around 42 hour post transfection, at which point almost half the transfected cells expressed it. In cases where an episomal plasmid was used instead, the truncated CD4 expression could be maintained for several weeks. This approach can thus be used to isolate and purify likely cells that have undergone gene editing, without the need for stable transfection and/or antibiotic selection.

TABLE 8
Examples of truncated proteins and tag-epitopes that can express in
the outer membrane of cells for use in selecting transfected cells.

| N- or C- terminal Truncated Proteins | | |
|---|---|---|
| Truncated proteins | Cell Organelle | Tag-Epitopes |
| CD4 (Cluster of | T helper cells, | 1. 6x Histidine (His) |
| differentiation 4) | monocytes, | 2. c-myc (myc) |
| | macrophages, | 3. Hemagglutinin A (HA) |
| | and dendritic cells | 4. Thioredoxin (TR) |
| H-2K (H-2Kk MHC | All nucleated cells | |
| class I alloantigen) | | |
| LNGFR (Low-affinity | Overexpressed | |
| nerve growth factor | in Nerve | |
| receptor) | | |
| EGFR (Epidermal | Placenta | |
| growth factor receptor) | | |
| VDAC (Voltage- | Mitochondria | |
| dependent anion- | | |
| selective channel) | | |

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

What is claimed is:

1. A method for targeted genomic modification within a target genome region (TGR) in a mammalian cell, the method comprising:
a) providing a CRISPR/Cas9 system comprising:
i) a first guide RNA (gRNA) comprising a first CRISPR RNA (crRNA) and a first trans-activating crRNA (tracrRNA) linked together, the first gRNA being capable of binding with sequence specificity to a first target DNA sequence on one strand of the DNA double helix in the TGR, the first target DNA sequence to which the first gRNA binds being adjacent to a first PAM sequence;
ii) a second gRNA comprising a second CRISPR RNA (crRNA) and a second trans-activating crRNA (tracrRNA) linked together, the second gRNA being capable of binding with sequence specificity to a second target DNA sequence, the second target DNA sequence to which the second gRNA binds being adjacent to a second PAM sequence, wherein the second target DNA sequence is on the same strand of the DNA double helix as the first target DNA sequence;
iii) a third gRNA comprising a third CRISPR RNA (crRNA) and a third trans-activating crRNA (tracrRNA) linked together, the third gRNA being capable of binding with sequence specificity to a third DNA sequence on one strand of the DNA double helix in the TGR, the third target DNA sequence to which the third gRNA binds being adjacent to a third PAM sequence, wherein the third target DNA sequence is on the opposite strand of the DNA double helix from the first and the second target DNA sequences;
wherein the first target DNA sequence and the second target DNA sequence are overlapping, such that the first gRNA and the second gRNA compete for binding to their respective target DNA sequences;
and wherein at least one of the second gRNA and the third gRNA is selected such that the CRISPR/Cas9 system can only bind and/or modify the second target DNA sequence and/or the third target DNA sequence respectively if the target genome region comprises a disease-causing modification or a sequence for which modification is desired; and
iv) a Cas9n protein;
and
b) contacting the mammalian cell with the CRISPR/Cas9 system such that the TGR is modified, forming a modified-TGR.

2. The method of claim 1, wherein the CRISPR/Cas9 system can only bind and/or modify the second and/or the third target DNA sequence in the mammalian cell of a patient suffering from a disease.

3. The method of claim 1 or 2, wherein the third target DNA sequence is only adjacent to the third PAM sequence if the target genome region comprises a disease-causing modification or a sequence for which modification is desired or is in the mammalian cell of a patient suffering from a disease.

4. The method of claim 1 or 2, wherein the second target DNA sequence is only adjacent to the second PAM sequence if the target genome region comprises a disease-causing modification or a sequence for which modification is desired or is in the mammalian cell of a patient suffering from a disease.

5. The method of any one of claims 1 to 4, wherein the second target DNA sequence and/or the third target DNA sequence is modified within the modified-TGR so as to prevent further modification by the CRISPR/Cas9 system.

6. The method of claim 5, wherein one or more of the third PAM sequence and the third target DNA sequence are modified by one or more nucleotide change so that binding by the third gRNA and/or the Cas9n protein is prevented; or, wherein one or more of the second PAM sequence and the second target DNA sequence are modified by one or more nucleotide change so that binding by the second gRNA and/or the Cas9n protein is prevented

7. The method of any one of claims 1 to 6, wherein the disease-causing mutation is a repeat expansion.

8. The method of claim 7, wherein the repeat expansion is a trinucleotide expansion or a hexanucleotide expansion.

9. The method of claim 7 or 8, wherein the repeat expansion is at least about 30 bp long.

10. The method of any one of claims 7 to 9, wherein the repeat expansion comprises 5 or more hexanucleotide repeats, 10 or more trinucleotide repeats, more than 3 hexanucleotide repeats, more than 4 hexanucleotide repeats, or more than 5 hexanucleotide repeats.

11. The method of any one of claims 1 to 10, wherein the disease-causing modification is an amyotrophic lateral sclerosis (ALS)-causing mutation.

12. The method of any one of claims 2 to 11, wherein the disease is a repeat expansion disorder.

13. The method of claim 12, wherein the repeat expansion disorder is Fragile X Syndrome, Huntington's disease, spinocerebellar ataxia, myotonic dystrophy, myoclonic epilepsy, Friedreich's ataxia, amyotrophic lateral sclerosis (ALS), or frontotemporal dementia.

14. The method of any one of claims 1 to 13, wherein the first gRNA and the second gRNA are the same.

15. The method of any one of claims 1 to 14, wherein the first gRNA and the third gRNA or the second gRNA and the third gRNA are the same.

16. The method of any one of claims 1 to 15, wherein the first PAM sequence and the third PAM sequence, or the second PAM sequence and the third PAM sequence are the same.

17. The method of any one of claims 1 to 16, wherein the one or more of the first gRNA, the second gRNA, and the third gRNA is encoded by an episome.

18. The method of claim 17, wherein the mammalian cell is contacted with the episome first, prior to contacting the mammalian cell with the Cas9n protein.

19. The method of any one of claims 1 to 18, wherein the Cas9n protein is provided directly as an isolated protein.

20. The method of any one of claims 1 to 19, wherein the Cas9n protein is provided in the form of a nucleic acid encoding the Cas9n protein.

21. The method of claim 20, wherein the nucleic acid encoding the Cas9n protein is an RNA.

22. The method of claim 20, wherein the nucleic acid encoding the Cas9n protein is a DNA plasmid.

23. The method of claim 22, wherein the DNA plasmid is an expression vector.

24. The method of any one of claims 1 to 23, wherein the CRISPR/Cas9 system is introduced into the mammalian cell multiple times.

25. The method of claim 24, wherein the CRISPR/Cas9 system is introduced into the mammalian cell two or more times, three or more times, five or more times, ten or more times, or more than ten times.

26. The method of any one of claims 1 to 25, wherein in step b), the CRISPR/Cas9 system is introduced into the mammalian cell via transfection.

27. The method of claim 26, wherein said transfection comprises:
a) first transfecting one or more episomal vector encoding one or more of the first gRNA, the second gRNA, and the third gRNA into the mammalian cell; and
b) then transfecting the Cas9n protein or a nucleic acid encoding the Cas9n protein into the mammalian cell.

28. The method of claim 27, wherein the nucleic acid encoding the Cas9n protein is an RNA or a DNA plasmid.

29. The method of any one of claims 1 to 28, wherein one or more of the first PAM sequence, the second PAM sequence, and the third PAM sequence is partially or fully located in an intron.

30. The method of any one of claims 1 to 29, wherein the first PAM sequence, the second PAM sequence, and the third PAM sequence are independently selected from NGG, NNGRRT, NNGRRN, NNNNGATT, NNAGAAW, NAAAAC, NGG, NAG, NGCG, NGAG, NGAN, NGNG, and NTT, where R is A or G, W is A or T, and N is A, C, G, or T.

31. The method of any one of claims 1 to 30, wherein the first and/or the second gRNA are selected such that one or more of the first PAM sequence, the second PAM sequence, the first target DNA sequence and the second target DNA sequence are modified within the modified-TGR so as to prevent further modification of the modified-TGR by the CRISPR/Cas9 system.

32. The method of any one of claims 1 to 31, wherein the first, the second and/or the third gRNA are selected such that one or more of the first PAM sequence, the second PAM sequence, the third PAM sequence, the first target DNA sequence, the second target DNA sequence, and the third target DNA sequence are modified within the modified-TGR so as to prevent further modification of the modified-TGR by the CRISPR/Cas9 system.

33. The method of any one of claims 1 to 32, wherein at least one component of the CRISPR/Cas9 system is introduced into the mammalian cell by transfection of an episomal plasmid encoding the at least one component.

34. The method of claim 34, wherein the episomal plasmid further encodes a truncated protein or a tag-epitope expressed at the cell surface of transfected cells.

35. The method of claim 33 or 34, wherein the transfected cells are selected and/or purified using the truncated protein or the tag-epitope, providing a population of cells enriched for the transfected cells and/or genomic-modified cells.

36. A method for targeted genomic modification within a target genome region (TGR) in a mammalian cell, the method comprising:
a) providing a CRISPR/Cas9 system comprising:
i) a first guide RNA (gRNA) comprising a first CRISPR RNA (crRNA) and a first trans-activating crRNA (tracrRNA) linked together, the first gRNA being capable of binding with sequence specificity to a first target DNA sequence on one strand of the DNA double helix in the TGR, the first target DNA sequence to which the first gRNA binds being adjacent to a first PAM sequence;
ii) a second gRNA comprising a second CRISPR RNA (crRNA) and a second trans-activating crRNA (tracrRNA) linked together, the second gRNA being capable of binding with sequence specificity to a second target DNA sequence, the second target DNA sequence to which the second gRNA binds being adjacent to a second PAM sequence, wherein the second target DNA sequence is on the same strand of the DNA double helix as the first target DNA sequence;
iii) a third gRNA comprising a third CRISPR RNA (crRNA) and a third trans-activating crRNA (tracrRNA) linked together, the third gRNA being capable of binding with sequence specificity to a third DNA sequence on one strand of the DNA double helix in the TGR, the third target DNA sequence to which the third gRNA binds being adjacent to a third PAM sequence, wherein the third target DNA sequence is on the opposite strand of the DNA double helix from the first and the second target DNA sequences;
iv) a fourth gRNA comprising a fourth CRISPR RNA (crRNA) and a fourth trans-activating crRNA (tracrRNA) linked together, the fourth gRNA being capable of binding with sequence specificity to a fourth DNA sequence on one strand of the DNA double helix in the TGR, the fourth target DNA sequence to which the fourth gRNA binds being adjacent to a fourth PAM sequence, wherein the fourth target DNA sequence is on the opposite strand of the DNA double helix from the first and the second target DNA sequences;
wherein at least one of the first gRNA, the second gRNA, the third gRNA, and the fourth gRNA is selected such that the CRISPR/Cas9 system can only bind and/or modify the respective target DNA sequence if the respective target DNA sequence comprises a disease-causing modification or a sequence for which modification is desired; and
iv) a Cas9n protein;
and
b) contacting the mammalian cell with the CRISPR/Cas9 system such that the TGR is modified, forming a modified-TGR.

37. The method of claim 36, wherein the CRISPR/Cas9 system can only bind and/or modify the respective target DNA sequence in the mammalian cell of a patient suffering from a disease.

38. The method of claim 36 or 37, wherein:
the fourth gRNA is selected such that the CRISPR/Cas9 system can only bind and/or modify the fourth target DNA sequence if the fourth target DNA sequence comprises a disease-causing modification or a sequence for which modification is desired; and
the second and the fourth target DNA sequence are located on opposite strands of the DNA double helix and are separated by a number of nucleotides sufficient to induce double stranded break (DSB) repair.

39. The method of claim 38, wherein the second and the fourth target DNA sequence are separated by about 100 nucleotides or less than 100 nucleotides from each other.

40. The method of claim 38 or 39, wherein the second and the fourth target DNA sequence are separated by about 10 nucleotides or less, about 20 nucleotides or less, or about 50 nucleotides or less from each other.

41. The method of any one of claims 38 to 40, wherein the DSB repair introduces an indel mutation in the target genome region.

42. The method of claim 41, wherein the indel mutation knocks out or silences the disease-causing modification in the target genome region.

43. The method of any one of claims 36 to 42, wherein the third gRNA is selected such that the CRISPR/Cas9 system can only bind and/or modify the third target DNA sequence if the third target DNA sequence comprises a disease-causing modification or a sequence for which modification is desired; and
the first and the third target DNA sequence are located on opposite strands of the DNA double helix and are separated by a number of nucleotides sufficient to induce double stranded break (DSB) repair.

44. The method of any one of claims 36 to 42, wherein the third gRNA and the first gRNA are selected such that the CRISPR/Cas9 system can bind and/or modify their respective target DNA sequences even if the respective target DNA sequences do not comprise a disease-causing modification; and
the first and the third target DNA sequence are located on opposite strands of the DNA double helix and are separated by a number of nucleotides sufficient to not induce double stranded break (DSB) repair.

45. The method of claim 44, wherein the first and the third target DNA sequence are separated by more than about 100 nucleotides from each other.

46. The method of any one of claims 36 to 45, wherein the disease-causing mutation is a heterozygous mutation.

47. The method of any one of claims 36 to 46, wherein the disease-causing mutation is a point mutation.

48. The method of any one of claims 36 to 47, wherein the disease-causing mutation is a gain of function mutation.

49. The method of any one of claims 36 to 48, wherein the disease-causing mutation is a mutated SOD1 allele.

50. The method of any one of claims 36 to 49, wherein the disease is ALS.

51. The method of any one of claims 36 to 50, wherein one or more of the first gRNA, the second gRNA, the third gRNA, and the fourth gRNA is encoded by an episome.

52. The method of claim 51, wherein the mammalian cell is contacted with the episome first, prior to contacting the mammalian cell with the Cas9n protein.

53. The method of any one of claims 36 to 52, wherein the Cas9n protein is provided directly as an isolated protein.

54. The method of any one of claims 36 to 52, wherein the Cas9n protein is provided in the form of a nucleic acid encoding the Cas9n protein.

55. The method of claim 54, wherein the nucleic acid encoding the Cas9n protein is an RNA.

56. The method of claim 54, wherein the nucleic acid encoding the Cas9n protein is a DNA plasmid.

57. The method of claim 56, wherein the DNA plasmid is an expression vector.

58. The method of any one of claims 36 to 57, wherein the CRISPR/Cas9 system is introduced into the mammalian cell multiple times.

59. The method of claim 58, wherein the CRISPR/Cas9 system is introduced into the mammalian cell two or more times, three or more times, five or more times, ten or more times, or more than ten times.

60. The method of any one of claims 36 to 59, wherein in step b), the CRISPR/Cas9 system is introduced into the mammalian cell via transfection.

61. The method of claim 60, wherein said transfection comprises:
a) first transfecting one or more episomal vector encoding one or more of the first gRNA, the second gRNA, the third gRNA, and the fourth gRNA into the mammalian cell; and
b) then transfecting the Cas9n protein or a nucleic acid encoding the Cas9n protein into the mammalian cell.

62. The method of claim 61, wherein the nucleic acid encoding the Cas9n protein is an RNA or a DNA plasmid.

63. The method of any one of claims 36 to 62, wherein one or more of the first PAM sequence, the second PAM sequence, the third PAM sequence and the fourth PAM sequence is partially or fully located in an intron.

64. The method of any one of claims 36 to 63, wherein the first PAM sequence, the second PAM sequence, the third PAM sequence and the fourth PAM sequence are independently selected from NGG, NNGRRT, NNGRRN, NNNNGATT, NNAGAAW, NAAAAC, NGG, NAG, NGCG, NGAG, NGAN, NGNG, and NTT, where R is A or G, W is A or T, and N is A, C, G, or T.

65. The method of any one of claims 36 to 64, wherein the first and/or the second gRNA are selected such that one or more of the first PAM sequence, the second PAM sequence, the first target DNA sequence and the second target DNA sequence are modified within the modified-TGR so as to prevent further modification of the modified-TGR by the CRISPR/Cas9 system.

66. The method of any one of claims 36 to 65, wherein at least one component of the CRISPR/Cas9 system is introduced into the mammalian cell by transfection of an episomal plasmid encoding the at least one component.

67. The method of claim 66, wherein the episomal plasmid further encodes a truncated protein or a tag-epitope expressed at the cell surface of transfected cells.

68. The method of claim 66 or 67, wherein the transfected cells are selected and/or purified using the truncated protein or the tag-epitope, providing a population of cells enriched for the transfected cells and/or genomic-modified cells.

69. A method for targeted genomic modification within a target genome region (TGR) in a mammalian cell, the method comprising:
a) providing a CRISPR/Cas9 system comprising:
i) a first guide RNA (gRNA) comprising a first CRISPR RNA (crRNA) and a first trans-activating crRNA (tracrRNA) linked together, the first gRNA being capable of binding with sequence specificity to a first target DNA sequence on one strand of the DNA double helix in the TGR, the first target DNA sequence to which the first gRNA binds being adjacent to a first PAM sequence;
ii) a second gRNA comprising a second CRISPR RNA (crRNA) and a second trans-activating crRNA (tracrRNA) linked together, the second gRNA being capable of binding with sequence specificity to a second target DNA sequence on the other strand of the DNA double helix in the TGR, the second target DNA sequence to which the second gRNA binds being adjacent to a second PAM sequence, wherein the first and the second target DNA sequence are on opposite strands of the DNA double helix and located sufficiently close together to induce double stranded break (DSB) repair; and
iii) a Cas9n protein; and
b) contacting the mammalian cell with the CRISPR/Cas9 system such that the TGR is modified, forming a modified-TGR;
wherein the first and/or the second gRNA are selected such that one or more of the first PAM sequence, the second PAM sequence, the first target DNA sequence and the second target DNA sequence are modified within the modified-TGR so as to prevent further modification of the modified-TGR by the CRISPR/Cas9 system.

70. The method of claim 69, wherein the first and the second target DNA sequence are located within 100 nucleotides of each other.

71. The method of claim 69 or 70, wherein the first and the second target DNA sequence are separated by about 100 nucleotides or less, about 10 nucleotides or less, about 20 nucleotides or less, or about 50 nucleotides or less, from each other.

72. The method of any one of claims 69 to 71, wherein one or more of the first gRNA, the second gRNA, and the Cas9n protein can't bind to at least one strand of the DNA double helix in the modified-TGR.

73. The method of any one of claim 69 or 72, wherein one or more of the first gRNA, the second gRNA, and the Cas9n protein can't bind to either strand of the DNA double helix in the modified-TGR.

74. The method of any one of claims 69 to 73, wherein in the modified-TGR, the first PAM sequence is modified.

75. The method of any one of claims 69 to 73, wherein in the modified-TGR, the second PAM sequence is modified.

76. The method of any one of claims 69 to 73, wherein in the modified-TGR, the first target DNA sequence is modified.

77. The method of any one of claims 69 to 73, wherein in the modified-TGR, the second target DNA sequence is modified.

78. The method of any one of claims 69 to 73, wherein in the modified-TGR, the first PAM sequence and the first target DNA sequence are both modified.

79. The method of any one of claims 69 to 73, wherein in the modified-TGR, the second PAM sequence and the second target DNA sequence are both modified.

80. The method of any one of claims 69 to 73, wherein the first and the second PAM sequence are both modified.

81. The method of any one of claims 69 to 73, wherein only one of the first PAM sequence, the second PAM sequence, the first target DNA sequence and the second target DNA sequence are modified.

82. The method of any one of claims 69 to 81, wherein the modification to one or more of the first PAM sequence, the second PAM sequence, the first target DNA sequence and the second target DNA sequence comprises one or more nucleotide change in the sequence thereof.

83. The method of any one of claims 69 to 82, wherein the modification to one or more of the first PAM sequence, the second PAM sequence, the first target DNA sequence and the second target DNA sequence prevents binding of the Cas9n protein.

84. The method of any one of claims 69 to 83, wherein the modification to one or more of the first PAM sequence, the second PAM sequence, the first target DNA sequence and the second target DNA sequence prevents binding of the first gRNA.

85. The method of any one of claims 69 to 84, wherein the modification to one or more of the first PAM sequence, the second PAM sequence, the first target DNA sequence and the second target DNA sequence prevents binding of the second gRNA.

86. The method of any one of claims 82 to 85, wherein the one or more nucleotide change in the first and/or the second PAM sequence is a silent mutation that does not change the amino acid sequence encoded by the first and/or the second target DNA sequence respectively.

87. The method of any one of claims 69 to 86, wherein the CRISPR/Cas9 system further comprises:
iv) a third gRNA comprising a third CRISPR RNA (crRNA) and a third trans-activating crRNA (tracrRNA) linked together, the third gRNA being capable of binding with sequence specificity to a third DNA sequence on one strand of the DNA double helix in the TGR, the third target DNA sequence to which the third gRNA binds being adjacent to a third PAM sequence,
wherein the third target DNA sequence is located either within 100 nucleotides of the first target DNA sequence on the opposite strand of the DNA double helix or within 100 nucleotides of the second target DNA sequence on the opposite strand of the DNA double helix;
and wherein the third gRNA is selected such that the CRISPR/Cas9 system can only bind and/or modify the third target DNA sequence if the target genome region comprises a disease-causing modification or a sequence for which modification is desired.

88. The method of claim 87, wherein the CRISPR/Cas9 system can only bind and/or modify the third target DNA sequence in the mammalian cell of a patient suffering from a disease.

89. The method of claim 87 or 88, wherein the third target DNA sequence is only adjacent to the third PAM sequence if the target genome region comprises a disease-causing modification or a sequence for which modification is desired or is in the mammalian cell of a patient suffering from a disease.

90. The method of any one of claims 87 to 89, wherein the third target DNA sequence is modified within the modified-TGR so as to prevent further modification by the CRISPR/Cas9 system.

91. The method of claim 90, wherein one or more of the third PAM sequence and the third target DNA sequence are modified by one or more nucleotide change so that binding by the third gRNA and/or the Cas9n protein is prevented.

92. The method of any one of claims 87 to 91, wherein the disease-causing mutation is an amyotrophic lateral sclerosis (ALS)-causing mutation.

93. The method of any one of claims 88 to 92, wherein the disease is ALS.

94. The method of any one of claims 69 to 86, wherein the first gRNA and the second gRNA are the same.

95. The method of any one of claims 69 to 86 and 94, wherein the first PAM sequence and the second PAM sequence are the same.

96. The method of any one of claims 87 to 95, wherein the first gRNA and the third gRNA or the second gRNA and the third gRNA are the same.

97. The method of any one of claims 87 to 96, wherein the first PAM sequence and the third PAM sequence, or the second PAM sequence and the third PAM sequence are the same.

98. The method of any one of claims 69 to 97, wherein the CRISPR/Cas9 system further comprises:
v) a repair template for homology-directed repair (HDR).

99. The method of claim 98, wherein the repair template comprises one or more nucleotide change in one or more of the first PAM sequence, the second PAM sequence, and the third PAM sequence.

100. The method of claim 98 or 99, wherein the repair template comprises one or more nucleotide change in one or more of the first target DNA sequence, the second target DNA sequence, and the third target DNA sequence.

101. The method of any one of claims 98 to 100, wherein the repair template is a single-stranded DNA oligonucleotide (ssODN).

102. The method of any one of claims 98 to 101, wherein the repair template further comprises a DNA sequence to be inserted or modified in the target genome region.

103. The method of any one of claims 98 to 102, wherein the repair template is capped at the 5′ end, the 3′ end, or both.

104. The method of claim 103, wherein the cap comprises 4 nucleotides or a peptide linked to the repair template.

105. The method of any one of claims 98 to 104, wherein the repair template further comprises a tag at the 5′ end, the 3′ end, or both.

106. The method of claim 105, wherein the tag is a detectable moiety.

107. The method of claim 106, wherein the detectable moiety is a fluorophore, a cyanine dye, or a quantum dot.

108. The method of any one of claims 69 to 107, wherein the one or more of the first gRNA, the second gRNA, and the third gRNA is encoded by an episome.

109. The method of claim 108, wherein the mammalian cell is contacted with the episome first, prior to contacting the mammalian cell with the Cas9n protein and/or the repair template.

110. The method of any one of claims 69 to 109, wherein the Cas9n protein is provided directly as an isolated protein.

111. The method of any one of claims 69 to 110, wherein the Cas9n protein is provided in the form of a nucleic acid encoding the Cas9n protein.

112. The method of claim 111, wherein the nucleic acid encoding the Cas9n protein is an RNA.

113. The method of claim 111, wherein the nucleic acid encoding the Cas9n protein is a DNA plasmid.

114. The method of claim 113, wherein the DNA plasmid is an expression vector.

115. The method of any one of claims 69 to 114, wherein the CRISPR/Cas9 system is introduced into the mammalian cell multiple times.

116. The method of claim 115, wherein the CRISPR/Cas9 system is introduced into the mammalian cell two or more times, three or more times, five or more times, ten or more times, or more than ten times.

117. The method of any one of claims 69 to 116, wherein in step b), the CRISPR/Cas9 system is introduced into the mammalian cell via transfection.

118. The method of claim 117, wherein said transfection comprises:
a) first transfecting one or more episomal vector encoding one or more of the first gRNA, the second gRNA, and the third gRNA into the mammalian cell; and
b) then transfecting the Cas9n protein or a nucleic acid encoding the Cas9n protein and the repair template into the mammalian cell, the repair template being a ssODN.

119. The method of claim 118, wherein the nucleic acid encoding the Cas9n protein is an RNA or a DNA plasmid.

120. The method of any one of claims 99 to 119, wherein the one or more nucleotide change in the first, the second and/or third PAM sequence in the repair template prevents binding of the Cas9n.

121. The method of any one of claims 99 to 120, wherein the one or more nucleotide change in the first, the second and/or the third PAM sequence in the repair template is a silent mutation that does not change the amino acid sequence encoded by the respective target DNA sequence.

122. The method of any one of claims 69 to 112, wherein one or more of the first PAM sequence, the second PAM sequence, and the third PAM sequence is partially or fully located in an intron.

123. The method of any one of claims 69 to 122, wherein the first PAM sequence, the second PAM sequence, and the third PAM sequence are independently selected from NGG, NNGRRT, NNGRRN, NNNNGATT, NNAGAAW, NAAAAC, NGG, NAG, NGCG, NGAG, NGAN, NGNG, and NTT, where R is A or G, W is A or T, and N is A, C, G, or T.

124. A method for targeted genomic modification within a target genome region (TGR) in a mammalian cell, the method comprising:
a) providing a CRISPR/Cas9 system comprising:
i) one or more guide RNA (gRNA) comprising a CRISPR RNA (crRNA) and a trans-activating crRNA (tracrRNA) linked together, the one or more gRNA being capable of binding with sequence specificity to a first target DNA sequence and a second target DNA sequence in the TGR, the first target DNA sequence to which the one or more gRNA binds being adjacent to a first PAM sequence, and the second target DNA sequence being adjacent to a second PAM sequence, wherein the first and the second target DNA sequence are located within 100 nucleotides of each other and are on opposite strands of the DNA double helix;
ii) a Cas9n protein; and
iii) a repair template for homology-directed repair (HDR), wherein the repair template comprises one or more nucleotide change in one or more of the first PAM sequence and the second PAM sequence;
and
b) contacting the mammalian cell with the CRISPR/Cas9 system, such that the TGR is modified, forming a modified-TGR;
wherein the repair template is selected such that one or more of the first PAM sequence, the second PAM sequence, the first target DNA sequence and the second target DNA sequence are modified within the modified-TGR so as to prevent further modification of the modified-TGR by the CRISPR/Cas9 system.

125. The method of claim 124, wherein one or more of the one or more gRNA and the Cas9n protein can't bind to at least one strand of the DNA double helix in the modified-TGR.

126. The method of claim 124 or 125, wherein one or more of the one or more gRNA and the Cas9n protein can't bind to either strand of the DNA double helix in the modified-TGR.

127. The method of any one of claims 124 to 126, wherein in the modified-TGR, the first PAM sequence is modified.

128. The method of any one of claims 124 to 126, wherein in the modified-TGR, the second PAM sequence is modified.

129. The method of any one of claims 124 to 126, wherein in the modified-TGR, the first target DNA sequence is modified.

130. The method of any one of claims 124 to 126, wherein in the modified-TGR, the second target DNA sequence is modified.

131. The method of any one of claims 124 to 126, wherein in the modified-TGR, the first PAM sequence and the first target DNA sequence are both modified.

132. The method of any one of claims 124 to 126, wherein in the modified-TGR, the second PAM sequence and the second target DNA sequence are both modified.

133. The method of any one of claims 124 to 126, wherein the first and the second PAM sequence are both modified.

134. The method of claims 124 to 126, wherein only one of the first PAM sequence, the second PAM sequence, the first target DNA sequence and the second target DNA sequence are modified.

135. The method of any one of claims 124 to 134, wherein the modification to one or more of the first PAM sequence, the second PAM sequence, the first target DNA sequence and the second target DNA sequence comprises one or more nucleotide change in the sequence thereof.

136. The method of any one of claims 124 to 135, wherein the modification to one or more of the first PAM sequence, the second PAM sequence, the first target DNA sequence and the second target DNA sequence prevents binding of the Cas9n protein.

137. The method of any one of claims 124 to 136, wherein the modification to one or more of the first PAM sequence, the second PAM sequence, the first target DNA sequence and the second target DNA sequence prevents binding of one or more of the one or more gRNAs.

138. The method of any one of claims 124 to 137, wherein the modification to one or more of the first PAM sequence, the second PAM sequence, the first target DNA sequence and the second target DNA sequence prevents binding of two or more gRNAs.

139. The method of any one of claims 135 to 138, wherein the one or more nucleotide change in the first and/or the second PAM sequence is a silent mutation that does not change the amino acid sequence encoded by the first and/or the second target DNA sequence respectively.

140. The method of any one of claims 124 to 139, wherein the CRISPR/Cas9 system further comprises:
iv) a third gRNA comprising a third CRISPR RNA (crRNA) and a third trans-activating crRNA (tracrRNA) linked together, the third gRNA being capable of binding with sequence specificity to a third DNA sequence on one strand of the DNA double helix in the TGR, the third target DNA sequence to which the third gRNA binds being adjacent to a third PAM sequence,
wherein the third target DNA sequence is located either within 100 nucleotides of the first target DNA sequence on the opposite strand of the DNA double helix or within 100 nucleotides of the second target DNA sequence on the opposite strand of the DNA double helix;
and wherein the third gRNA is selected such that the CRISPR/Cas9 system can only bind and/or modify the third target DNA sequence if the target genome region comprises a disease-causing modification or a sequence for which modification is desired.

141. The method of claim 140, wherein the CRISPR/Cas9 system can only bind and/or modify the third target DNA sequence in the mammalian cell of a patient suffering from a disease.

142. The method of any one of claims 124 to 139, wherein the first target DNA sequence and the second target DNA sequence have the same nucleotide sequence.

143. The method of any one of claims 124 to 102, wherein the first PAM sequence and the second PAM sequence are the same.

144. The method of any one of claims 124 to 143, wherein one or more of the gRNAs are the same.

145. The method of any one of claims 124 to 144, wherein the repair template comprises one or more nucleotide change in one or more of the first PAM sequence and the second PAM sequence.

146. The method of any one of claims 124 to 145, wherein the repair template comprises one or more nucleotide change in one or more of the first target DNA sequence and the second target DNA sequence.

147. The method of any one of claims 124 to 146, wherein the repair template is a single-stranded DNA oligonucleotide (ssODN).

148. The method of any one of claims 124 to 147, wherein the repair template further comprises a DNA sequence to be inserted or modified in the target genome region.

149. The method of any one of claims 124 to 148, wherein the repair template is capped at the 5′ end, the 3′ end, or both.

150. The method of claim 129, wherein the cap comprises 4 nucleotides or a peptide linked to the repair template.

151. The method of any one of claims 124 to 150, wherein the repair template further comprises a tag at the 5′ end, the 3′ end, or both.

152. The method of claim 151, wherein the tag is a detectable moiety.

153. The method of claim 152, wherein the detectable moiety is a fluorophore, a cyanine dye, or a quantum dot.

154. The method of any one of claims 124 to 153, wherein one or more of the gRNAs is encoded by an episome.

155. The method of claim 154, wherein the mammalian cell is contacted with the episome first, prior to contacting the mammalian cell with the Cas9n protein and/or the repair template.

156. The method of any one of claims 124 to 155, wherein the Cas9n protein is provided directly as an isolated protein.

157. The method of any one of claims 124 to 156, wherein the Cas9n protein is provided in the form of a nucleic acid encoding the Cas9n protein.

158. The method of claim 157, wherein the nucleic acid encoding the Cas9n protein is an RNA.

159. The method of claim 157, wherein the nucleic acid encoding the Cas9n protein is a DNA plasmid.

160. The method of claim 159, wherein the DNA plasmid is an expression vector.

161. The method of any one of claims 124 to 160, wherein the CRISPR/Cas9 system is introduced into the mammalian cell multiple times.

162. The method of claim 161, wherein the CRISPR/Cas9 system is introduced into the mammalian cell two or more times, three or more times, five or more times, ten or more times, or more than ten times.

163. The method of any one of claims 124 to 162, wherein in step b), the CRISPR/Cas9 system is introduced into the mammalian cell via transfection.

164. The method of claim 163, wherein said transfection comprises:
a) first transfecting one or more episomal vector encoding one or more of the gRNAs into the mammalian cell; and
b) then transfecting the Cas9n protein or a nucleic acid encoding the Cas9n protein and the repair template into the mammalian cell, the repair template being a ssODN.

165. The method of claim 164, wherein the nucleic acid encoding the Cas9n protein is an RNA or a DNA plasmid.

166. The method of any one of claims 124 to 165, wherein one or more of the first PAM sequence and the second PAM sequence is partially or fully located in an intron.

167. The method of any one of claims 124 to 166, wherein the first PAM sequence, the second PAM sequence, and the third PAM sequence are independently selected from NGG, NNGRRT, NNGRRN, NNNNGATT, NNAGAAW, NAAAAC, NGG, NAG, NGCG, NGAG, NGAN, NGNG, and NTT, where R is A or G, W is A or T, and N is A, C, G, or T.

168. The method of any one of claims 1 to 167, wherein the mammalian cell is a human cell.

169. The method of any one of claims 1 to 168, wherein the mammalian cell is an embryonic stem cell, a pluripotent stem cell, an induced pluripotent stem cell, a multipotent stem cell, a directly reprogrammed multipotent stem cell, a precursor cell, a progenitor cell, or a somatic cell.

170. The method of any one of claims 1 to 168, wherein the mammalian cell is a neuronal cell, a neural progenitor cell, a neural precursor cell or a neural stem cell.

171. The method of claim 170, wherein the neuronal cell is a neuron, an astrocyte, or an oligodendrocyte.

172. The method of any one of claims 1 to 168, wherein the mammalian cell is a precursor, progenitor or stem cell of ectodermal, endodermal, or mesodermal lineage.

173. The method of claim 172, wherein the precursor, progenitor or stem cell is of cardiac lineage, blood lineage, muscle lineage, adipocyte (fat) lineage, epithelial lineage, endothelial lineage, epidermal lineage, pulmonary lineage, hepatic lineage, pancreatic lineage, or kidney (renal) lineage.

174. The method of any one of claims 1 to 173, wherein the mammalian cell is a tumour or cancer cell.

175. The method of any one of claims 1 to 174, wherein the TGR includes or is adjacent to an H46R mutation in the SOD1 gene.

176. The method of any one of claims 1 to 175, wherein the TGR comprises all or a portion of the DNA sequence set forth in region 31655770-31670821 of NCBI Reference Sequence NC_000021.9.

177. The method of claim 175 or 176, wherein the PAM sequence is 3′-GGA-5′ and the repair template comprises a single nucleotide change in the PAM sequence that changes the PAM sequence to 3′-TGA-5′.

178. The method of any one of claims 175 to 177, wherein the repair template is a ssODN having the sequence set forth in SEQ ID NO: 6 or 7.

179. The method of any one of claims 175 to 178, wherein one or more gRNA comprises the sequence set forth in SEQ ID NO: 4 or 5.

180. The method of any one of claims 175 to 179, wherein one or more gRNA is selected such that the target DNA sequence to which it binds is adjacent to the respective PAM sequence only in the mutated gene of an ALS patient and not in a non-mutated gene.

181. The method of any one of claims 1 to 174, wherein the TGR comprises all or a portion of the CCR5 gene.

182. The method of any one of claims 1 to 174 and 181, wherein the TGR comprises all or a portion of the DNA sequence set forth in region 46372903-46373961 of NCBI Reference Sequence NC_000003.12.

183. The method of any one of claims 181 to 182, wherein the repair template is a ssODN having the sequence set forth in SEQ ID NO: 10 or 11.

184. The method of any one of claims 181 to 183, wherein one or more gRNA comprises the sequence set forth in SEQ ID NO: 8 or 9.

185. The method of any one of claims 1 to 174, wherein the TGR comprises all or a portion of the CDKN2A or P16 gene.

186. The method of any one of claims 1 to 174 and 183, wherein the TGR comprises all or a portion of the DNA sequence set forth in region 21967752-21995301 of NCBI Reference Sequence NC-000009.

187. The method of any one of claims 1 to 174, wherein the TGR comprises all or a portion of the C9ORF72 gene.

188. The method of any one of claims 1 to 174 and 187, wherein the TGR comprises all or a portion of the DNA sequence set forth in region 27546546-27573866 of NCBI Reference Sequence NC_000009.12.

189. The method of claim 187 or 188, wherein one or more gRNA comprises the sequence set forth in SEQ ID NOs: 1, 2, or 3.

190. The method of claim any one of claims 1 to 189, wherein one or more of the gRNAs comprises or consists of the sequence set forth in SEQ ID NO: 1, 2, 3, 4, 5, 8, 9, 12-81, 84-103, 112 or 113.

191. A guide RNA comprising the sequence set forth in any one of SEQ ID NOs: 1, 2, 3, 4, 5, 8, 9, 12-81, 84-103, 112 and 113.

192. A guide RNA consisting of the sequence set forth in any one of SEQ ID NOs: 1, 2, 3, 4, 5, 8, 9, 12-81, 84-103, 112 and 113.

193. A ssODN comprising the sequence set forth in any one of SEQ ID NOs: 6, 7, 10, 11, 82, and 83.

194. A ssODN consisting of the sequence set forth in any one of SEQ ID NOs: 6, 7, 10, 11, 82, and 83.

195. The method of any one of the preceding claims, wherein the repair template comprises or consists of the sequence set forth in SEQ ID NO.: 6, 7, 10, 11, 82, or 83.

196. The ssODN of claim 194 or 194, further comprising a cap at the 5′ end, the 3′ end, or both.

197. The ssODN of claim 196, wherein the cap comprises 4 nucleotides.

198. The ssODN of claim 197, wherein the cap is 5′-CGCG.

199. The ssODN of any one of the preceding claims, further comprising a detectable tag selected from a fluorophore, a protein, a dye, or a quantum dot.

200. An isolated or recombinant nucleic acid encoding the guide RNA and/or the ssODN according to any one of the preceding claims.

201. The nucleic acid of claim 200, wherein the nucleic acid is an expression vector or a cloning vector.

202. A cell comprising, transfected with, or expressing the guide RNA, ssODN, or nucleic acid according to any one of the preceding claims.

203. A kit for targeted genomic modification within a target genome region in a mammalian cell, the kit comprising the guide RNA, the ssODN, and/or the nucleic acid according to any one of the preceding claims, and/or a Cas9 protein or a nucleic acid encoding the Cas9 protein; and instructions for use thereof.

204. The kit of claim 203, wherein the Cas9 protein is Cas9n.

205. A method for treating ALS or Frontotemporal Dementia in a patient in need thereof, comprising carrying out targeted genomic modification within a target genome region (TGR) in a mammalian cell of the patient as described in any one of the preceding claims, the TGR comprising all or a portion of the DNA sequence set forth in region 27546546-27573866 of NCBI Reference Sequence NC_000009.12.

206. A method for treating ALS in a patient in need thereof, comprising carrying out targeted genomic modification within a target genome region (TGR) in a mammalian cell of the patient as described in any one of the preceding claims, the TGR comprising all or a portion of the DNA sequence set forth in region 31655770-31670821 of NCBI Reference Sequence NC_000021.9.

207. A method for treating HIV in a patient in need thereof, comprising carrying out targeted genomic modification within a target genome region (TGR) in a mammalian cell of the patient as described in any one of the preceding claims, the TGR comprising all or a portion of the DNA sequence set forth in region 46372903-46373961 of NCBI Reference Sequence NC_000003.12.

208. A method for treating cancer in a patient in need thereof, comprising carrying out targeted genomic modification within a target genome region (TGR) in a mammalian cell of the patient as described in any one of the preceding claims, the TGR comprising all or a portion of a cancer-causing gene or of the DNA sequence set forth in region 21967752-21995301 of NCBI Reference Sequence NC-000009.

209. A method for treating a mitochondrial disease in a patient in need thereof, comprising carrying out targeted genomic modification within a target mitochondrial DNA region in a mammalian cell of the patient as described in any one of the preceding claims, wherein the ssODN is conjugated with MSP or TPP and the target mitochondrial DNA region comprises the nt.A12770G mutation.

210. A method of treating cystic fibrosis in a patient in need thereof, comprising carrying out targeted genomic modification within a target genome region (TGR) in a mammalian cell of the patient as described in any one of the preceding claims, the TGR comprising the W1282X mutation.

211. A method for inactivation of a transgene in a genetically-modified organism (GMO), comprising carrying out targeted genomic modification within a target genome region (TGR) in a cell of the GMO as described in any one of the preceding claims.

212. A method of treating a disease listed in Table 5 or Table 6 in a patient in need thereof, comprising carrying out targeted genomic modification within a target genome region (TGR) in a mammalian cell of the patient as described in any one of the preceding claims.

213. A method for treating a repeat expansion disorder in a patient in need thereof, comprising carrying out targeted genomic modification within a target genome region (TGR) in a mammalian cell of the patient as described in any one of the preceding claims, the TGR comprising a repeat expansion.

214. The method of claim 213, wherein the repeat expansion disorder is Fragile X Syndrome, Huntington's disease, spinocerebellar ataxia, myotonic dystrophy, myoclonic epilepsy, Friedreich's ataxia, amyotrophic lateral sclerosis (ALS), or frontotemporal dementia.

215. The method of any one of the preceding claims, wherein the targeted genomic modification is carried out in vivo or ex vivo.
